(19) United States
(12) Patent Application Publication
(10) Pub. No.: US 2006/0020398 A1
(43) Pub. Date: Jan. 26, 2006

(54) Integration of gene expression data and non-gene data

(75) Inventors: Suzanne D. Vernon, Salida, CO (US); Amarendra S. Yavatkar, Germantown, MD (US); Elizabeth Unger, Doraville, GA (US); William C. Reeves, Atlanta, GA (US); Dan Hoang Bui, Vienna, VA (US); Stanley Lucas, Rockville, MD (US)

Correspondence Address: KLARQUIST SPARKMAN, LLP, 121 S.W. SALMON STREET, SUITE 1600, PORTLAND, OR, 97204, US

(73) Assignees: The Gov.of the USA as Repted. by the Secretary of the Dept. of Health & Human Services, Centers....., Atlanta, GA (US); SRA International, Inc., Fairfax, VA (US)

(21) Appl. No.: 11/140,596

(22) Filed: May 26, 2005

Related U.S. Application Data
(63) Continuation of application No. PCT/US03/37951, filed on Nov. 25, 2003.
(60) Provisional application No. 60/429,920, filed on Nov. 27, 2002.

(51) Int. Cl. G06F 17/30; G01N 33/48; G01N 33/50; G06F 19/00

(52) U.S. Cl. 702/20; 707/1

(57) ABSTRACT

Gene expression data and non-gene data can be integrated. Data so integrated can be analyzed in a variety of ways. For example, queries based on epidemiological data can be processed to generate results. The results can be further refined and analyzed. For example, further queries can be based on gene expression criteria to identify gene expression phenomena within the results. Grouping of data into sets is supported, and analysis tools can determine feature differences between sets or otherwise present the sets in a variety of ways, including visual depiction of gene expression data.

### REFERENCE TO RELATED APPLICATIONS

This application claims the benefit of U.S. Provisional Application No. 60/429,920 to Vernon et al., entitled “INTEGRATION OF GENE EXPRESSION DATA AND NON-GENE DATA,” filed Nov. 27, 2002, which is hereby incorporated herein by reference.

### FIELD

The disclosed technologies relate to bioinformatics, such as gene expression informatics.

### BACKGROUND

Over the last decade, advances in microarray technologies have made gene expression studies increasingly reliable and accessible. These developments have dramatically enhanced the potential for complex gene expression analysis. It is now possible to simultaneously interrogate and analyze the expression of tens of thousands of genes in a single experiment. With the introduction of sophisticated laboratory instrumentation, robotics, and large, complex data sets, biomedical research is increasingly becoming a cross-disciplinary endeavor involving biologists, engineers, software designers, physicists, and mathematicians.

As the tools for imaging, quantifying, and analyzing gene expression data proliferate, researchers are provided with new opportunities for investigating relationships between and among genes. However, even though there are numerous new technologies available, researchers still have a need for additional technologies for investigating phenomena related to gene expression data.

### SUMMARY

One of the areas in which there still remains a need for additional technologies is in the area of integrating gene expression data with non-gene data.

Technologies disclosed herein can integrate gene expression data with a variety of non-gene data. Such integration can be useful for a number of applications, such as exploring relationships between gene expression data and non-gene data or exploring relationships between genes selected based on non-gene data.

As described herein, gene expression data and non-gene data (e.g., epidemiological, demographic, or both) can be integrated. Such integration can facilitate a number of analyses via a variety of tools.

Various of the tools described herein relate to query functionality. For example, gene expression data (e.g., microarray experiment results) for subjects meeting specified non-gene criteria can be requested via a query. The query results can then be further analyzed to investigate possible gene expression and non-gene relationships.

For example, the query results can be processed by further queries to determine which genes are expressed for subjects in the query results.

If desired, query results can be grouped into two or more groups. Further analysis can be performed on the groups (e.g., to determine which genes are expressed in one group but not another).

Further, a variety of visualization tools can be provided so that a researcher can better understand results from any of the queries or other analyses. For example, scatter plot and M v. A plots of gene expression information can be shown for microarray experiments associated with subjects meeting specified criteria. Various clustering algorithms (e.g., hierarchical, Kmeans, and SOM clustering) can also be supported in visualization tools.

The technologies described herein can be implemented in a client-server arrangement (e.g., for access via a network such as the Internet). Various user interface features can provide useful functionality to assist a researcher.

The technologies described herein can be useful for assisting in performing any number of analyses. Such analyses can, for example, assist in providing diagnostic and prognostic information, and profiling disease susceptibility, contagion, and the like.

Additional features and advantages of the disclosed technologies will be made apparent from the following detailed description of illustrated embodiments, which proceeds with reference to the accompanying drawings.

### BRIEF DESCRIPTION OF THE DRAWINGS

FIG. 1 shows an exemplary arrangement in which gene expression data and non-gene data are integrated.

FIG. 2 is a flowchart showing an exemplary method for performing a function on integrated gene expression and non-gene data.

FIG. 3 is a flowchart showing an exemplary method for performing analyses on integrated gene expression and non-gene data.

FIG. 4 is a block diagram showing an exemplary computer system on which technologies described herein can be implemented.

FIG. 5 is a flowchart showing an exemplary method for collecting and analyzing integrated gene expression and non-gene data.

FIG. 6 is a screen shot of an exemplary user interface by which an operation can be performed on integrated gene expression and non-gene data.

FIG. 7 is a screen shot of an exemplary user interface by which results of an operation (e.g., such as that of FIG. 6) on integrated gene expression and non-gene data are presented.

FIG. 8 is a block diagram of an analysis session performed via the described technologies.

FIG. 9 is a flow chart of an exemplary method for obtaining gene expression data.

FIG. 10 is a screen shot showing an exemplary user interface for specifying a query.

FIG. 11 is a screen shot showing an exemplary user interface for providing results of a query.

FIG. 12 is a screen shot showing an exemplary user interface for performing microarray expression of the results of a query.

FIG. 13 is a screen shot showing an exemplary user interface for presenting the results of a microarray expression query.

FIG. 14 is a screen shot showing an exemplary user interface for presenting a scatter plot showing gene expression information for two or more microarrays.

FIG. 15 is a screen shot showing an exemplary user interface for presenting an M v. A plot.

FIGS. 16, 17, 18, 19, 20, and 21 are together a block diagram showing an exemplary relational database schema of an exemplary implementation of the technologies.

FIG. 22 is a screen shot during exemplary operation of an exemplary implementation of technologies described herein. Some of the text in FIG. 22 is also shown in FIG. 42

FIG. 23 is a screen shot of during exemplary operation of an exemplary implementation of technologies described herein whereby non-gene criteria can be specified via a user interface. Some of the text in FIG. 23 is also shown in FIG. 50.

FIG. 24A is a screen shot during exemplary operation of an exemplary implementation of technologies described herein showing results of a query (e.g., with the criteria entered via a user interface shown in FIG. 23).

FIG. 24B is a screen shot of an exemplary microarray image.

FIG. 24C is a screen shot of an exemplary histogram associated with a microarray image.

FIG. 25A is a screen shot showing an exemplary summary of data for selected microarray experiments.

FIG. 25B is a screen shot showing data such as that of FIG. 25A in an exemplary spreadsheet format.

FIG. 25C is a screen shot showing an exemplary summary of expression information.

FIGS. 26A and 26B are a screen shots showing a Microarray Expression Query Tool Form during exemplary operation of an exemplary implementation of technologies described herein. Some of the text in FIG. 26A is also shown in FIGS. 58A-D. Some of the text in FIG. 26B is also shown in FIG. 53D.

FIGS. 27A-D, 28, 29, 30, 31, 32, 33, 34, and 35 are screen shots showing various features during exemplary operation of an exemplary implementation of technologies described herein. Some of the text in FIGS. 27A, 27B, and 33 is also shown in FIG. 54A. Some of the text in FIG. 28 is also shown in FIGS. 46 and 65. Some of text in FIG. 32 is also shown in FIGS. 53A-D. Some of the text in FIG. 34 is also shown in FIG. 52. Some of the text in FIG. 35 is also shown in FIG. 51.

FIGS. 36-73 are screen shots depicted in the single intensity and dual probe user manuals depicting various features during exemplary operation of an exemplary implementation of technologies described herein.

### DETAILED DESCRIPTION

### EXAMPLE 1

### Exemplary Overview

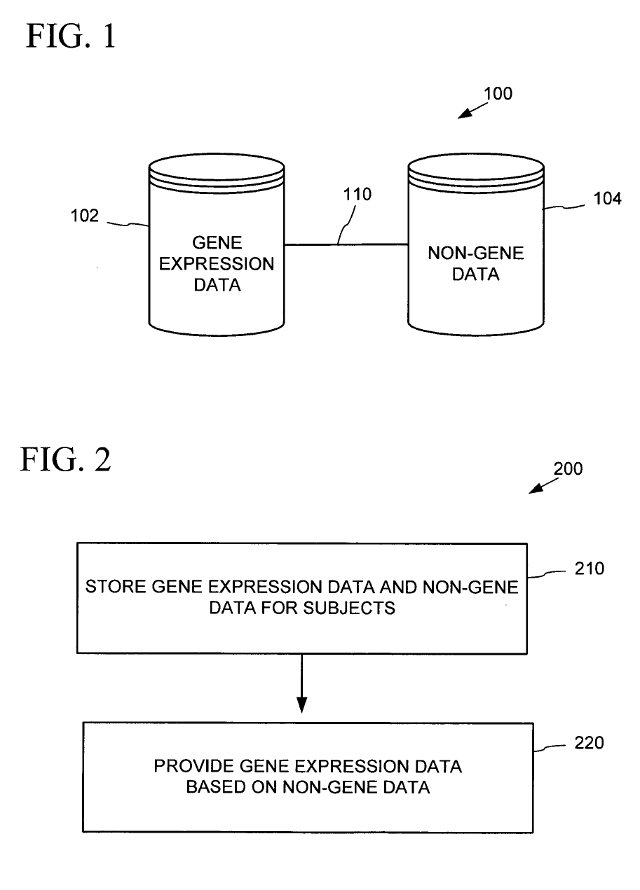

FIG. 1 shows an exemplary overview of an arrangement 100 in which gene expression data 102 is integrated with non-gene data 104. Although two databases are shown, such an arrangement can be implemented with one or more databases (e.g., the data can be integrated in a single database or the gene expression data 102 can be in one or more databases and the non-gene data 104 can be in one or more databases). Any of the databases can take the form of one or more tables or other arrangements (e.g., XML or the like).

The linking mechanism 110 serves to integrate the two disparate forms of data. The linking mechanism can take many forms, such as one or more linking fields or one or more linking tables. As described below, a variety of functions can be performed on the integrated data, any of which can take advantage of the linking mechanism 110.

### EXAMPLE 2

### Exemplary Gene Expression Data

In any of the examples described herein, gene expression data can include any information indicating the presence, absence, or level of a particular nucleic acid. Gene expression data may be provided by any experiment in which hybridizations can be detected or measured (e.g., a microarray experiment measuring single intensity or dual probe hybridizations, or from immobilized targets). Various detection methods (e.g, radioactive, chemiluminescent, or fluorescent methods) can be used.

Commercial microarrays may be obtained for nucleic acids representing any set of genes of interest. In a microarray, a spot that has hybridized to a nucleic acid provided to the array from a biological sample from a subject can be called a “feature.” A feature on the microarray is a signal representing a nucleic acid that the patient sample is expressing. The signal thus both identifies and provides a definition of the nucleic acid expressed in the biological sample of the subject. Thus, a feature in a microarray represents a nucleic acid expressed by a subject.

Gene expression data can comprise a gene expression table having gene expression data for various microarray experiments, which can be linked to particular subjects via a linking field, linking table, or some combination thereof. If desired, the gene expression data can be grouped by study or other characteristic.

In the case of single intensity data, any single intensity data can be used (e.g., data generated from a gold label), including genomic, proteomic, metabolomic, or other -omic data. A variety of detection techniques (e.g., relative light scattering) can be used to acquire such single intensity data.

### EXAMPLE 3

### Exemplary Non-Gene Data

In any of the examples described herein, non-gene information can include any data related to a biological subject (e.g., a human subject), such as epidemiological data for the subject, demographic data for the subject, or some combination thereof.

Epidemiological data can comprise, for example, disease or condition-related information, body mass index (“BMI”), clinical indicia, clinical test results, disease or condition study (e.g., whether the subject is a control subject or disease subject), date of sample, disease symptoms (e.g., presented symptoms such as sore throat, muscle weakness, and the like), disease status information (onset, stage, duration, and the like), therapeutic treatment information, drug regimens, or some combination thereof). Demographic data can comprise, for example, gender, age, race, geographic location, geographic residency, occupation, military service details, income level, social class, and the like.

Other non-gene data can include study identification, case/control classification, and correlates, such as a disease state or whether the subject has been exposed to or infected with a infectious agent (e.g., virus) known or believed to be correlated with a condition.

Non-gene information may also be other forms of disparate information that is not in the same form as gene expression data, including textual information databases, chemical structure data databases, databases containing graphics or patterns, or other forms of information contained in a database that are disparate to gene expression data. If desired, the non-gene data can take the form of any data elements common for a particular disease, state, or organism.

The non-gene data can be stored in database tables (e.g., having epidemiological characteristics, demographic characteristics, or some combination thereof for subjects). The non-gene data can be linked to the gene expression data via a linking field, a linking table, or some combination thereof (e.g., by linking the microarray experiment results to a particular subject for whom non-gene data is stored). Queries comprising one or more non-gene criteria (e.g., criteria specified for any combination of non-gene characteristics or other non-gene data) can then be performed on the database tables.

### EXAMPLE 4

### Exemplary Function

One of a variety of possible functions that can be performed via the arrangement described in Example 1 is shown in a flowchart 200 for FIG. 2. The actions shown in the example can be performed by software (e.g., via a computer executing computer-executable instructions specifying the actions). At 210, gene expression data and non-gene data is stored for subjects (e.g., human participants in a study). At 220, gene expression data is provided based on non-gene data. Such an action can be implemented by performing a query (e.g., a query is performed against a combination (e.g., join) of the gene expression data and the non-gene data). For example, a query can request gene expression data for subjects having non-gene data (e.g., non-gene characteristics) meeting one or more criteria.

### EXAMPLE 5

### Exemplary Analyses

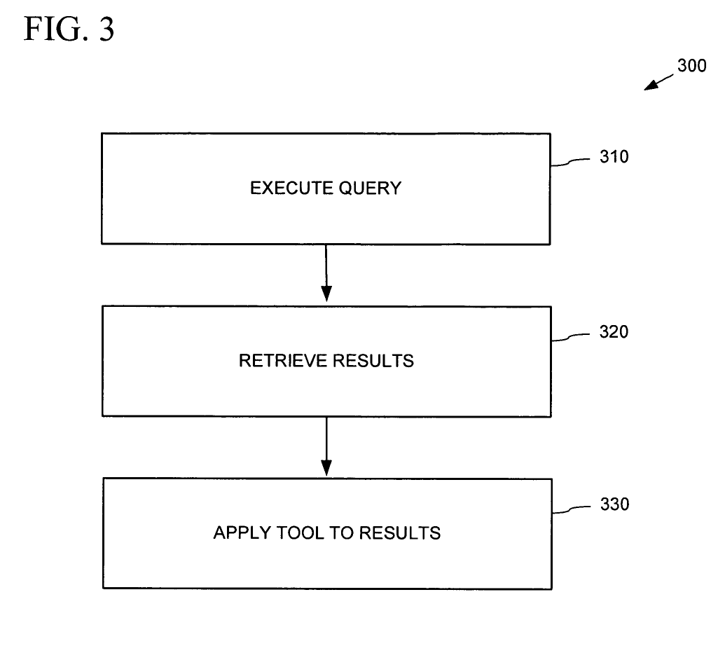

FIG. 3 shows a flowchart 300 depicting one of many exemplary analyses that can be implemented via the query functionality described in Example 4. The actions shown in the example can be performed by software (e.g., via a computer executing computer-executable instructions specifying the actions).

At 310, a query is executed. For example, the query described with reference to FIG. 3 can be executed. At 320, results appropriate for the query (e.g., the gene expression data for subjects meeting the query criteria) are retrieved (e.g., by a query engine). In practice, the results are typically a subset of the full set of gene expression data (e.g., the gene expression data for those subjects meeting the query criteria). However, a query can be formulated to provide a full set as the results, or the query step can be skipped entirely.

At 330, one or more tools can be applied to the results to facilitate analysis. Various user interfaces (e.g., graphical user interfaces) can be displayed by software to assist in specifying queries and selecting tools.

Via various analyses, a researcher can discover gene expression associated with one or more non-gene characteristics. For example, via queries, the computer can output gene expression data (e.g., microarray data) for subjects having non-gene characteristics specified in the query. Further tools can be provided to further process the gene expression data.

### EXAMPLE 6

### Exemplary Computer System(s)

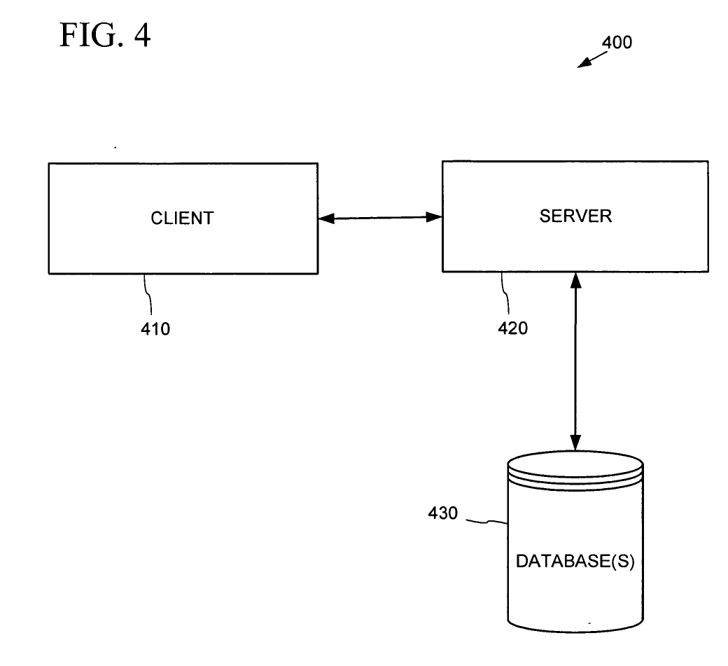

Although the described technologies can be implemented in a single computer, FIG. 4 shows an exemplary alternative arrangement 400. In the example, one or more client machines 410 access one or more server machines 420, which have access to one or more databases 430 (e.g., such as those described in Example 1). The client 410 and the server 420 can be linked via a network (e.g., a local area network or the Internet). If desired, communication over the network can be achieved by a variety of protocols (e.g., HTTP). Any of the user interfaces described herein can be presented on any of the machines, such as the client 410.

The machines 410 and 420 shown can take any of a variety of forms, including commonly-available desktop or server computer systems or other devices capable of receiving input and providing output (e.g., handheld devices). Any number of a variety of operating systems can be used, including proprietary or open-source systems.

If desired, functionality for the server 420 can be divided in a variety of ways. For example, a separate server can be provided to handle web-related (e.g., HTTP) functions, or plural servers can be used to balance the load from the clients 410.

The databases 430 can be implemented via one or more separate servers, if desired. Any databases 430 can take any of a variety of forms, including commercially-available databases including query engines implementing various optimization techniques.

### EXAMPLE 7

### Exemplary Method for Collecting and Analyzing Integrated Gene Expression and Non-Gene Data

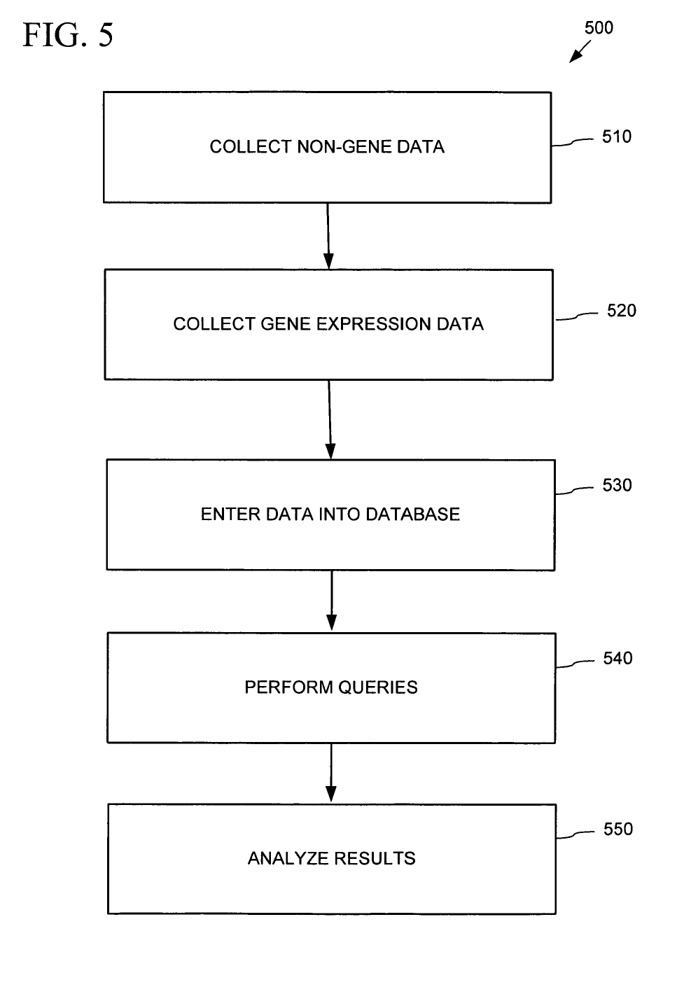

FIG. 5 shows an exemplary method 500 for collecting and analyzing integrated gene expression and non-gene data. The actions shown in the example can be performed by software (e.g., via a computer executing computer-executable instructions specifying the actions).

At 510, non-gene data is collected for a set of subjects. For example, data can be collected via subject questionnaires, subject interviews, subject medical (e.g., physical) examination, or some combination thereof.

At 520, gene expression data is collected for the set of subjects. For example, clinical samples (e.g., biological specimens such as blood) can be collected for the same population and microarray experiments performed on the samples to obtain microarray data (e.g., data indicating gene expression levels for a plurality of genes).

At 530, the data is entered into database(s). For example, microarray data can be normalized and integrated with the non-gene data. Such integration can be achieved, for example, by using a common subject identifier for both the gene and non-gene data. Or, a linking table can link an identifier (e.g., experiment number) of a microarray experiment (e.g., for a particular subject) with a subject identifier (e.g., for the same subject).

At 540, one or more queries can be performed on the data. For example, a subset of the microarray data (e.g., a subset of the experiments) can be selected by specifying various non-gene criteria (e.g., relating to the questionnaires or the physical examinations).

At 550, the results of the queries can be analyzed. For example, a tool can be applied to the results of the queries. In some cases, a visualization tool can help a researcher spot certain trends or other phenomena. As a result of spotting a trend or other phenomena, the researcher can refine or otherwise alter the query in an attempt to isolate various variables and find correlations between the non-gene data and the gene expression data. Iterative application of the tools can be supported (e.g., applying a tool to the results of another or the same tool).

### EXAMPLE 8

### Exemplary User Interface for Performing an Operation on Integrated Gene Expression and Non-Gene Data

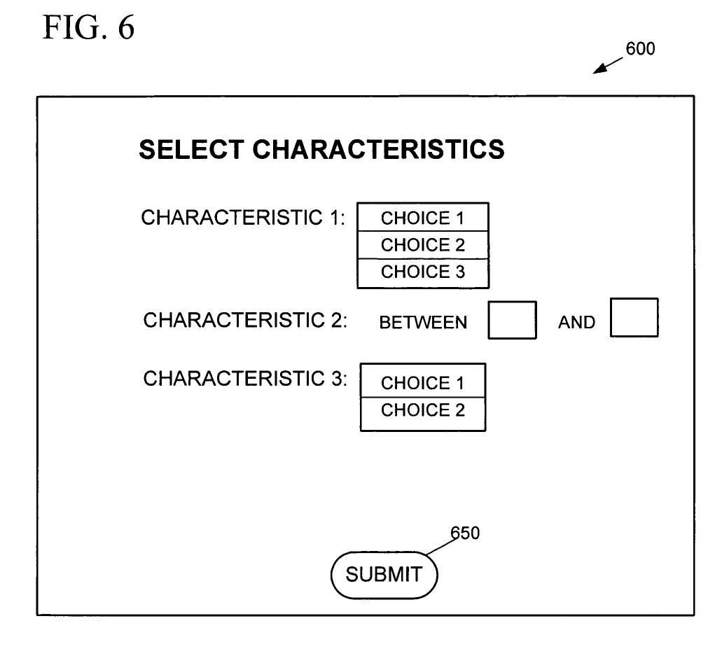

FIG. 6 shows a screen shot 600 of a user interface by which an operation can be performed on integrated gene expression and non-gene data. In the example, a query can be performed by specifying subject characteristics (e.g., non-gene characteristics). For example, various criteria (e.g., ranges, maxima, minima, and the like) can be specified for the characteristics via user interface elements (e.g., list boxes, checkboxes, edit boxes, and the like). Upon activation of the query (e.g., via the user interface element 650), results are returned.

As an alternative to the illustrated arrangement, any number of other approaches can be used to specify criteria. For example, any number of Query by Example or Structured Query Language approaches can be used.

The user interfaces described in the examples can help a researcher interact with gene expression data in a number of ways that are helpful for finding related genes, drug efficacy, and for evaluating disease management issues such as immunization, treatment, and the like.

### EXAMPLE 9

### Exemplary User Interface for Presenting Results of an Operation on Integrated Gene Expression and Non-Gene Data

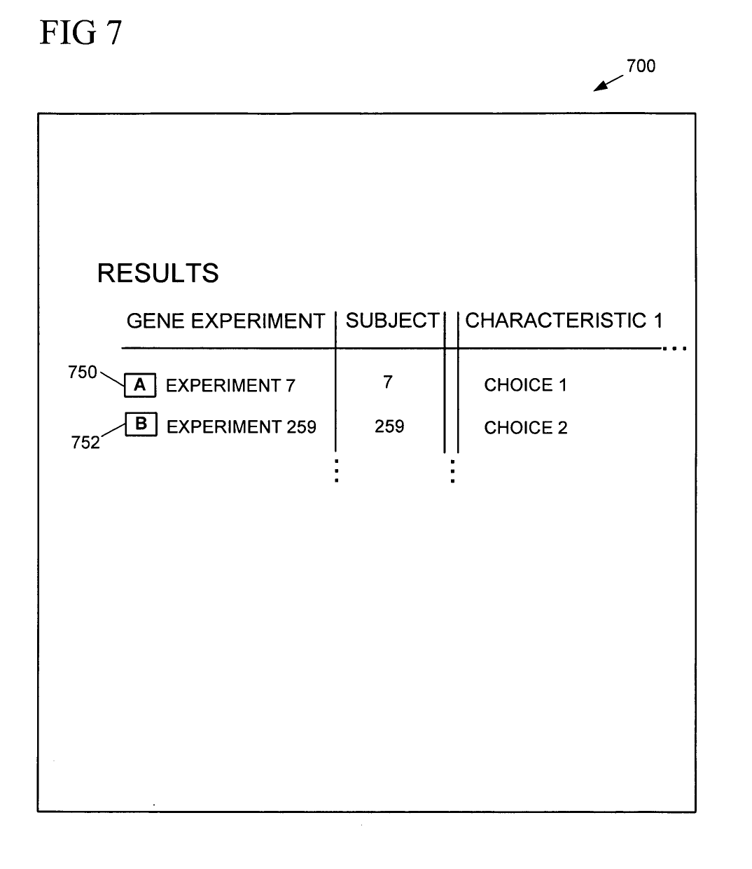

FIG. 7 shows an exemplary screen shot 700 depicting results of an operation on integrated gene expression and non-gene data. For example, the results of the query described in Examples 4 or 8 can be presented.

In the example, a representation of the gene expression data (e.g., for a particular microarray experiment) is presented in the form of an icon 750 or 752. Upon activation of the icon, further details (e.g., an image or histogram of the microarray data) are displayed. For convenience of the researcher, other gene expression data (e.g., the name of the associated microarray experiment) can be shown. Instead of the depicted results, a variety of other forms can be used (e.g., a numerical representation of expression for a particular gene).

In addition, other information can be displayed to accompany the gene expression data. For example, a subject identifier and the related subject characteristics (e.g., non-gene data).

In order to better analyze the results, a variety of tools can be provided (e.g., for visualizing, summarizing, or construction reports of the gene expression results). If desired, various groupings (e.g., between control and study individuals) can be provided. In addition, the results can be refined (e.g., a query performed on the results) to further subset the gene expression data.

Further, user interface elements (e.g., icons, hyperlinks, and the like) can be provided for searching for related information in external databases (e.g., GenBank, SwissProt, EMBL, and the like). For example, upon clicking on a gene name, a relevant entry in an external database can be displayed (e.g., in a web browser).

### EXAMPLE 10

### Exemplary Pre-Processing of Data

Techniques may be provided for pre-processing of the gene expression or non-gene data. For example, normalization techniques can be applied to gene expression data. Also, estimation of missing values can be performed.

### EXAMPLE 11

### Exemplary Tools

Various tools can be used for performing operations and analyzing the results of operations performed on integrated gene expression and non-gene data. Such tools can be provided by various user interfaces (e.g., HTTP-based user interfaces). Query functionality can be provided via tools, and the tools can include other analyses (e.g., comparison, statistical, and visual analysis tools).

Exemplary tools having query functionality include queries for microarrays from subjects having specified non-gene (e.g., epidemiological or demographic) criteria; selecting groups of microarray performed for specific subjects; clustering of genes satisfying query criteria (e.g., gene expression critera); and selection of sets of genes (e.g., based on gene name or identifier).

Other exemplary tools include group comparisons, discriminant analyses, group discovery, cluster analyses, expression distributions, quantile-quantile plots, scatter plots, visual comparisons via scatter plots, visual comparisons via M v. A plots, principal component analysis, multi-dimensional scaling, visual exploratory analysis of correlation matrix, discriminate analysis, significance tests (e.g., t-test, paired t-test, F-test), validation via permutation tests, hierarchical clustering, Kmeans clustering, and Self Organizing Maps (“SOM”) clustering.

Upon application of a tool, a user interface can provide an option to apply another (or the same) tool as selected by a user. In this way, iterative analysis can be performed by stringing together a selected set of tools.

So, for example, tools can include query functionality to query within results (e.g., adding further non-gene restrictions or gene-related restrictions). In addition, queries can be used within microarray data to determine which features are present (e.g., which genes are expressed).

Further, queries can be used within microarray data to limit the data to those features meeting a specified criteria (e.g., gene name).

Still further, the tools can be applied to groups, so that comparison between groups can be achieved (e.g., which genes are expressed in group A but not group B).

Other functionality can be provided as shown in the examples.

### EXAMPLE 12

### Exemplary Web-Based Implementation

Any of the technologies described herein can be implemented in a web-based environment. For example, the various user interfaces can be presented via web-based techniques, such as HTTP, the Common Gateway Interface (“CGI”), HTML forms, Java-related technologies (e.g., software developed via the Java Development Kit of Sun Microsystems or others), and the like. If desired, the technologies can thus be made available over a network, such as an intranet, extranet, or the Internet (e.g., the World. Wide Web), to any client machine having appropriate web browser software.

Any of the user selections described herein can be implemented via user interfaces using HTML (e.g., HTML forms). For example, user interface elements (e.g., checkboxes, edit boxes, drop down lists, and the like) can be used to collect criteria for queries in any of the examples.

If desired, security mechanisms can be provided for gathering, storing, and managing the gene expression and non-gene data. For example, the system can implement the secure socket layer (“SSL”) protocol for client-server encrypted data exchange.

### EXAMPLE 13

### Exemplary Use of Controls

A useful implementation of the described technologies includes collecting information as part of a study (e.g., a disease study). In such an implementation, gene expression and non-gene data are collected for both diseased subjects (e.g., sometimes called “case” or “study” subjects) and control subjects. The database can include data indicating whether a subject is a diseased subject or a control subject. In this way, comparative analyses of the gene expression profiles between healthy subjects and subjects with a disease can be performed (e.g., via queries, tools, and the like).

### EXAMPLE 14

### Exemplary Analysis Session

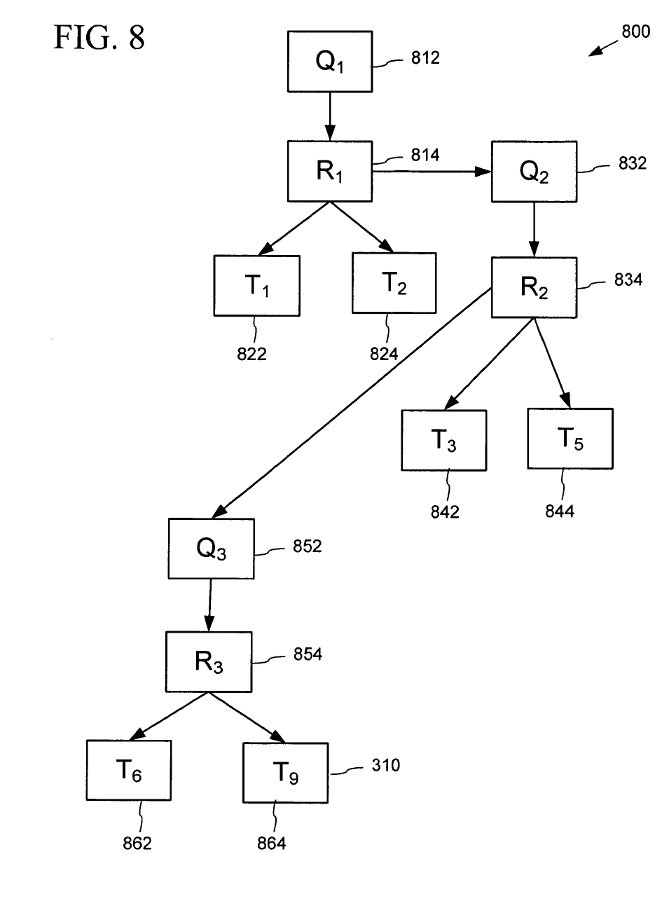

Using the technologies described herein, a researcher can conduct an analysis session to discover relationships between gene expression and non-gene data. FIG. 8 depicts an exemplary analysis session 800. At 812, the researcher performs a query on integrated gene expression and non-gene data (e.g., by specifying epidemiological or demographic criteria). At 814, the results of the query (e.g., gene expression data from subjects meeting the criteria) are provided.

Having been provided with the results, a researcher can select various tools to analyze or visualize the results (e.g., either as a group, one sub-group vis-à-vis another sub-group, or individual records within the group). For example, a tool 822 can provide information about a selected subject (e.g., the image representing a microarray experiment for the subject) and another tool 824 can provide information about the results by comparing one sub-group to another (e.g., gene expression for control subjects vis-à-vis gene expression for study subjects).

Upon consideration of the results 814, the researcher can decide to run another query similar or dissimilar to the first query 812 (e.g., based on the information gleaned from the tools). Or, as shown, the researcher can run another query on the results 814 at 832. Accordingly, the query is run against the results of the first query from 812. Upon completion of the query of 832, refined results 834 are presented. As before, tools 842 and 844 can be used to analyze or visualize the results. In this way, nested queries and analysis can be performed. Any arbitrary level of nesting can be performed.

Additionally, gene expression criteria can be specified in a query. For example, the query 852 can be executed on the refined results 834 (or the results 814) to determine which genes are expressed in the results (e.g., within the results or within groups within the results). The feature results 854 can then be further analyzed by other tools. Such tools can determine, for example, which genes are expressed in one group but not another (or expressed in both groups).

Grouping can be performed via criteria such as whether a subject is a case subject or a control subject. Other grouping by any other criteria (e.g., non-gene criteria, such as disease state) is possible.

If desired, the results (e.g., from 814 or 834) can be saved (e.g., with a name) for later retrieval. In this way, particularly informative results can be saved for sharing or additional analysis.

### EXAMPLE 15

### Exemplary Tools

For any of the tools described herein, a variety of techniques can be applied. For example, when performing a query, the results can be grouped into two or more groups (e.g., control/study and the like). A tool can compare gene expression information for the two groups in an attempt to find differences in gene expression. Such differences can be useful, for example, for designing a diagnostic.

When results are provided to a tool, one or more manual mechanisms (e.g., a list box listing microarray experiments) can be provided by which a researcher can indicate an arbitrary set of subjects. Microarray data for the subjects can then be analyzed by the tool.

For example, a query Q can be run to provide results R (e.g., gene expression data for microarray experiments related to subjects having non-gene characteristics meeting specified criteria). In a tool designed for one-to-many analysis, gene expression for a particular microarray experiment from the results R can be selected and analyzed (e.g., compared) against one or more other particular microarray experiments from the results R.

In a tool designed for many-to-many comparison, plural experiments can be analyzed against plural other experiments from the results R.

If desired, the entire gene expression data (e.g., the entire set of experiments) can be included in the results. For example, the query step can be skipped so that a tool is run on the entire set records (e.g., for a project).

Another type of tool provides a way to query within microarray results to identify which of the features (e.g., nucleic acids or genes) are present in the microarray results. In this way, a researcher can investigate relationships between genes expressed and non-gene data, such as epidemiological or demographic data.

The tools can apply a variety of statistical techniques, visualization techniques, or some combination thereof. In some implementations, color can be used to differentiate visual elements (e.g., in a scatter plot) belonging to different groups or having different ranges of values.

### EXAMPLE 16

### Exemplary Method of Collecting Gene Expression Data

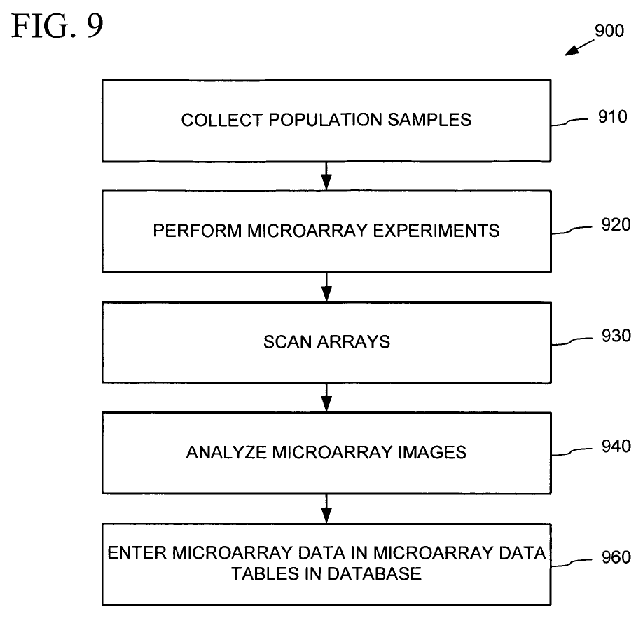

FIG. 9 is a flowchart showing an exemplary method for collecting gene expression data that can be used for any of the examples described herein. At 910, population samples (e.g., clinical specimens such as subject blood samples) are collected (e.g., at the same time as interviews and clinical examinations). At 920, microarray experiments are performed via the specimens (e.g., via hybridization). At 930, the arrays are scanned (e.g., to generate an image). At 940, the microarray images are analyzed to identify and quantify spot data.

At 960, microarray data is entered into appropriate microarray tables in a database (e.g., based on gene spot position, array, and experiment data). The database can then be queried for features representing nucleic acids that are expressed in the subject samples.

A wide variety of microarray techniques can be used, including those not yet developed. For example, single intensity and dual intensity approaches can be implemented. Further, normalization of the data can be accomplished to facilitate comparison between subjects and between studies.

### EXAMPLE 17

### Exemplary Microarray Data Acquisition Techniques

A variety of techniques can be used for acquiring microarray data. For example, study subject samples and control subject samples can be prepared by taking biological samples (e.g., blood samples) from subjects. Microarray experiments can be performed for the samples by preparing, hybridizing, and washing the microarrays. Then, images of the microarrays can be scanned to collect and process the microarray data (e.g., as shown in FIG. 9).

A variety of microarrays can be used. For example, the BD ATLAS Glass Human 3.8 I & II, 1.2 oligo arrays marketed by BD Biosciences Clontech (Becton, Dickinson and Company) of Palo Alto, Calif. Alternatives are available from a variety of sources, including MWG Biotech Inc. of High Point, N.C.; Amgen, Inc. of Thousand Oaks, Calif.; and The KTH Royal Institute of Technology of Stockholm, Sweden; and the like.

Arrays may consist of nucleic acids or cellular constituents depending on whether the arrays of interest are for determining gene expression or for identifying particular genes, respectively.

To perform the microarray experiments, RNA can be extracted from the sample and labeled (e.g., via an enzymatic method). Labeled DNA or RNA results. For example, RNA can be labeled with reverse transcription to produce labeled cDNA that is hybridized to the array. A variety of labels can be used (e.g., an affinity label such as biotin that is detected with avidin linked to gold). Based on the label used, an appropriate scanning technique can be used.

After hybridization and washing, microarray image scanning can be performed via a variety of software and hardware (e.g., a GENEPIX microarray scanner and associated software marketed by Axon Instruments, Inc. of Union City, Calif. for fluorescent labels; or a GSD-501 scanner and associated software marketed by Genicon Sciences Corporation of San Diego, Calif. for Resonance Light Scattering gold particles).

The microarray images are then analyzed by analysis software (e.g., Bionumerics software marketed by Applied Maths US of Austin, Tex.; GENEPIX software marketed by Axon Instruments, Inc. of Union City, Calif.; ARRAYVISION software marketed by Imaging Research, Inc. of St. Catharines, Ontario, Canada; or the like).

Gene spot identification and quantification can be performed before the microarray data is entered into microarray data tables. A data synchronization step can be performed in which experiment data and gene spot position is saved as character data and correlated with particular gene names and experiments.

A wide variety of commercially-available software packages for image scanning, analysis, and processing can be utilized with the technologies (e.g., BioDiscovery's ImaGene Image Analysis Software from BioDiscovery, more information at http://www.biodiscovery.com/software.html; ScanAlyze, Brown Lab's Image Analysis software, more information at http://bronzino.stanford.edu/ScanAlyze; GeneChip LIMS data warehouse, Affymetrix, more information available at http://www.affymetrix.com/products/lims/lims.html; Searchable database of published yeast microarray data, Brown Lab, Stanford University, more information at http://cmgm.stanford.edu/pbrown/explore/; Database schema and software tools for analysis of high-throughput gene expression data, MicroArray Project, NIH, more information at http://www.nhgri.nih.gov/DIR/LCG/15K/HTML/dbase.html; Resolver data warehouse & analysis software, Rosetta Inpharmatics, more information at http://www.rosetta.org/; GeneSpring data warehouse & analysis software, Silicon Genetics, more information at http://www.sigenetics.com/GeneSpring/Overview.htm).

An exemplary implementation can glean microarray data generated from the GENEPIX software analysis program of Axon, Incorporated of Union City, Calif., an independent, analysis platform for DNA and protein microarrays, tissue arrays and cell arrays. For example, upon specifying a GENPIX software file, the appropriate entries can be made into databases to reflect the microarray data (e.g., gene expression information for experiments associated with particular subjects).

Software (e.g., the Bionumerics, GenePix, ArrayVision, or similar array image analysis software mentioned above) can be used to calculate the signal intensity from the foreground and the background of the spot segmentation. Segmentation can differentiate the pixels within a spot-containing region into foreground (e.g., true signal) and background.

Software (e.g., the Affymetrix Microarray Suite “MAS” Software from Affymetrix, Inc. of Santa Clara Calif. can be used, for example, in conjunction with their GENEARRAY Scanner) to calculate relative abundance of a gene from the average difference of intensities between matching and mismatched probe-pairs designed to hybridize a particular sequence. Image files are analyzed and data generated with software (e.g., one of the programs mentioned above). The data is put into proper form for entering in the database tables (e.g., via a web enabled upload interface) along with experiment data and gene spot position. The experiment (e.g., an experiment name) can also be entered into the tables.

### EXAMPLE 18

### Exemplary Epidemiological Data in Disease Study

An exemplary implementation of the technologies involved a disease study for chronic fatigue syndrome (“CFS”). Accordingly, appropriate epidemiological data and demographic data was used as non-gene data (e.g., the non-gene data 104 of FIG. 1). Microarray data was used as the gene expression data (e.g., the gene expression data 102 of FIG. 1).

The method 500 of FIG. 5 was implemented to collect the microarray and epidemiological data in a study of a population exhibiting CFS. Control subjects (e.g., not exhibiting CFS) were also included. Researchers can search microarray data for microarrays matching selected criteria taken from the non-gene data (e.g., based on epidemiological criteria).

Information was gathered from subjects based on questionnaires designed for the study in which demographic data was obtained. Medical practitioners conducted a clinical examination of the subjects to obtain medical and clinical data at the time of interview.

The non-gene data collected included the following demographic data: gender, age, geographic location, occupation, military service, income level, social class, and race. The non-gene data also included the following epidemiological data: whether subject is a control or a disease subject, date of interview, date of clinical examination, symptoms, including sore throat, muscle weakness, fever, poor concentration, headache, malaise, tender lymph nodes, duration of symptoms, type of onset of disease, disease stage, treatment, drug regimens, other disease presentation.

Alternative arrangements are possible. For example, in another study of CFS or another disease, fewer, other, or more non-gene characteristics can be included.

### EXAMPLE 19

### Exemplary Implementation of Query Functionality

In an exemplary implementation, a researcher can query the integrated gene expression and non-gene data via various graphical interfaces. Queries can request microarray data based on epidemiological or demographic data contained in data tables in the database.

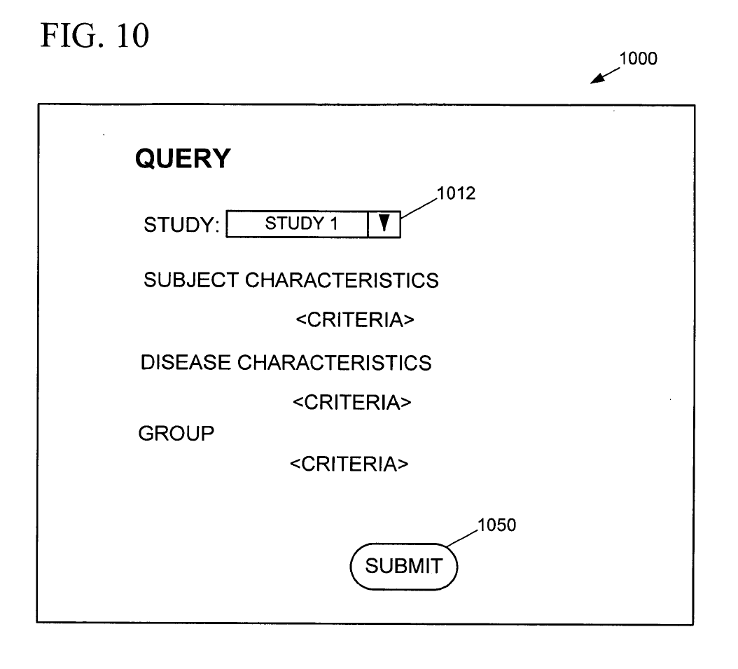

FIG. 10 shows a screen shot 1000 of an exemplary interface for specifying a query. The interface appears as a form (e.g., an HTML-based form) for which the user can supply values.

In the screen shot 1000, some of the form values related to criteria have been entered. The form has four main selection options for entering certain criteria with which to query the microarray data: Study, Subject Characteristics, Disease Characteristics, and Date of Sample. Data fields can be accessed via user interface elements such as drop-down lists, check boxes, and edit boxes. Multiple criteria for selection are permitted. The Study option allows a user to specify a project (sometimes called a “study”) via the drop down list 1012. Internally, the data can be grouped by project via a project identifier (e.g., a parent key for identifying a group of epidemiological and microarray information for subjects associated with the project). In this way, the researcher can limit the analysis to a particular project.

The Subject Characteristics options allow specification of criteria to choose subjects that meet specific demographic status criteria. Subject Characteristics criteria can include age (e.g., age boxes allow selection of a specific age or minimum and maximum ages for subjects in a group), gender, BMI (to select subjects with specific ranges of Body Mass Index), and race. Subjects can be specified as being either a disease case or a control (case/control). Or, cases and controls can be grouped separately.

Similarly, criteria related to one or more Disease Characteristics can be selected. Disease Characteristics may include, for example, typical options related to clinical presentations, disease stage, and drug history.

Date of Sample (not shown) is the date on which the subject clinical sample was obtained for microarray processing, and is specified using greater than, less than, or date range values. A series of drop-down lists allows the user to select specific dates, using the =, <, or > symbols, corresponding with the month, day, and year drop-down lists. A “Sample Dated Between” radio button allows the user to specify a date range for the query. A “Don't Check” option allows bypass of the date field (e.g., to disregard the date field during the query).

The criteria options displayed on the form can vary depending on the project selected. For example, a previous screen to the one shown can allow selection of a project. Depending on the project selected, appropriate criteria options (e.g., user interface elements for specifying criteria) are displayed. The appropriate criteria options can be stored in the database so that the technology is extensible to other projects (e.g., having other criteria, such as different, additional, or fewer non-gene criteria).

Upon activation of the query (e.g., via the submit button 1050), the microarray information associated with subjects having the specified criteria are displayed (e.g., in a user interface). In some cases, it may be desirable to identify the microarray information via a name (e.g., of the subject or the microarray experiment name) in the results. Additional tools can be optionally used to further query the retrieved arrays for reiterative examination of the retrieved gene expression profiles. For example, gene expression data for particular nucleic acids (e.g., genes) can be selected.

### EXAMPLE 20

### Exemplary Grouping of Gene Expression Data

In any of the examples described herein, queries can specify that the results be grouped into two or more groups by specified criteria. For example, results can be grouped into two groups: one for study subjects and the other for control subjects. If desired, any other criteria (e.g., any one or more non-gene criteria) can be used to group the results.

Having grouped the data, tools can be used to apply analyses among or between the groups. For example, cluster hierarchical analysis, Kmeans analysis, or SOM Clustering can be performed.

In this way, a researcher can investigate possible differences or correlations in gene expression between or among groups (e.g., by identifying outlier gene expression values or other phenomena).

### EXAMPLE 21

### Exemplary Implementation of Providing Results

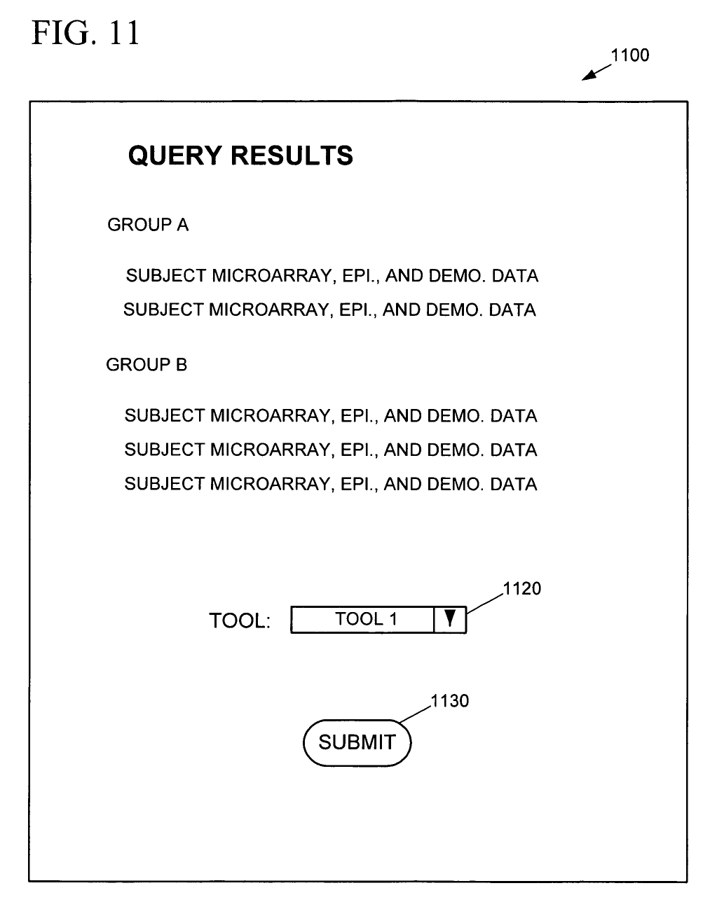

After query processing such as that described in Example 20 above, information indicating microarrays from subjects meeting the selection criteria can be displayed. FIG. 11 shows a screen shot 1100 of an exemplary user interface for displaying information indicating microarrays from subjects satisfying the criteria. Although not required, in the example, the information is grouped (e.g., according to whether the subject was a control or a case subject). Along with the microarray data, corresponding epidemiological and demographic data can be shown for the subjects meeting the criteria specified in the query. A variety of tools can be selected to further analyze the results provided (e.g., by the user interface elements 1120 and 1130).

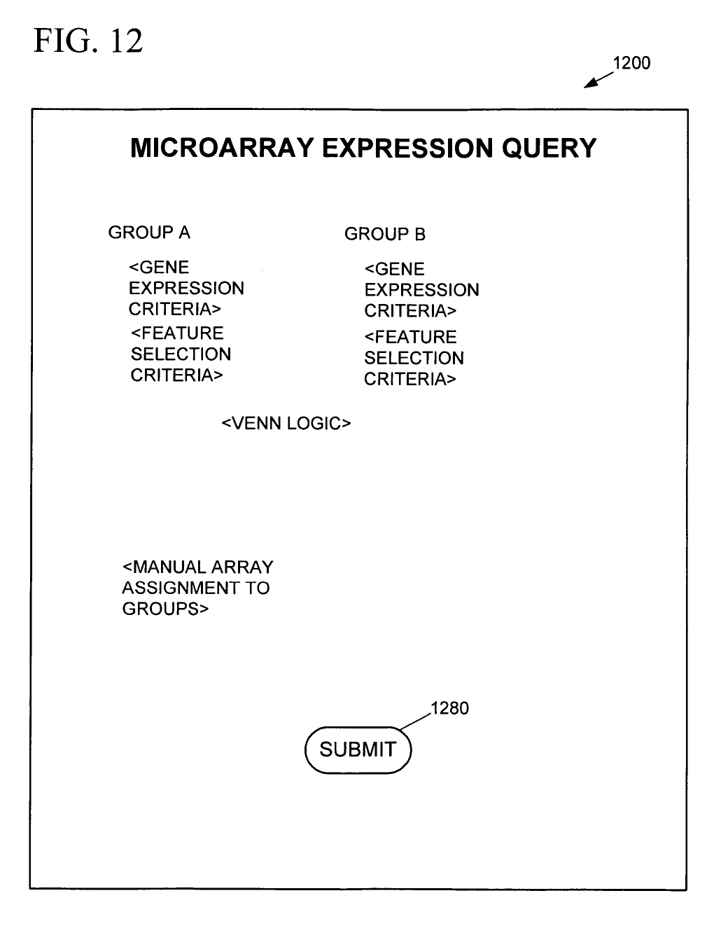

For example, one such tool allows microarray expression analysis to be performed on the microarray data. FIG. 12 shows an exemplary user interface for performing microarray expression analysis. Various spot filter options can be selected (e.g., by specifying thresholds or other criteria). Also, criteria can be specified for determining whether the set of arrays indicate a particular feature (e.g., the presence of a nucleic acid). For example, if the spot filter options result in a certain number of arrays (e.g., 2, 3, 4, or n arrays) having the feature, the feature is considered to be present in the group. VENN logic can then be applied to the presence of the features to determine similarities or differences in the group (e.g., via AND or NOT parameters). If desired, arrays can be manually moved into or out of the groups.

Upon activation of the appropriate user interface element (e.g., the pushbutton 1280), the query is processed. A display of features (e.g., by listing nucleic acid or gene names) results. The results display can identify the features (e.g., which, how many, or both) that meet the specified criteria for the groups. In addition, via the VENN logic, the results display can indicate which features satisfy criteria for one group, but not the other (or which satisfy both, if so selected).

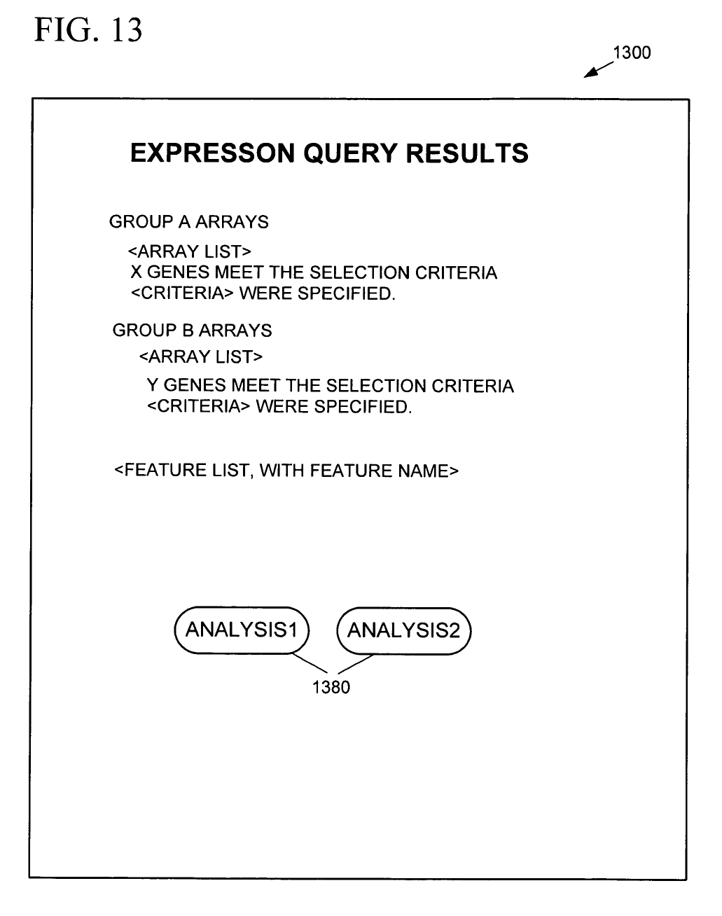

FIG. 13 shows a screen shot 1300 of an exemplary user interface for presenting the results of a microarray query, such as that in FIG. 12. Having identified the various features in the groups, visual analyses such as a hierarchical analysis, Kmeans, or SOM clustering can then be performed by activating appropriate user interface elements 1380.

### EXAMPLE 22

### Exemplary Analysis and Visualization of Retrieved Microarray Subsets

Table 1 lists exemplary retrieval and visualization tools for examining microarray data.

TABLE 1
Exemplary Tools for Analyzing Microarray Data

| Name | Description |
|---|---|
| EPI-Data Query | Selects groups of microarray experiments based on demographic and |
| | epidemiological information |
| EPI-ID Query | Selects groups of microarray experiments based on specific subject IDs |
| Ad Hoc PID Query | Provides extensive search and subsetting capabilities. For an array that satisfies |
| | a query, the experiment's image and histogram of the gene expression |
| | intensities are provided. Genes that satisfy query criteria can be clustered. |
| | Hierarchical clustering, Kmeans clustering, or Self-Organizing Maps (SOM) |
| | clustering algorithms are available. Results can be either viewed online or |
| | retrieved. |
| 1 or 2 Groups | Provides tools to compare two groups of experiments. Query conditions can |
| Logic Retrieval | be set independently for either of the two groups of arrays. Genes selected by |
| Tool (VENN | the query can be clustered. Hierarchical clustering, Kmeans clustering, and |
| Logic) | Self-Organizing Maps clustering algorithms are available. Results can be |
| | either viewed online or retrieved. |
| Scatter Plot Tool | An interactive scatter plot of gene expression intensities for any pair of |
| | experiments, allowing color-coding of gene intensities and subsetting |
| | capabilities. Available in a multi-array version whereby an array can be |
| | compared to one or more arrays |
| M v. A Plot | An interactive M v. A plot that includes LOWESS normalization and color- |
| | coding. |
| Experiment Array | For both single and multi experiments, designed for intuitively and efficiently |
| Viewer | gathering significant information from hybridization data |
| Project Summary | Display experiment data for a project (e.g., experiments in a project that meet |
| Report | specified criteria) |

Such analysis and visualization tools are available and accessible both before and after query processing. For example, the tools can be applied to a complete study (e.g., before querying takes place), or subsequent to querying (e.g., upon the results of the query). Various of the tools can be used to compare one group of microarray data to another group.

### EXAMPLE 23

### Exemplary Display of Gene Expression Data

In any of the examples described herein, a user interface can provide gene expression data (e.g., as a query result). For example, in the case of microarray data, the name of the microarray experiment can be shown. Also, icons can be provided by which an experiment's image or its histogram can be selected by activating the appropriate icon.

In the user interfaces, it is also possible to display a numerical value representing gene expression. Accompanying such a value can be the gene name, or other gene identifiers used in various databases. Upon selection of the gene name or other identifier, the user interface can navigate to an appropriate public database having information about the gene.

When displaying the gene expression data, a drop-down menu of analysis tools can be provided for initiating further examination of the results via the selected tool.

### EXAMPLE 24

### Exemplary Visualization Tool: Scatter Plot

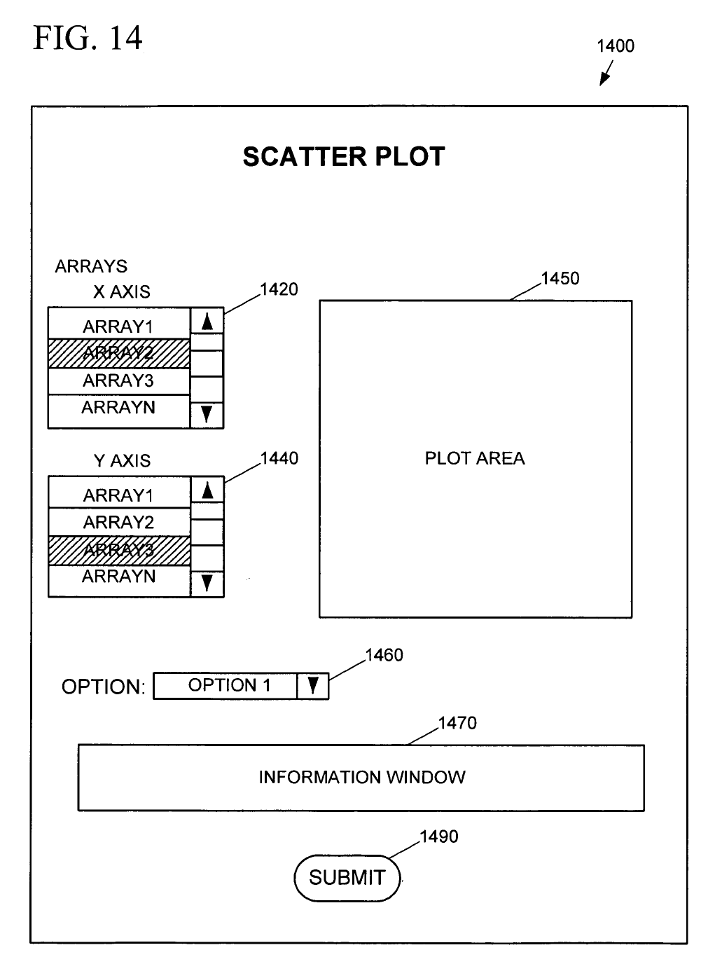

FIG. 14 shows a screen shot 1400 of an exemplary software user interface for operating a visualization tool sometimes called a “scatter plot.” In the example, the plot shows gene expression information from microarray experiments performed on samples from subjects.

In the example, a user can select an array from the list 1420 for the x-axis and an array from the list 1440 for the y-axis. The list of arrays can be arrays from a particular project (e.g., as selected in a previously displayed user interface) or a subset of them (e.g., as selected in a previously displayed user interface via specifying subject identifiers or subject criteria). If desired, control subjects can be included in the lists.

After having selected the arrays to be displayed, an appropriate scatter plot is shown in the plot area 1450 (e.g., showing gene expression information for the selected arrays as dots for a plurality of genes). In some implementations, the user clicks on a user interface element (e.g., the submit button 1490) to commence processing (e.g., generation of the scatter plot).

Various other options can be selected via user interface elements (e.g., the drop down list box 1460). For example, a minimum intensity, outlier selection criteria, intensity calculation method, and color-coding can be selected). Other information, such as correlation coefficients can be shown (e.g., Pearson or Lim's Concordance).

During operation of the user interface shown in the screen shot 1400, various information can be shown in the information window 1470. For example, when an array is selected from the lists 1420 or 1440, information related to the array (e.g., array name and description) can be shown in the window 1470. Further, when a gene is selected in the plot area 1450, information on the gene (e.g., gene id, gene name (e.g., from various public databases), gene description (e.g., from various public databases), or some combination thereof can be shown.

Further, upon selection of a gene shown in the information window 1470, the software can access one or more public databases (e.g., GenBank and the like) to generate a report (e.g., sometimes called a “feature” or “clone” report) comprising a variety of information related to the selected gene (e.g., EST's and the like) as acquired from the public database(s).

Selection of genes in the plot area 1450 can be accomplished by dragging (e.g., with a pointer device such as a mouse or trackball) over a selection area. A growable selection area thus results. Genes in the selection area are displayed in the information window 1470. If desired, the growable selection area can be configured (e.g., via a user interface element such as a radio button or checkbox) to be diagonal (e.g., at a forty five degree angle to the axis) to permit more convenient selection of outlier genes.

The example shown in FIG. 14 is for analyzing two arrays. However, a multi-array scatter plot can also be performed. For example, a 1:n arrangement can be supported wherein one array is selected for the x-axis, and a plurality of arrays are selected for the y-axis.

Further, a pairwise arrangement can be supported. In such an arrangement, an additional user interface element (e.g., a graphical pushbutton) can be shown by which a selected pair of arrays are added to the scatter plot. Any number (e.g., one or more) pairs can be added to the scatter plot in such a manner. For the pairs, a bi-variate distribution is performed.

In any of the examples described herein, color can be used in the user interface. For example, when many arrays are shown, different colors can be used to denote the different arrays. Color can also be used to indicate which genes meet specified outlier criteria.

### EXAMPLE 25

### Exemplary Visualization Tool: M v. A Plot

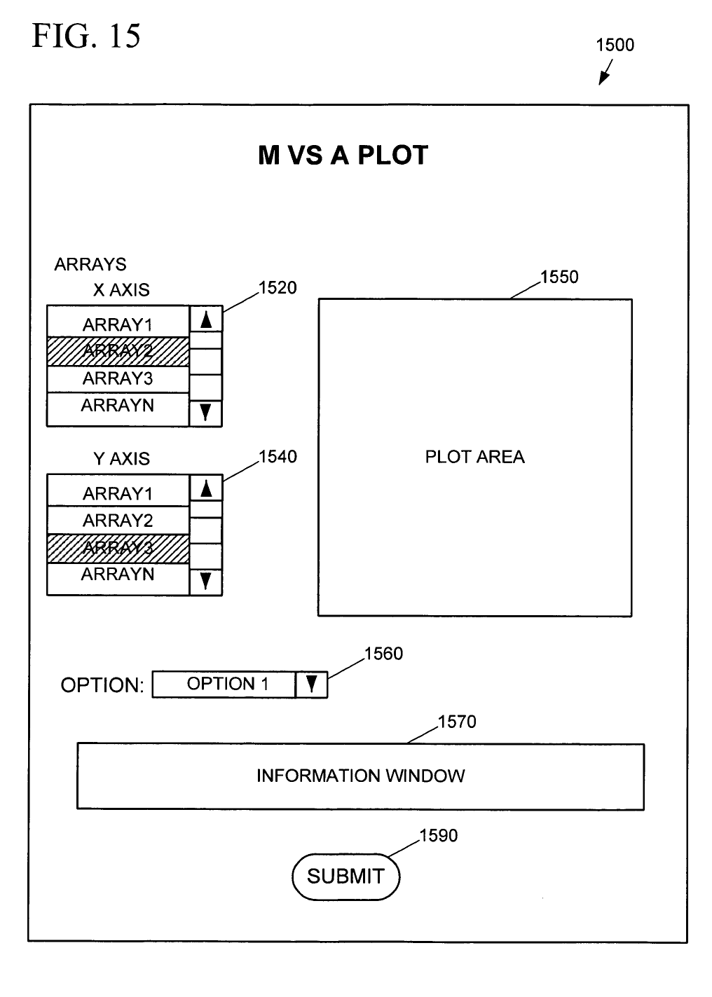

FIG. 15 shows a screen shot 1500 of an exemplary software user interface for operating a visualization tool sometimes called a “M v. A plot.” In the example, the plot shows gene expression information from microarray experiments performed on samples from subjects.

The M versus A plot computes the log intensity ratio (e.g., M=log_{—}2(R/G)) and the mean log intensity (e.g., A=log_{—}2(R*G)/2), where R and G represent the intensities of the two experiments, respectively. Logarithms base 2 can be used instead of natural or decimal logarithms because intensities are typically integers between 1 and 216. The M v. A plot allows for rapid identification of skewed data by the viewer. When plotted, the data points in a normalized set (e.g., perfectly normalized) are centered on the M=0 axis.

Microarray experiments for the x- and y-axis can be selected from the lists 1520 and 1540 (e.g., one experiment from each list).

Minimum intensities (e.g., the minimum intensity to plot) can be specified in a variety of ways. For example, a minimum intensity value can be typed into a minimum intensity field (e.g., an edit box), or a scroll bar beneath the field can be manipulated (e.g., slid via pointing device). To go beyond or below values possible with the scroll bar, the value can be typed directly into the field. The minimum intensity can be used for both experiments.

Various signal adjustment techniques can be selected via the interface. For example, data can be plotted using either raw signals (e.g., the default) or the background subtracted raw signals by manipulating a user interface element (e.g., a drop down list box).

Various signal types can be used. For example, a user interface element can be used to select Raw or Normalized intensities to draw the plot. In addition to this selection, the data can be normalized via a global Locally Weighted Scatter Plot Smoother (“LOWESS”) transformation and the LOWESS plot superimposed on the plot for the comparison purpose. The LOWESS function is a curve-fitting equation. It performs a local fit to the data in an intensity-dependent manner. The intensity value for the spots is normalized based on data distribution in the immediate neighborhood of the spot's intensity (e.g., in a limited sub-range of the intensity scale, centered on the spot's intensity value).

In order to convey additional information in the M v. A plot, data points can be color-coded based on intensity values. Because data points contains two different intensity values, a user can use a user interface element (e.g., a drop down list box 1560) to select which array to use for color-coding. The default is to use the “X axis”, which is the intensity value from the experiment specified from the “X axis” list.

In a client, server arrangement (e.g., over the Internet), a user interface element (e.g., submit button 1590) can be used to indicate that arrays have been chosen or re-chosen. Another user interface element (e.g., an “apply” button, not shown) can be used to redraw the plot area 1550 when changes to filter or outlier selections have been made.

Genes can be selected in the M v. A plot, by dragging (e.g., via a pointing device) across the genes of interest. One or more genes can be selected depending on how many points are within the dragged box. Gene information is displayed in a lower display panel (e.g., the information window 1570).

Additional information on displayed genes can be provided in a variety of ways. For example, upon selecting a text entry for a gene in the information window 1570 (e.g., via double clicking), another window (e.g., in a browser) can be opened to display additional information (e.g., links to public databases such as GenBank or the like, or information from such links) for the selected gene. Alternatively, upon selection of an entry and activation of a user interface element (e.g., a “Feature Report” button, not shown), the same window can be shown. If desired, the feature report can be exported for further use (e.g., in MICROSOFT EXCEL spreadsheet format).

If there are selected genes (e.g., as shown in the information windows 1570), activating a user interface element (e.g., a “Display List” button, not shown), another window (e.g., in a browser) will open display text entries for the genes, allowing easy printing of the list.

Various of the techniques for the M v. A plot (e.g., selection of maximum intensity, color-coding, and additional gene information techniques) can be applied to any of the scatter plot user interface examples described herein.

### EXAMPLE 26

### Exemplary Grouping for Visualization Tools

In any of the examples involving visualization tools, grouping by one or more criteria (e.g., epidemiological, demographic, or other non-gene criteria) can be used (e.g., in a query preceding the visualization tool) to group the data. In this way, comparisons between groups can be facilitated. For example, expression data from a first group can be shown as choices for the x-axis, and expression data from the second group can be shown as choices for the y-axis.

### EXAMPLE 27

### Exemplary Addition of Characteristics

The architecture of the system of any of the examples described herein to allow addition of additional subject characteristics (sometimes called “common data elements”). For example, additional non-gene (e.g., epidemiological, demographic, or both) criteria can be added to extend functionality.

For example, if a researcher wishes to track hair color for a study, an appropriate addition of one or more database tables columns can be performed. The structure of various other tables need not be changed. For example, when such data is acquired via a questionnaire, an appropriate question can be added to the table having questionnaire answers without modifying the structure of the table.

The user interfaces depicting the characteristics can be programmatically generated. Accordingly, addition of characteristics does not require re-programming of the system. For example, when a query user interface is shown by which the characteristic is specified as a query criterion, the user interface elements for specifying the added criteria (e.g., “black” for hair color) can be generated by code based on information stored in the database tables.

For example, in the example of hair color, the choices for hair color (e.g., “black” “blonde” “brown” “red”) can be stored in the database tables. Accordingly, when it comes time to generate the user interface elements for specifying hair color as a criterion, the software can pull the choices from the database tables and construction an appropriate user interface element (e.g., a list box) from which the user can select the desired hair color(s). In this way, the user interface need not be manually edited when new characteristics are desired.

Further, different projects can have different characteristics associated with them. In this way, the system can accommodate a wide variety of studies having different criteria.

### EXAMPLE 28

### Exemplary Implementation of Disparate Microarray Data Format Processing

The examples described herein can support storing and processing microarray data (e.g., expression information) from disparate microarray data formats. For example, some formats may be based on single intensity experiments, while others are from dual intensity experiments. Also, different software can produce different values or arrangements of values.

In an exemplary implementation of disparate microarray data format processing, the raw data coming from the software is kept in appropriate (e.g., separate) database tables. Various non-destructive normalization techniques can be performed on the data (e.g., keeping the original data as-is). Different normalization techniques can be performed on data from different formats. A user can select the normalization technique via a user interface element (e.g., a drop down menu presented when uploading the expression data to the database).

The expression data from the various experiments originating from data of different formats can be stored together (e.g., in a single table, such as the INTENSITY_ANALYSIS_DATA database table 1782, below). To facilitate comparisons between the data, a standard range (e.g., 0-100) can be used for the expression data when the data is stored together. In this way, the data can be stored in a uniform format.

Further, if desired, two different normalization techniques can be performed on the same experiment group to generate two different data sets. Both data sets can be stored under different names (e.g., different projects). The chosen normalization technique can be stored and displayed when a project summary is provided by the software.

Any of the tools described in any of the examples can be used to analyze data combined from experiments of two different formats or the same experiment normalized in two or more different ways. Analysis can be performed within or between projects.

There is no limit to the number of normalization techniques (e.g., linear and non-linear) that can be supported (e.g., via a gene of reference, finding the 50^{th} percentile, 75^{th} percentile, median, mean, standard deviations, background intensity, and the like), and new techniques can be added to the software as they emerge. The choice of normalization technique can be based on a variety of factors, including the quality of experiment, the type of array, and the type of imaging software.

Of particular interest is the ability to support both single and dual intensity arrays. Further, analysis of any gene or other nucleic acid can be supported as long as there is availability of some expression data, whatever the format.

### EXAMPLE 29

### Exemplary Database Schema

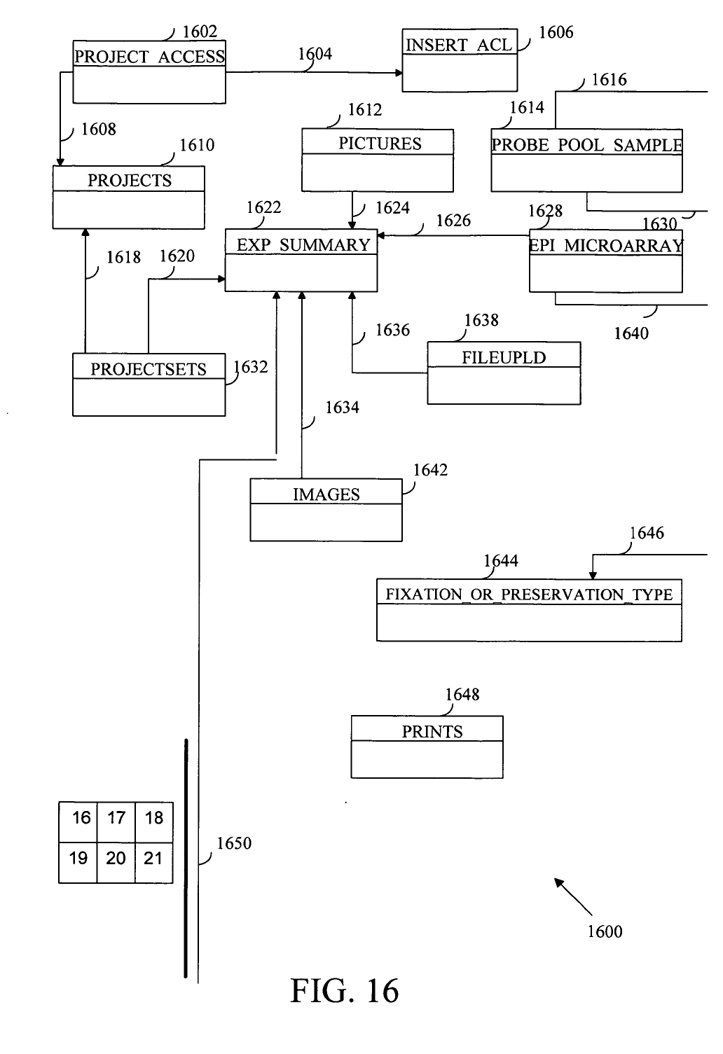

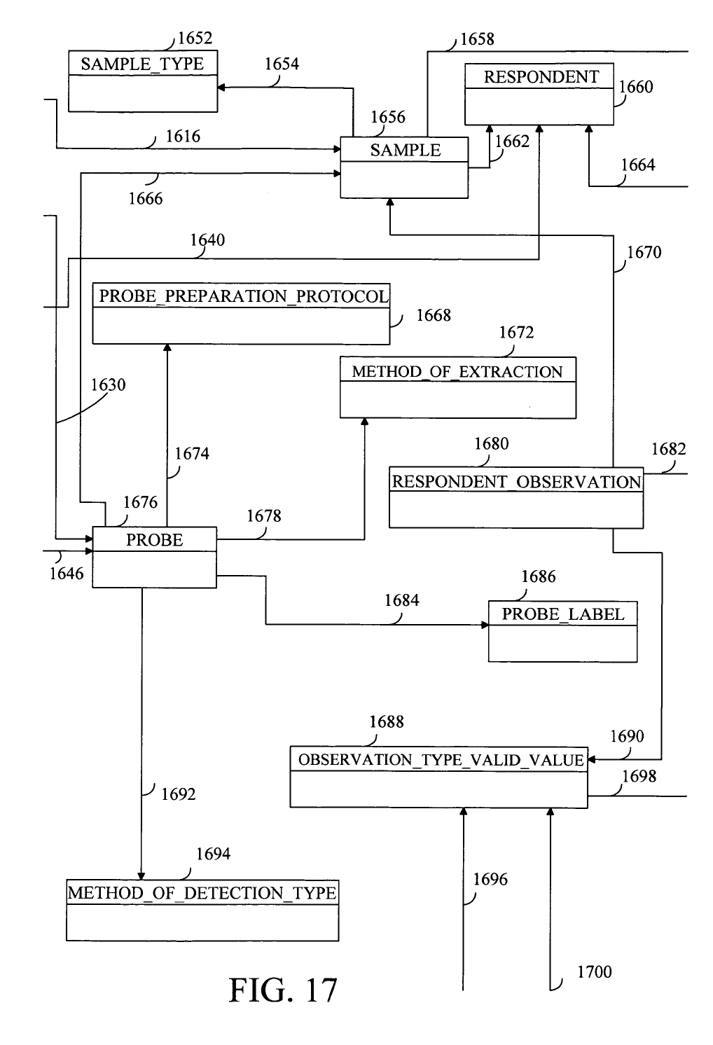

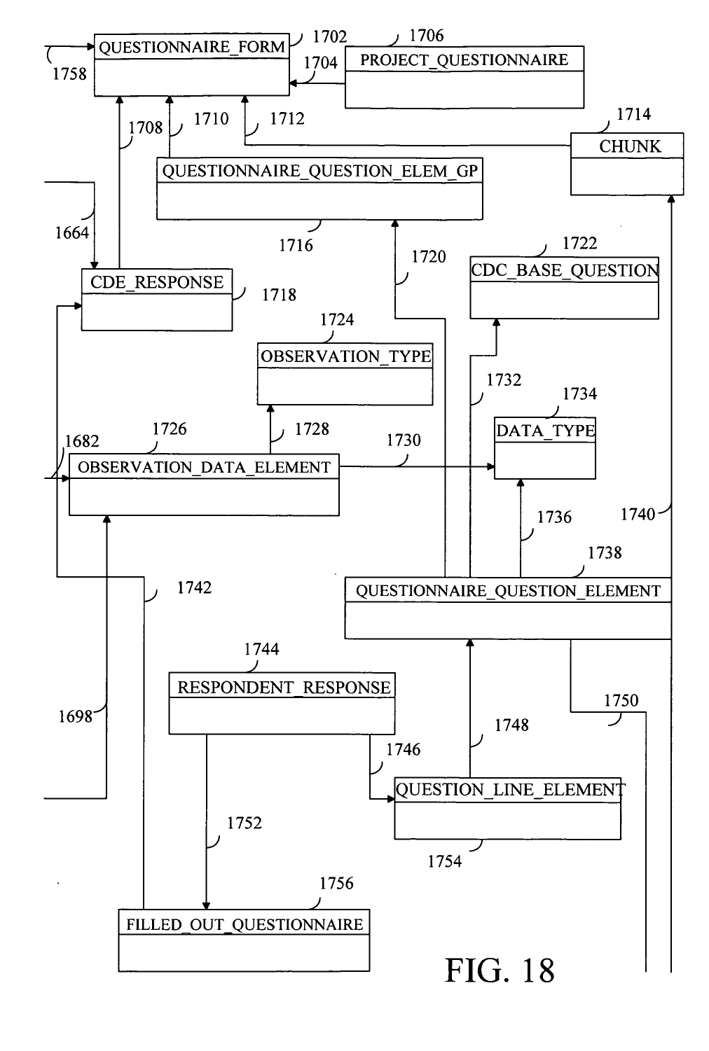

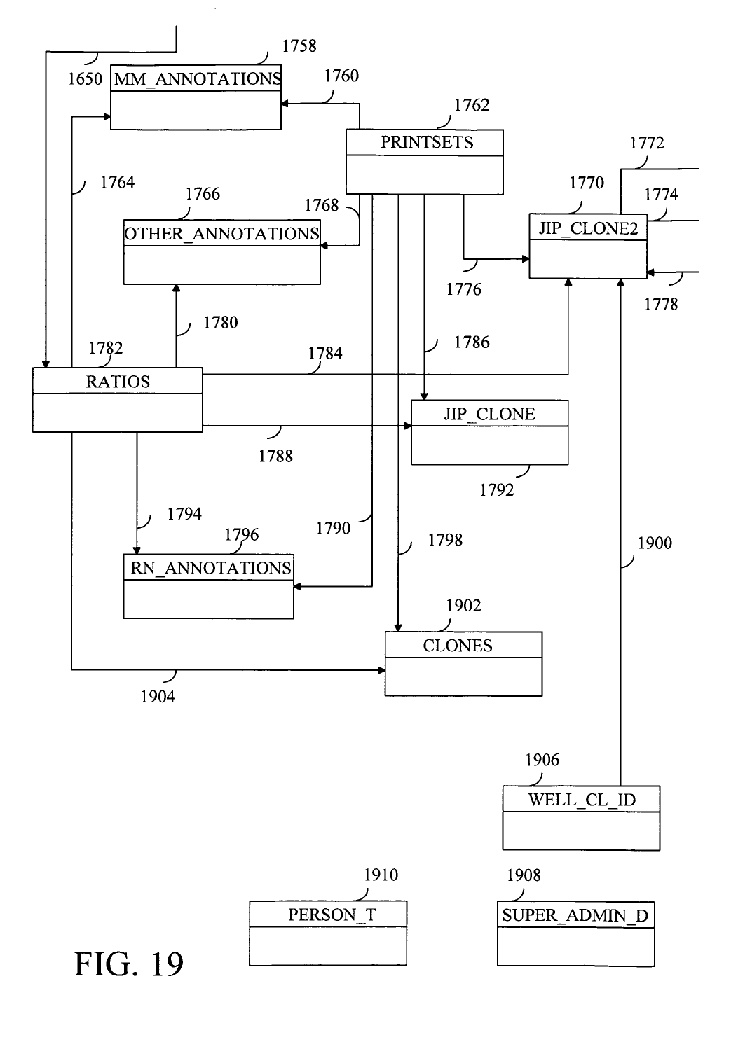

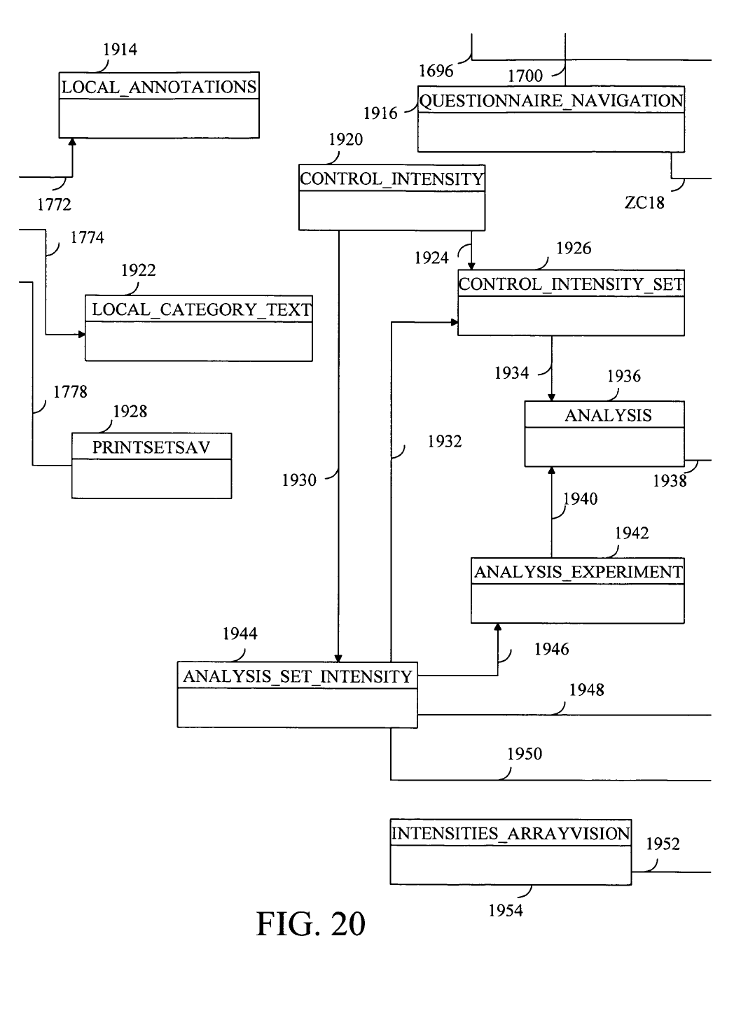

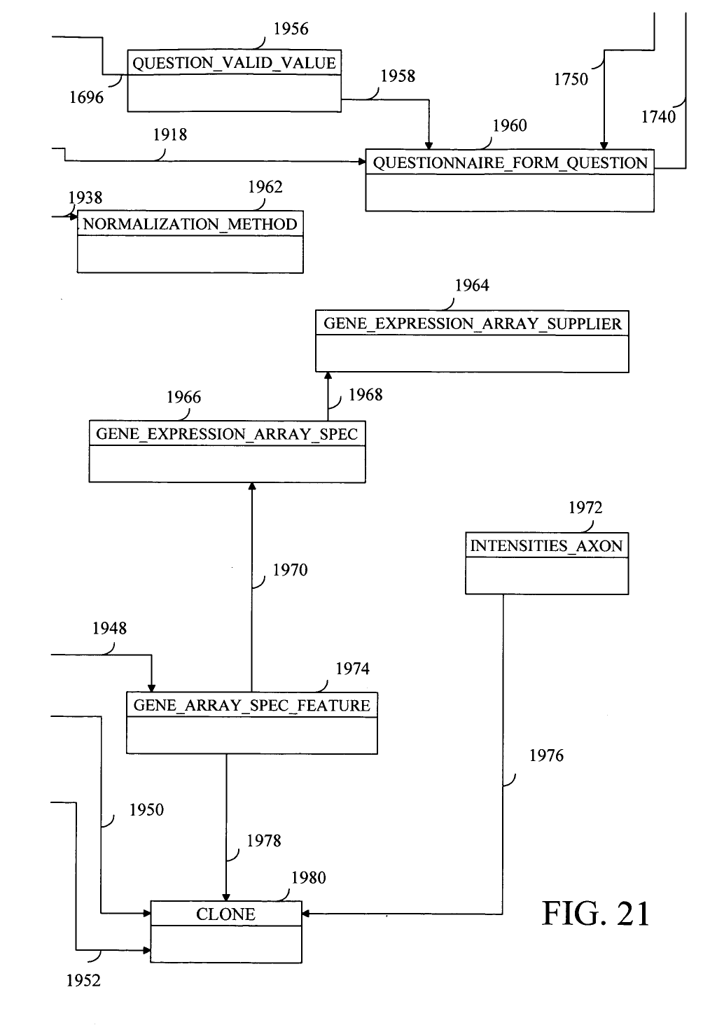

FIGS. 16, 17, 18, 19, 20, and 21 show an exemplary database schema 1600 by which the technologies described herein can be implemented.

The schema includes the database tables as shown in Table 2. Relationships between the table fields are as shown in Table 3.

TABLE 2
Database Tables

| Table Name | Fields |
|---|---|
| PROJECT_ACCESS 1602 | PROJECT_ID (Key) |
| | WWW_LOGIN (Key) |
| | UPLOAD_FLAG |
| | ADMIN_FLAG |
| | USER_IID |
| INSERT_ACL 1606 | USER_IID (Key) |
| | WWW_LOGIN |
| | EMAIL |
| | PASSWD_CHANGE_DATE |
| | PRIV_FLAG |
| | REQUEST_DATE |
| | APPROVED_DATE |
| PROJECTS 1610 | PROJECT_ID (Key) |
| | PROJECT_TYPE |
| | PROJECTNAME |
| | DESCRIPTION |
| | ENTRY_DATE |
| | ENTERED_BY |
| | COMMENTS |
| | PG1 |
| | PG2 |
| | PG3 |
| | PRINTSET_IID |
| | ARRAY_SOURCE |
| PICTURES 1612 | PIC_ID |
| | PATH |
| | FORMAT |
| | EXP_ID |
| | ARCH_FLAG |
| | SCALEFACTOR |
| | XOFFSET_PIXELS |
| | YOFFSET_PIXELS |
| PROBE_POOL_SAMPLE 1616 | SAMPLE_ID (Key) |
| | PROBE_ID (Key) |
| | RESPONDENT_ID (Key) |
| EXP_SUMMARY 1622 | EXP_ID (Key) |
| | AVG_INTENSITY1 |
| | AVG_INTENSITY2 |
| | AVG_BACKGROUND1 |
| | AVG_BACKGROUND2 |
| | AVG_SIZE1 |
| | AVG_SIZE2 |
| | SD_INTENSITY1 |
| | SD_INTENSITY2 |
| | SD_BACKGROUND1 |
| | SD_BACKGROUND2 |
| | SD_SIZE1 |
| | SD_SIZE2 |
| | CALRATIO_90MAX |
| | CALRATIO_90MIN |
| | MEAN_RATIO |
| | MEDIAN_RATIO |
| | CALIBRATION_FACTOR |
| | DBCALIBRATION_FACTOR |
| | EMPTY_SPOTS |
| | NONEMPTY_SPOTS |
| | NOTARGET_SPOTS |
| | NOTARGET1_SPOTS |
| | NOTARGET2_SPOTS |
| | CHANNEL_CD |
| EPI_MICROARRAY 1628 | PROJECT_NAME (Key) |
| | PROJECT_ID (Key) |
| | EXP_ID (Key) |
| | RESPONDENT_ID (Key) |
| PROJECTSETS 1632 | NAME |
| | EXP_ID (Key) |
| | SPOTS |
| | PRINT_IID |
| | S_DESCP |
| | C1_PROBE |
| | C2_PROBE |
| | PROJECT_ID (Key) |
| | PREFER_ORDER |
| | L_DESCP |
| | COMMENTS |
| | ID_CODE |
| | C1_PROBE_LABEL |
| | C2_PROBE_LABEL |
| | PIXEL_SIZE |
| | CALIBRATION_FACTOR |
| | C1_PROBE_ID |
| | C2_PROBE_ID |
| | PROBE_SOURCE |
| | PROBE_LABEL_METHOD |
| | NEGATIVE_CONTROL |
| | POSITIVE_CONTROL |
| | ARRAY_SOURCE |
| | MAXSIGNAL |
| | MINSIGNAL |
| | SIGNAL_CALCULATION |
| | NORMALIZATION |
| | EXCLUDE_FLAGGED_SPOTS |
| | LOT_ID |
| | SLIDE_POSITION_NUM |
| FILEUPLD 1638 | DIR_PATH (Key) |
| | UPLD_DATE |
| | PROC_DATE |
| | PROC_FLAG |
| | USER_NAME |
| | EXP_ID |
| | V_FLAG |
| | PROJECT_ID |
| IMAGES 1642 | EXP_ID |
| | SLIDE_ID |
| | RED_PROBE_ID |
| | GREEN_PROBE_ID |
| | LOW |
| | UP |
| | LOW_90 |
| | UP_90 |
| | LOW_95 |
| | UP_95 |
| | LOW_99 |
| | UP_99 |
| | LOW_100 |
| | UP_100 |
| | M |
| | CV |
| | IMAGE_ID |
| FIXATION_OR_PRESERVATION_TYPE | FIXATION_PRESERVATION_TYPE_CD |
| 1644 | FIXATION_PRESERVATION_TYPE_NM |
| PRINTS 1648 | PRINT_IID |
| | PRINT_ID (Key) |
| | PRINT_FINGERS |
| | PRINT_ROWS |
| | PRINT_COLUMNS |
| | PRINT_DATE |
| | PRINT_OPERATOR |
| | PRINTER_IID |
| | PRINT_COMMENTS |
| | PRINTSET_IID |
| | ARRAY_SOURCE |
| | ARRAY_NAME |
| SAMPLE_TYPE 1652 | SAMPLE_TYPE_CODE (Key) |
| | SAMPLE_TYPE_NAME |
| SAMPLE 1656 | RESPONDENT_ID (Key) |
| | SAMPLE_ID (Key) |
| | SAMPLE_TYPE_CODE |
| | QUESTIONNAIRE_ID |
| | DATE_FILLED_OUT |
| | SAMPLE_DATE |
| RESPONDENT 1660 | RESPONDENT_ID (Key) |
| | CASE_RESPONDENT_ID |
| | RESPONDENT_NAME |
| | CASE_OR_CONTROL_FLAG |
| PROBE_PREPARATION_PROTOCOL 1668 | PROBE_PREPARATION_PROTOCOL_CD |
| | (Key) |
| | PROBE_PREPARATION_PROTOCOL_NM |
| | PROBE_PREPARATION_PROTOCOL_DESC |
| METHOD_OF_EXTRACTION 1672 | METHOD_OF_EXTRACTION_TYPE_CODE |
| | (Key) |
| | METHOD_OF_EXTRACTION_TYPE_NAME |
| PROBE 1676 | PROBE_ID (Key) |
| | PROBE_PREPARATION_PROTOCOL_CD |
| | SAMPLE_ID |
| | FIXATION_PRESERVATION_TYPE_CD |
| | METHOD_OF_DETECTION_TYPE_CODE |
| | METHOD_OF_EXTRACTION_TYPE_CODE |
| | RESPONDENT_ID |
| | PROBE_LABEL_CODE |
| | PROBE_NAME |
| | POOLED_SAMPLE_PROBE_FLAG |
| | PROBE_SOURCE |
| | AMOUNT_OF_RNA_USED |
| | METHOD_OF_PROCUREMENT |
| RESPONDENT_OBSERVATION 1680 | RESPONDENT_ID (Key) |
| | OBSERVATION_TYPE_ID (Key) |
| | OBSERVATION_ELEMENT_ID (Key) |
| | VALID_VALUE_ID |
| | SAMPLE_ID |
| | DATE_OBSERVATION |
| | NUMERIC_OBSERVATION |
| | TEXT_OBSERVATION |
| | INTEGER_OBSERVATION |
| PROBE_LABEL 1686 | PROBE_LABEL_CODE (Key) |
| | PROBE_LABEL_NAME |
| OBSERVATION_TYPE_VALID_VALUE 1688 | VALID_VALUE_ID (Key) |
| | OBSERVATION_ELEMENT_ID |
| | OBSERVATION_TYPE_ID |
| | VALID_VALUE_CODE |
| | VALID_VALUE_TEXT |
| METHOD_OF_DETECTION_TYPE 1694 | METHOD_OF_DETECTION_TYPE_CODE |
| | (Key) |
| | METHADONE_DETECTION_TYPE_NAME |
| QUESTIONNAIRE_FORM 1702 | QUESTIONNAIRE_ID (Key) |
| | QUESTIONNAIRE_NAME |
| | QUESTIONNAIRE_DATE |
| | QUESTIONNAIRE_OWNER |
| PROJECT_QUESTIONNAIRE 1706 | PROJECT_ID (Key) |
| | QUESTIONNAIRE_ID (Key) |
| CHUNK 1714 | QUESTIONNAIRE_ID (Key) |
| | CHUNK_ID (Key) |
| | CHUNK_LABEL |
| QUESTIONNAIRE_QUESTION_ELEM_GP | QUESTIONNAIRE_ID (Key) |
| 1716 | QUESTION_ELEMENT_GROUP_ID (Key) |
| | CHUNK_ID |
| | QUESTION_GROUP_LABEL |
| | RESPONSES_ALLOWED_PER_GROUP |
| CDE_RESPONSE 1718 | QUESTIONNAIRE_ID (Key) |
| | RESPONDENT_ID (Key) |
| | CASE_OR_CONTROL |
| | DATE_OF_BIRTH |
| | GENDER |
| | BMI |
| | RACE |
| | ONSET_TYPE |
| | FATIGUE_DURATION |
| | SYMPTOMS |
| | SAMPLE-DATE |
| CDC_BASE_QUESTION 1722 | CDC_BASE_QUESTION_ID (Key) |
| | CDC_BASE_QUESTION_TEXT |
| OBSERVATION_TYPE 1724 | OBSERVATION_TYPE_ID (Key) |
| | OBSERVATION_LABEL |
| | OBSERVATION_TYPE_NAME |
| OBSERVATION_DATA_ELEMENT 1725 | OBSERVATION_TYPE_ID (Key) |
| | OBSERVATION_ELEMENT_ID (Key) |
| | DATA_TYPE_CODE |
| | OBSERVATION_ELEMENT_DATA_TYPE |
| | OBSERVATION_ELEMENT_LABEL |
| DATA_TYPE 1734 | DATA_TYPE_CODE (Key) |
| QUESTIONNAIRE_QUESTION_ELEMENT | QUESTIONNAIRE_ID (Key) |
| 1738 | QUESTION_ID (Key) |
| | QUESTION_ELEMENT_ID (Key) |
| | CHUNK_ID (Key) |
| | QUESTION_ELEMENT_GROUP_ID |
| | CDC_BASE_QUESTION_ID |
| | DATA_TYPE_CODE |
| | QUESTION_ELEMENT_LABEL |
| RESPONDENT_RESPONSE 1744 | QUESTIONNAIRE_ID (Key) |
| | RESPONDENT_ID (Key) |
| | QUESTION_LINE_ELEMENT_ID (Key) |
| | DATE_RESPONSE |
| | NUMERIC_RESPONSE |
| | TEXT_RESPONSE |
| | INTEGER_RESPONSE |
| | VALID_VALUE_RESPONSE_ID |
| | QUESTION_ID |
| | QUESTION_ELEMENT_ID |
| | DATE_FILLED_OUT (Key) |
| QUESTION_LINE_ELEMENT 1754 | QUESTION_LINE_ELEMENT_ID (Key) |
| | QUESTION_ID |
| | QUESTION_RESPONSE_LINE_NUMBER |
| | QUESTION_ELEMENT_ID |
| | QUESTIONNAIRE_ID |
| | CHUNK_ID |
| | CDC_QUESTION_ELEMENT_ID |
| | QUESTION_ELEMENT_DATA_TYPE |
| FILLED_OUT_QUESTIONNAIRE 1756 | QUESTIONNAIRE_ID (Key) |
| | RESPONDENT_ID (Key) |
| | DATE_FILLED_OUT (Key) |
| | INTERVIEWER |
| MM_ANNOTATIONS 1758 | CL_ID (Key) |
| | CLONE |
| | A_NG_ACC |
| | A_S_TITLE |
| | A_NG_TITLE |
| PRINTSETS 1762 | PRINTSET_IID (Key) |
| | SPOT_ID (Key) |
| | CL_ID |
| | CLONE |
| | PP_PLATE |
| | PP_ROW |
| | PP_COLUMN |
| | PP_PRC |
| | PI_IDENTIFIER |
| | PI_PLATE |
| | PI_ROW |
| | PI_COLUMN |
| | PI_PRC |
| | SLIDE_BLOCK |
| | SLIDE_ROW |
| | SLIDE_COLUMN |
| | PI_WELLID |
| OTHER_ANNOTATIONS 1766 | CL_ID (Key) |
| | CLONE |
| | A_NG_ACC |
| | A_S_TITLE |
| | A_NG_TITLE |
| | GSYMB |
| | CYMAP |
| JIP_CLONE2 1770 | ORG_IID |
| (Can alternatively be called | DBEST_LIBID |
| “CLONE_DETAILS”) | UGLIB_ID |
| | CL_ID (Key) |
| | CLONE |
| | ACC3 |
| | ACC5 |
| | CLUST3 |
| | CLUST_ID3 |
| | TITLE3 |
| | GSYMB3 |
| | CYMAP3 |
| | SA_PID3 |
| | LA_PID3 |
| | LA_PID3_ID |
| | CLUST5 |
| | CLUST_ID5 |
| | TITLE5 |
| | GSYMB5 |
| | CYMAP5 |
| | SA_PID5 |
| | LA_PID5 |
| | LA_PID5_ID |
| | GI3 |
| | GI5 |
| | INSERTSIZE3 |
| | INSERTSIZE5 |
| | SEQVERIFIED |
| | A_PID_INDEX |
| | GENECARD3 |
| | GENECARD5 |
| | LOCUSLINK3 |
| | LOCUSLINK5 |
| | CHROMOSOME3 |
| | CHROMOSOME5 |
| RATIOS 1782 (Can alternatively be called | EXP_ID (Key) |
| INTENSITY_ANALYSIS_DATA because it can | SPOT_ID (Key) |
| store data for single as well as dual intensity | CL_ID |
| microarray experiments) | TOP |
| | LEFT |
| | BOTTOM |
| | RIGHT |
| | BKG_MEAN_R |
| | BKG_MEAN_G |
| | BKG_DEV_R |
| | BKG_DEV_G |
| | SAMPLE_TOTAL_R |
| | SAMPLE_TOTAL_G |
| | SAMPLE_MEAN_R |
| | SAMPLE_MEAN_G |
| | SAMPLE_DEV_R |
| | SAMPLE_DEV_G |
| | SAMPLE_SIZE_R |
| | SAMPLE_SIZE_G |
| | RATIO |
| | CAL_RATIO |
| | CAL_RATIO_DB |
| | FLAG |
| | CAL_RATIO_DB1 |
| | WELLID |
| JIP_CLONE 1792 | CL_ID (Key) |
| | CLONE |
| | CLUST |
| | TITLE |
| | GSYMB |
| | CYMAP |
| | TITLE_SRC |
| | GENBANK_ID |
| | LOCUSLINK_ID |
| | SWISSPROT_ID |
| | GENE_ID |
| RN_ANNOTATIONS 1796 | CL_ID (Key) |
| | CLONE |
| | A_S_TITLE |
| | A_NG_TITLE |
| CLONES 1902 | CL_ID (Key) |
| | CLONE |
| | FLAGS |
| | SUBSET |
| WELL_CL_ID 1906 | LCWELLID (Key) |
| | CL_ID |
| SUPER_ADMIN_D 1908 | USER_ACCESS_IID (Key) |
| PERSON_T 1910 | PERSON_IID (Key) |
| | LAST_NAME |
| | FIRST_NAME |
| | MIDDLE_INITIAL |
| | HONORIFIC |
| | TITLE |
| | AFFILIATION |
| | EMAIL_ADDRESS |
| | PHONE_NBR |
| | FAX_NBR |
| | COMMENTS |
| | ADDRESS |
| | SUBSCRIBED_FLAG |
| | UPDATE_DATE |
| LOCAL_ANNOTATIONS 1914 | A_PID_INDEX (Key) |
| | A_S_TITLE |
| | A_NG_ACC |
| | A_NG_TITLE |
| | A_CATEGORY |
| QUESTIONNAIRE_NAVIGATION 1916 | QUESTIONNAIRE_ID (Key) |
| | QUESTION_ID (Key) |
| | QUESTION_ELEMENT_ID (Key) |
| | VALID_VALUE_ID (Key) |
| | NEXT_QUESTION_ID |
| | CHUNK_ID |
| | QUE_CHUNK_ID |
| CONTROL_INTENSITY 1920 | ANALYSIS_ID (Key) |
| | EXPERIMENT_ID (Key) |
| | GENE_EXPRESSION_ARRAY_ID (Key) |
| | FEATURE_ID (Key) |
| | CLONE_ID (Key) |
| | CONTROL_SET_ID (Key) |
| LOCAL_CATEGORY_TEXT 1922 | A_PID_INDEX (Key) |
| | A_CATEGORY_TEXT |
| CONTROL_INTENSITY_SET 1925 | CONTROL_SET_ID (Key) |
| | ANALYSIS_ID |
| PRINTSETSAV 1928 | PRINTSET_IID (Key) |
| | SPOT_ID |
| | CL_ID |
| | CLONE |
| | PP_PLATE |
| | PP_ROW |
| | PP_COLUMN |
| | PP_PRC |
| | PI_IDENTIFIER (Key) |
| | PI_PLATE |
| | PI-ROW |
| | PI_COLUMN |
| | PI_PRC |
| | SLIDE_BLOCK |
| | SLIDE_ROW |
| | SLIDE_COLUMN |
| | PI_WELLID |
| ANALYSIS 1936 | ANALYSIS_ID (Key) |
| | NORMALIZATION_METHOD_CODE |
| | ANALYSIS_COMMENT |
| | ANALYSIS_DATE |
| ANALYSIS_EXPERIMENT 1942 | EXPERIMENT_ID (Key) |
| | ANALYSIS_ID (Key) |
| | NORMALIZATION_ROLE |
| ANALYSIS_SET_INTENSITY 1944 | ANALYSIS_ID (Key) |
| | EXPERIMENT_ID (Key) |
| | GENE_EXPRESSION_ARRAY_ID (Key) |
| | FEATURE_ID (Key) |
| | CLONE_ID (Key) |
| | CONTROL_SET_ID |
| | NORMALIZED_RATIO |
| | NORMALIZED_INTENSITY |
| | NORMALIZATION_ROLE |
| | RATIO |
| | CAL_RATIO |
| | CAL_RATIO_DB |
| | CAL_RATIO_DB1 |
| | CALRATIO_90MAX |
| | CALRATIO_90MIN |
| | MEAN_RATIO |
| | MEDIAN_RATIO |
| | CALIBRATION_FACTOR |
| | DBCALIBRATION_FACTOR |
| INTENSITIES_ARRAYVISION 1954 | EXP_IID (Key) |
| | GIPO_INDEX (Key) |
| | CLONE_ID |
| | FEATURE_BLOCK |
| | FEATURE_COL |
| | FEATURE_ROW |
| | FEATURE_CENTER_X_COORD |
| | FEATURE_CENTER_Y_COORD |
| | FEATURE_AREA |
| | ARM_DENSITY_MEAN |
| | PERCENTAGE_REMOVED |
| | MAD_LEVELS |
| | SD_LEVELS |
| | BKG_MEDIAN |
| | SARMDENSITY |
| | RATIO_S_N |
| | QUALITY_FLAG |
| | PCT_AT_FLOOR |
| | PCT_AT_CEILING |
| | PCT_AT_FLOOR_MINUS_BKG |
| | PCT_AT_CEILING_MINUS_BKG |
| | WELLID |
| QUESTION_VALID_VALUE 1956 | QUESTIONNAIRE_ID (Key) |
| | QUESTION_ID (Key) |
| | QUESTION_ELEMENT_ID (Key) |
| | VALID_VALUE_ID (Key) |
| | CHUNK_ID |
| | QUE_CHUNK_ID |
| | VALID_VALUE_CODE |
| | VALID_VALUE_TEXT |
| QUESTIONNAIRE_FORM_QUESTION 1960 | QUESTIONNAIRE_ID (Key) |
| | QUESTION_ID (Key) |
| | CHUNK_ID (Key) |
| | FORM_QUESTION_NUMBER |
| | QUESTION_LABEL |
| | QUESTION_TEXT |
| | NUMBER_OF_RESPONSE_GROUPS |
| NORMALIZATION_METHOD 1962 | NORMALIZATION_METHOD_CODE (Key) |
| | NORMALIZATION_METHOD_DESC |
| GENE_EXPRESSION_ARRAY_SUPPLIER | GENE_EXPRSN_ARRAY_SPLIER_CODE |
| 1964 | (Key) |
| | GENE_EXPRSN_ARRAY_SPLIER_NAME |
| | GENE_EXPRSN_ARRAY_SPLIER_PHONE |
| GENE_EXPRESSION_ARRAY_SPEC 1966 | GENE_EXPRESSION_ARRAY_SPEC_ID |
| | (Key) |
| | GENE_EXPRSN_ARRAY_SPLIER_CODE |
| | GENE_EXPRESSION_ARRAY_MFGR_CD |
| | GENE_EXPRSN_ARRAY_PRINTER_CODE |
| | GENE_EXPRESSION_ARRAY_NAME |
| | VENDOR_CATALOG_NUMBER |
| GENE_ARRAY_SPEC_FEATURE 1974 | FEATURE_ID (Key) |
| | CLONE_ID (Key) |
| | GENE_EXPRESSION_ARRAY_SPEC_ID |
| | BLOCK_ROW |
| | BLOCK_COLUMN |
| | FEATURE_ROW |
| | FEATURE_COLUMN |
| INTENSITIES_AXON 1972 | EXP_IID (Key) |
| | GIPO_INDEX (Key) |
| | CLONE_ID |
| | FEATURE_BLOCK |
| | FEATURE_COL |
| | FEATURE_ROW |
| | FEATURE_CENTER_X_COORD |
| | FEATUER_CENTER_Y_COORD |
| | FEATURE_DIAMETER |
| | CH1_FEATURE_MEDIAN |
| | CH1_FEATURE_MEAN |
| | CH1_FEATURE_SD |
| | CH1_BK_MEDIAN |
| | CH1_BK_MEAN |
| | CH1_BK_SD |
| | CH1_PCT_GT_ONE_SD |
| | CH1_PCT_GT_TWO_SD |
| | CH1_FEATURE_PCT_SATURATION |
| | CH2_FEATURE_MEDIAN |
| | CH2_FEATURE_MEAN |
| | CH2_FEATURE_SD |
| | CH2_BK_MEDIAN |
| | CH2_BK_MEAN |
| | CH2_BK_SD |
| | CH2_PCT_GT_ONE_SD |
| | CH2_PCT_GT_TWO_SD |
| | CH2_FEATURE_PCT_SATURATION |
| | RATIO_OF_MEDIANS |
| | RATIO_OF_MEANS |
| | MEDIAN_OF_RATIOS |
| | MEAN_OF_RATIOS |
| | RATIOS_SD |
| | REGRESSION_RATIO |
| | REGRESSION_RATIO_SQUARED |
| | FEATURE_PIXELS |
| | BK_PIXELS |
| | SUM_OF_MEDIANS |
| | SUM_OF_MEANS |
| | LOG_RATIO_OF MEDIANS |
| | CH1-MEDIAN_MINUS_BK |
| | CH2_MEDIAN_MINUS_BK |
| | CH1_MEAN_MINUS_BK |
| | CH2_MEAN_MINUS_BK |
| | QUALITY_FLAG |
| | WELLID |
| CLONE 1980 | CLONE_ID (Key) |
| | CLONE_NAME |
| | CLONE_DESCRIPTIVE_TEXT |
| | ACCESSION_NUMBER |
| | UNIGENE_ID |
| | GENE_CARDS_ID |
| | SEQUENCE |

TABLE 3
Database Table Relationships

| Relationship | Field(s) Relating Database Tables |
|---|---|
| 1604 | USER_IID |
| 1608 | PROJECT_ID |
| 1616 | RESPONDENT_ID, SAMPLE_ID |
| 1618 | PROJECT_ID |
| 1620 | EXP_ID |
| 1624 | EXP_ID |
| 1625 | EXP_ID |
| 1630 | PROBE_ID |
| 1634 | EXP_ID |
| 1636 | EXP_ID |
| 1640 | RESPONDENT_ID |
| 1646 | FIXATION_PRESERVATION_TYPE_CD |
| 1650 | EXP_ID |
| 1654 | SAMPLE_TYPE_CODE |
| 1658 | QUESTIONNAIRE_ID |
| 1662 | RESPONDENT_ID |
| 1664 | RESPONDENT_ID |
| 1666 | RESPONDENT_ID, SAMPLE_ID |
| 1670 | RESPONDENT_ID, SAMPLE_ID |
| 1674 | PROBE_PREPARATION_PROTOCOL_CD |
| 1678 | METHOD_OF_EXTRACTION_TYPE_CODE |
| 1682 | OBSERVATION_TYPE_ID, |
| | OBSERVATION_ELEMENT_ID |
| 1684 | PROBE_LABEL_CODE |
| 1690 | VALID_VALUE_ID |
| 1692 | METHOD_OF_DETECTION_TYPE_CODE |
| 1696 | VALID_VALUE_ID |
| 1698 | OBSERVATION_TYPE_ID, |
| | OBSERVATION_ELEMENT_ID |
| 1700 | VALID_VALUE_ID |
| 1704 | QUESTIONNAIRE_ID |
| 1708 | QUESTIONNAIRE_ID |
| 1710 | QUESTIONNAIRE_ID |
| 1712 | QUESTIONNAIRE_ID |
| 1720 | QUESTIONNAIRE_ID, |
| | QUESTION_ELEMENT_GROUP_ID |
| 1728 | OBSERVATION_TYPE_ID |
| 1730 | DATA_TYPE_CODE |
| 1732 | CDC_BASE_QUESTION_ID |
| 1736 | DATA_TYPE_CODE |
| 1740 | QUESTIONNAIRE_ID, CHUNK_ID |
| 1742 | QUESTIONNAIRE_ID, RESPONDENT_ID |
| 1746 | QUESTION_LINE_ELEMENT_ID |
| 1748 | QUESTIONNAIRE_ID, QUESTION_ID, |
| | QUESTION_ELEMENT_ID, CHUNK_ID |
| 1750 | QUESTIONNAIRE_ID, QUESTION_ID, |
| | CHUNK_ID |
| 1752 | QUESTIONNAIRE_ID, RESPONDENT_ID, |
| | DATE_FILLED_OUT |
| 1760 | CL_ID |
| 1764 | CL_ID |
| 1768 | CL_ID |
| 1772 | A_PID_INDEX |
| 1774 | A_PID_INDEX |
| 1776 | CL_ID |
| 1778 | CL_ID |
| 1780 | CL_ID |
| 1784 | CL_ID |
| 1786 | CL_ID |
| 1788 | CL_ID |
| 1790 | CL_ID |
| 1794 | CL_ID |
| 1798 | CL_ID |
| 1900 | CL_ID |
| 1904 | CL_ID |
| 1918 | QUESTIONNAIRE_ID, QUESTION_ID, |
| | CHUNK_ID |
| 1924 | CONTROL_SET_ID |
| 1930 | ANALYSIS_ID, EXPERIMENT_ID, |
| | GENE_EXPRESSION_ARRAY_ID, |
| | FEATURE_ID, CLONE_ID |
| 1932 | CONTROL_SET_ID |
| 1934 | ANALYSIS_ID |
| 1938 | NORMALIZATION_METHOD_CODE |
| 1940 | ANALYSIS_ID |
| 1946 | EXPERIMENT_ID, ANALYSIS_ID |
| 1948 | FEATURE_ID, CLONE_ID |
| 1950 | CLONE_ID |
| 1952 | CLONE_ID |
| 1958 | QUESTIONNAIRE_ID, QUESTION_ID, |
| | CHUNK_ID |
| 1968 | GENE_EXPRSN_ARRAY_SPLIER_CODE |
| 1970 | GENE_EXPRESSION_ARRAY_SPEC_ID |
| 1976 | CLONE_ID |
| 1978 | CLONE_ID |

In the example, various linking mechanisms are provided. For example, the EPI_MICROARRAY database table serves as a linking table to link non-gene and gene expression information, as do the fields within the table.

Further, in the example, study subjects are sometimes called “respondents.”

### EXAMPLE 30

### Exemplary Epidemiological Data Tables

Various of the tables can store epidemiological data. For example, in the schema of Example 29, the database tables shown in Table 4 store epidemiological data.

TABLE 4
Epidemiological Database Tables
Table Name

QUESTIONNAIRE_FORM
RESPONDENT
CDE_RESPONSE
CDC_BASE_QUESTION
OBSERVATION_TYPE
DATA_TYPE
CHUNK
QUESTIONNAIRE_QUESTION_ELEM_GP
QUESTIONNAIRE_FORM_QUESTION
QUESTIONNAIRE_QUESTION_ELEMENT
QUESTION_LINE_ELEMENT
QUESTION_VALID_VALUE
FILLED_OUT_QUESTIONNAIRE
SAMPLE
OBSERVATION_DATA_ELEMENT
OBSERVATION_TYPE_VALID_VALUE
PROBE_POOL_SAMPLE
RESPONDENT_RESPONSE
QUESTIONNAIRE_NAVIGATION
RESPONDENT_OBSERVATION

The PROJECT_QUESTIONNAIRE table can serve as a link between an epidemiological questionnaire and a microarray project data set. The CDE_RESPONSE table contains common data elements extracted from the data entered in the RESPONDENT_RESPONSE and RESPONDENT_OBSERVATION tables. The EPI_MICROARRAY table is the key table that stores the PROJECT_NAME, PROJECT_ID, EXP_ID, and the RESPONDENT_ID. EXP_ID is the identifier used on the microarray side of the schema, and the RESPONDENT_ID is its counterpart on the epidemiological side of the database. The EXP_ID column is also stored in the microarray table PROJECTSETS.

The data in the tables can be acquired in many ways (e.g., via user interfaces or by tools parsing a data source such as a spreadsheet).

### EXAMPLE 31

### Exemplary Gene Expression Data Tables

Various tables of the database can store gene expression data (e.g., analyzed microarray experiment data). An array experiment is saved as a list of values in the database data table in addition to the information about the oligonucleotide probes used in an experiment. For example, in the schema of Example 29, the microarray data can be divided into three subgroups of database tables shown in Tables 5A, 5B, and 5C.

TABLE 5A
Microarray Experiment Tables

| Table Name | Description |
|---|---|
| PROJECTS | Contains a key (e.g., PROJECT_ID) to identify the subjects |
| | whose epidemiological information and microarray |
| | information is logically stored as a single group of array |
| | experiments. |
| PROJECTSETS | A subset of a project in which an individual array |
| | experiment record for parent projects is stored. |
| FILEUPLD | Table for file uploads |
| INTENSITIES_AXON | Stores the raw intensity data for Axon based |
| | oligonucleotide arrays |
| INTENSITIES_ARRAYVISION | Stores the raw intensity data for ArrayVision based |
| | oligonucleotide arrays. |
| PICTURES | Stores the geometry information of an array image |
| RATIOS | Stores the calibrated/normalized raw intensity values |
| EXP_SUMMARY | Stores aggregate statistics on an individual array |
| | experiment |

TABLE 5B
Microarray Print-Slide Information Tables

| Table Name | Description |
|---|---|
| PRINTS | Describes an array set in terms of the number of genes, |
| | blocks, gene mapping, array source, etc. |
| PRINTSETS | Stores the physical location of an individual gene on |
| | a glass slide with gene ID and the gene name |
| | in Axon format. |
| PRINTSETSAV | Stores the physical location of an individual gene on |
| | a glass slide with gene ID and the gene name |
| | in ArrayVision format. |
| JIP_CLONE | Stores the gene array list with associated Unigene, |
| | LocusLink, GenBank, and SWISSPROT identification |
| | numbers. |
| WELL_CL_ID | Stores Clones. |

Table 5C shows exemplary user administration database tables from the schema discussed in Example 29. Via the User Administration database Tables, access to the data can be regulated. In this way, the system can be shared by a plurality of users who can be working on various projects without allowing others outside the authorized group to have access to the data.

TABLE 5C
User Administration Tables

| Table Name | Description |
|---|---|
| INSERT_ACL | Stores an identifier that is used to grant access |
| | permission to the system (e.g., via web interface) |
| PERSON_T | Stores the details of a person whose account |
| | is being set up |
| PROJECT_ACCESS | Stores various access privileges on a project |
| SUPER_ADMIN_D | Grants system admin privileges to the user |

### EXAMPLE 32

### Exemplary Query Implementation

Queries can be implemented in the schema of Example 29. For example, in one type of query, called an “EPI-ID Query,” the table called EPI_MICROARRAY is queried for the column RESPONDENT_ID by passing in the project ID. The results from the query are shown as the subject ids in the EPI-ID Query tool. The EPI_MICROARRAY table is the key table that stores the PROJECT_NAME, PROJECT_ID, EXP_ID, and the RESPONDENT_ID. EXP_ID is the identifier used on the microarray side of the schema, and the REPONDENT_ID is its counterpart on the epidemiology data side of the database.

Once a user selects the subject IDs of interest and clicks the Submit button, the highlighted subject IDs are passed on to the database query that is composed of two tables EPI_MICROARRAY and the PROJECTSETS. This query brings back the array or experiment name and its short description that was entered by the user during the upload process. These two elements are stored in the project sets table.

As described above, the PROJECTSETS table can have the following columns: NAME, EXP_ID, SPOTS, PRINT_IID, S_DESCP, C1_PROBE, C2_PROBE, PROJECT, PREFER_ORDER, L_DESCP, COMMENTS, ID_CODE, C1_PROBE_LABEL, C2_PROBE_LABEL, PIXEL_SIZE, CALIBRATION_FACTOR, C1_PROBE_ID, C2_PROBE_ID, PROBE_SOURCE, PROBE_LABEL_METHOD, NEGATIVE-CONTROL, POSITIVE_CONTROL, ARRAY_SOURCE, MAXSIGNAL, MINSIGNAL, SIGNAL_CALCULATION, NORMALIZATION, EXCLUDE_FLAGGED_SPOTS, LOT_ID, SLIDE_POSITION_NUM)

An exemplary query is shown in Table 6.

TABLE 6
Exemplary Query against the PROJECTSETS Table

```
Select distinct EM.EXP_ID, PS.NAME, PS.S_DESC
    from EPI_MICROARRAY EM,
        PROJECTSETS PS
    where PS.EXP_ID = EM.EXP_ID
    and EM.PROJECT_ID = PROJECT_ID of the project selected from
the analysis tools page and EM.PROJECT = PS.PROJECT
    and EM.RESPONDENT_ID IN (RESPONDENT_ID highlighted
on the EPI-ID Query Tool page)
```

When the EPI-Data Query tool is launched, the list of the subject characteristics are displayed along with the list of the projects that have both epidemiological and microarray information stored in the system database. Actual values associated with these characteristics are stored in a table called CDE_RESPONSE (common data elements response).

As shown above, the CDE_RESPONSE database table has the following columns: QUESTIONNAIRE_ID, RESPONDENT_ID, CASE_OR_CONTROL, DATA_OF_BIRTH, GENDER, BMI, RACE, ONSET_TYPE, FATIGUE_DUARATION, SYMPTOMS, SAMPLE_DATE).

Once a user selects the characteristics and clicks the submit button, a query is written dynamically, based on the search options selected on the previous screen to search for possible experiment IDs that match the filtering criteria.

An exemplary query is shown in Table 7.

TABLE 7
Exemplary Query against the CDE_RESPONSE Table

```
Select distinct EM.EXP_ID, PS.NAME, PS.S_DESC
    from EPI_MICROARRAY EM,
        PROJECTSETS PS,
        CDE_RESPONSE CR,
        PROJECT_QUESTIONNAIRE PQ
    where PS.EXP_ID = EM.EXP_ID
    and PQ.QUESTIONNAIRE_ID = CR.QUESTIONNAIRE_ID
    and CR.RESPONDENT_ID = EM.RESPONDENT_ID
    and EM.PROJECT_ID = PROJECT_ID of the project selected from
the analysis tools page
    and EM.PROJECT_ID = PS.PROJECT_ID
    and the other remaining conditions based on the characteristics
    selected above.
For example, if the value selected for the element “Case/Control” is
not None, then the “and” clause would be:
    and CR.CASE_OR_CONTROL = CASECONTROL response
    from above.
```

### EXAMPLE 33

### Exemplary Operation of Exemplary Implementation

The following describes exemplary operation of an exemplary implementation of the technologies described herein. In the example, the data was collected as part of a CFS study, but the example could easily be adapted for additional or other studies. A user navigated between the depicted exemplary user interfaces via web browser software. In the examples in which a MICROSOFT EXCEL spreadsheet is shown, the data has been exported to EXCEL spreadsheet format and can be saved for further analysis in the EXCEL spreadsheet product or some other software accommodating such a format. Other formats can be supported (e.g., UNIX, a format for APPLE MACINTOSH computers, PC, and Eisen cluster).

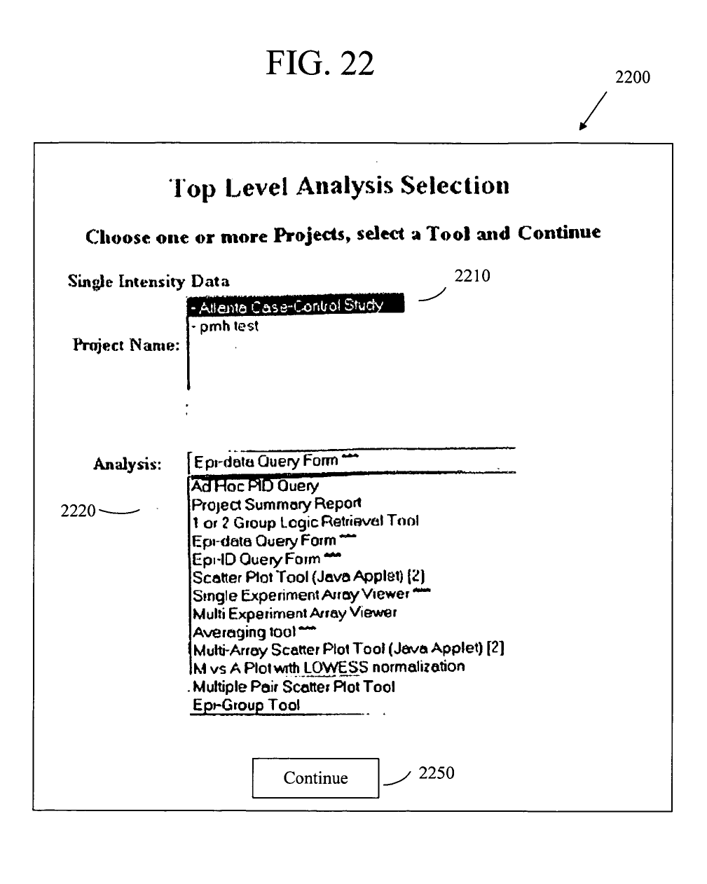

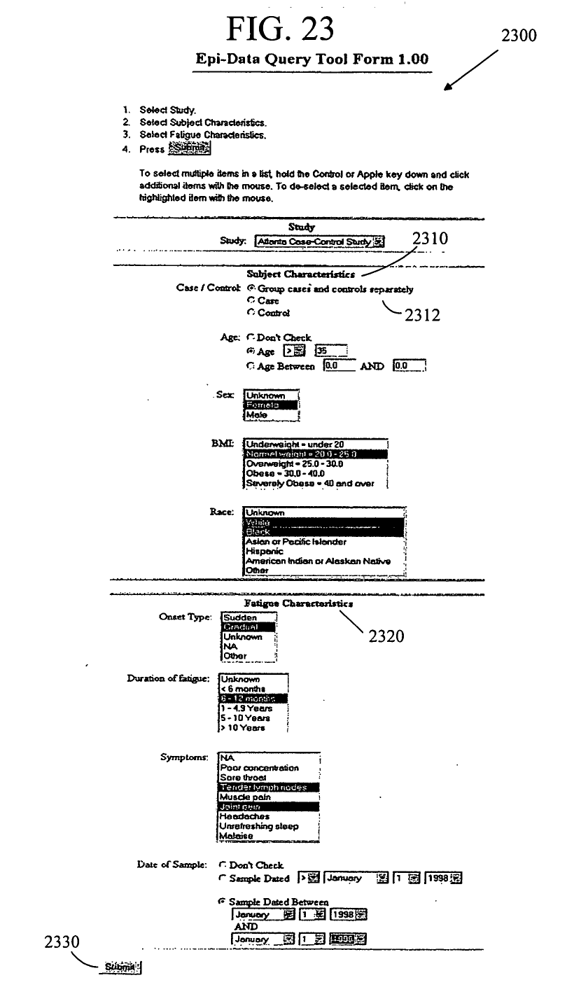

FIG. 22 shows a screen shot 2200 from the exemplary operation. The screen shot 2200 depicts a user interface by which a user can select a project and a tool. A list box 2210 shows possible choices from which a user can select a project, and a list box 2220 (e.g., an analysis tool menu) from which an appropriate analysis (e.g., tool) can be selected. In the example, the Epi-Group Tool is selected and the Continue button 2250 activated. As a result of having selected the Epi-Group Tool and activating the Continue button 2250, the screen shot 2300 of FIG. 23 is displayed.

FIG. 23 shows a screen shot 2300 displaying a user interface by which a user can indicate criteria (e.g., non-gene criteria) for a query performed on the database tables. The user can specify one or more subject characteristics (e.g., demographic characteristics) via the subject characteristics pane 2310 and one or more fatigue characteristics (e.g., epidemiological characteristics) via the fatigue characteristics pane 2320. Grouping can be accomplished by selecting “Group cases and controls separately” via the radio button 2312. When finished, the user can activate the Submit button 2330. As a result of activating the Submit button 2330, a query is performed, and microarray data associated with subjects meeting the criteria are provided (e.g., displayed) via the interface in the screen shot 2400 of FIG C(A).

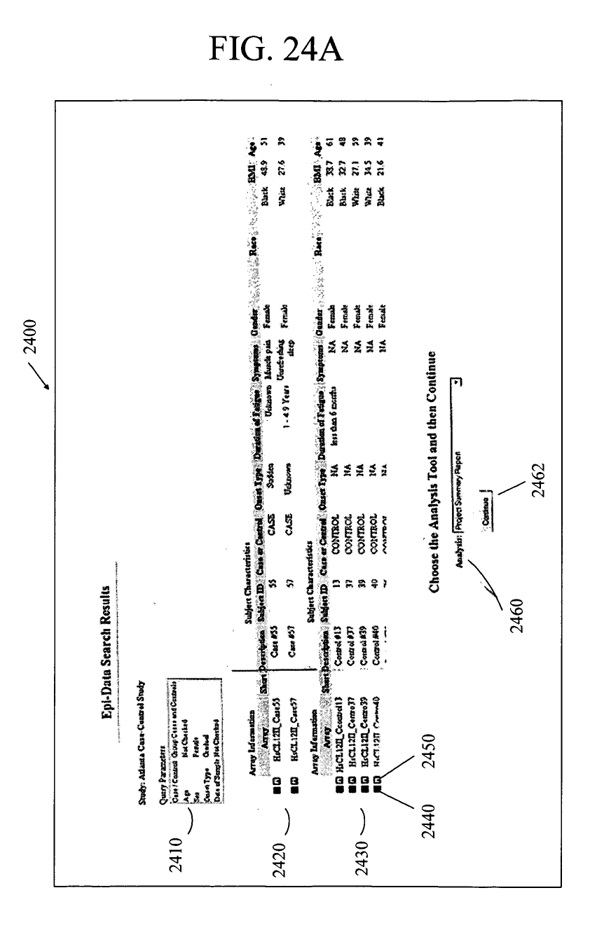

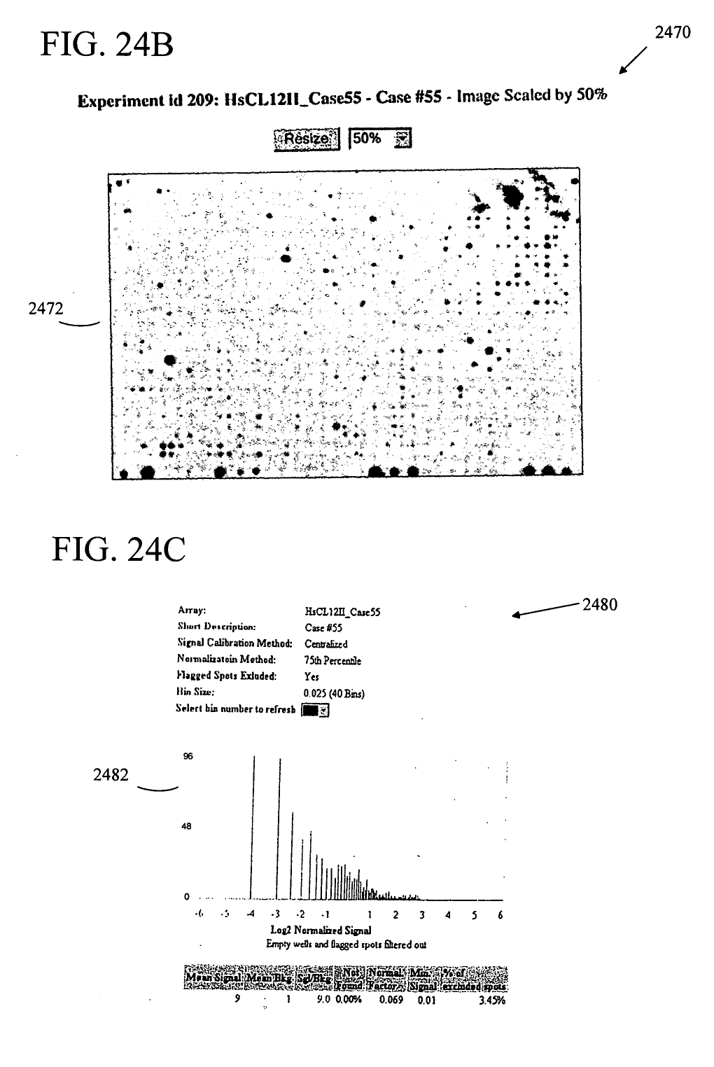

FIG. 24A shows a screen shot 2400 displaying a user interface by which query results for the criteria specified are displayed. For convenience of the user, the user interface includes the query parameters (e.g., specified criteria) 2410. The cases information 2420 (e.g., for case subjects 55 and 57) and controls information 2430 (e.g., for control subjects 13, 37, 39, etc.) are provided separately. Each line of information corresponds to a microarray experiment associated with a subject meeting the specified criteria. Various non-gene data is also shown in the line.

A button 2440 can be activated to display the microarray experiment image 2470 shown in the screen shot 2470 of FIG. 24B. Another button 2450 can be activated to display the histogram associated with the microarray as shown in the screen shot 2480 of FIG. 24C.

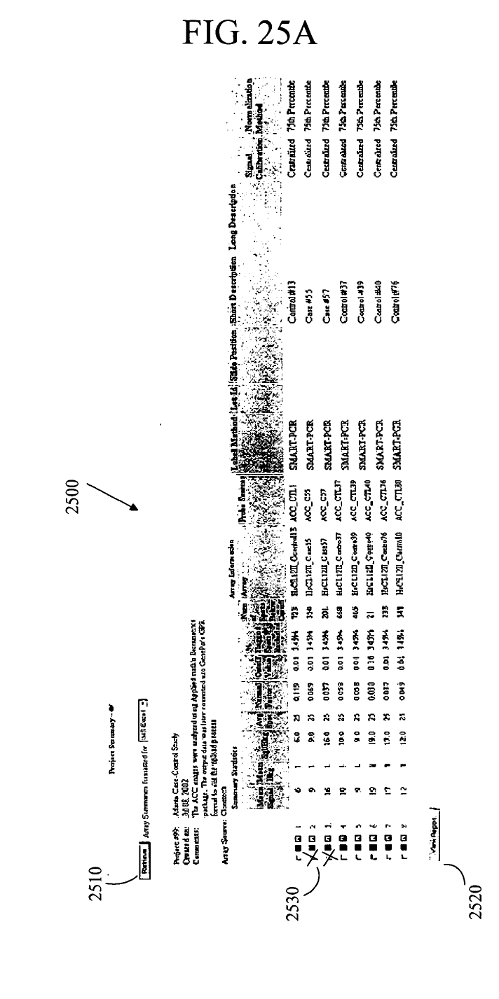

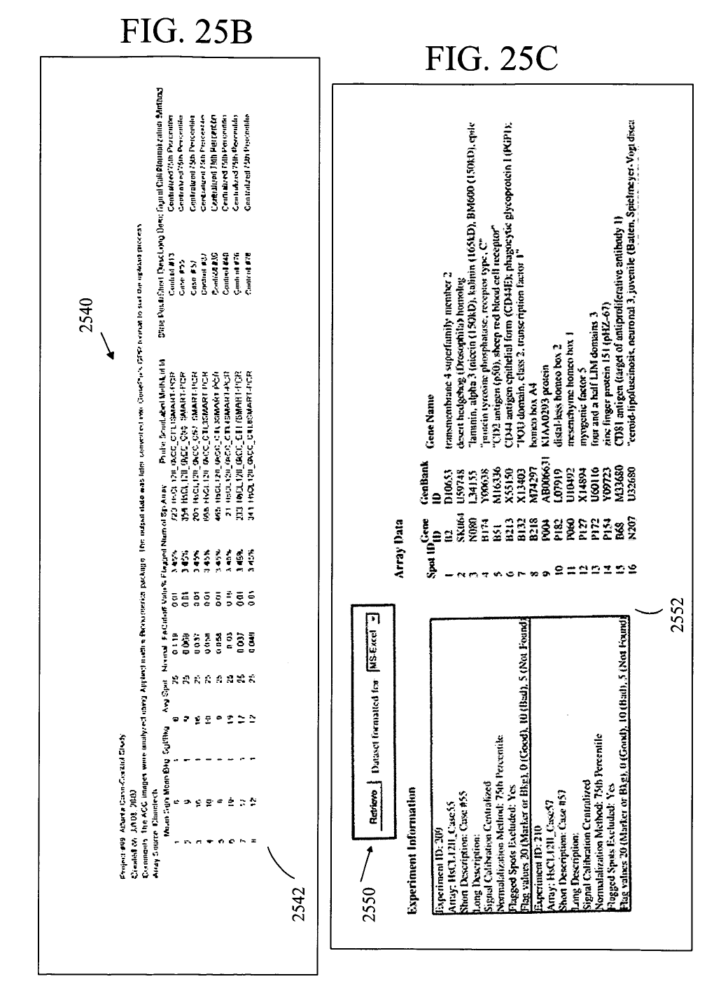

Further analysis can be performed by selecting a tool from the menu 2460, which contains the choices shown in the list box 2220 of FIG. 22. In the example, the user selects “Project Summary Report” and activates the Continue button 2462. As a result, the screen shot 2500 of FIG. 25A is displayed.

FIG. 25A shows a screen shot 2500 of a user interface presenting a summary of information associated with a project and meeting the specified criteria. Each line represents a microarray experiment associated with a subject meeting the specified criteria. Upon activation of the Retrieve button 2510, a report 2542 shown in the screen shot 2540 of D(B) is shown. In the example, the report is exported to MICROSOFT EXCEL spreadsheet format and an EXCEL spreadsheet is shown in the browser window.

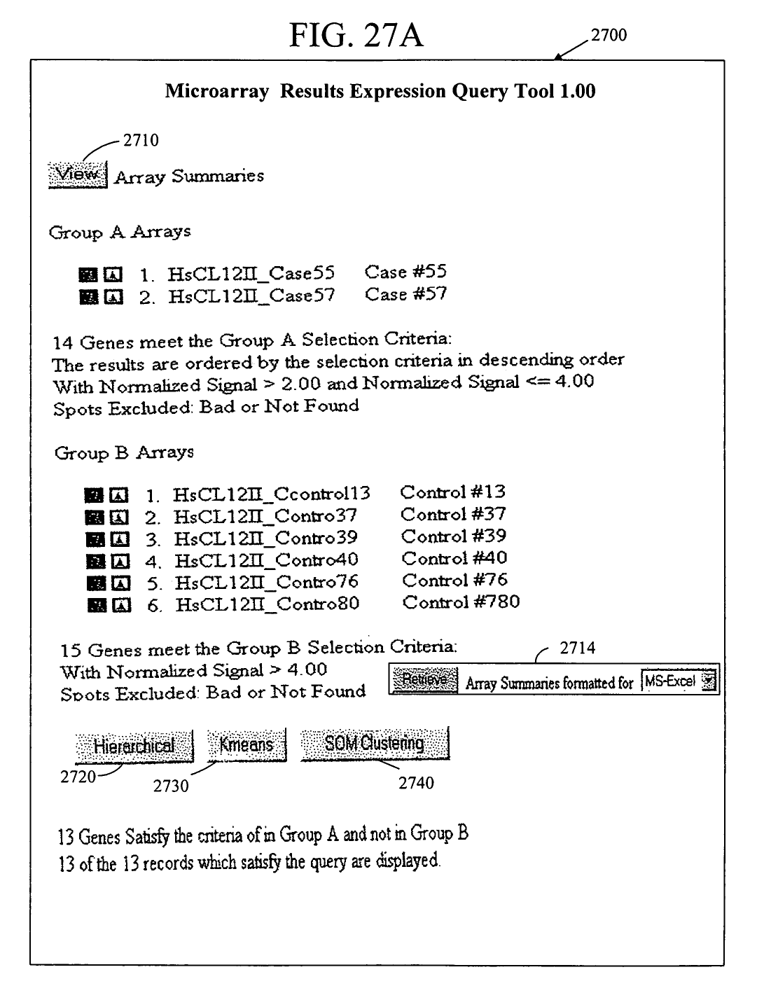

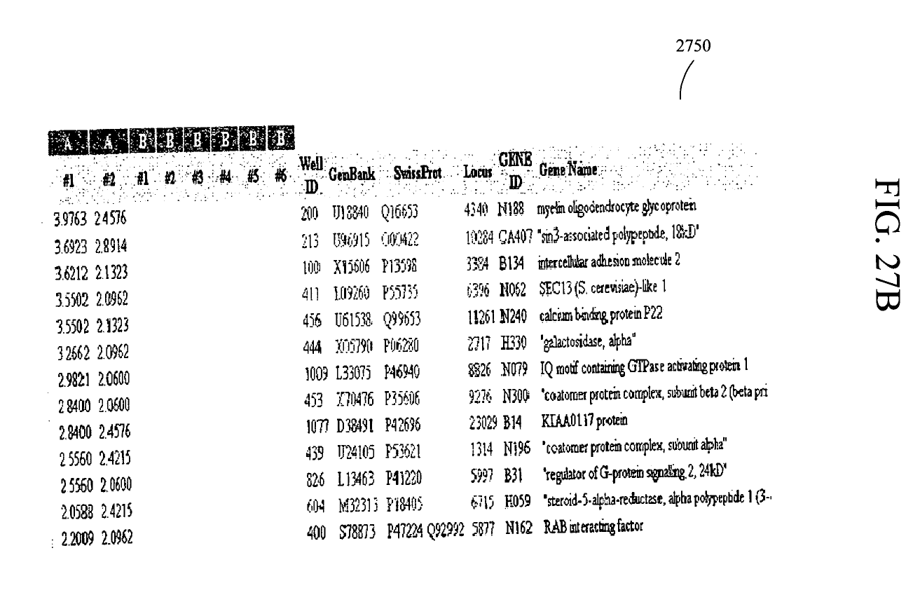

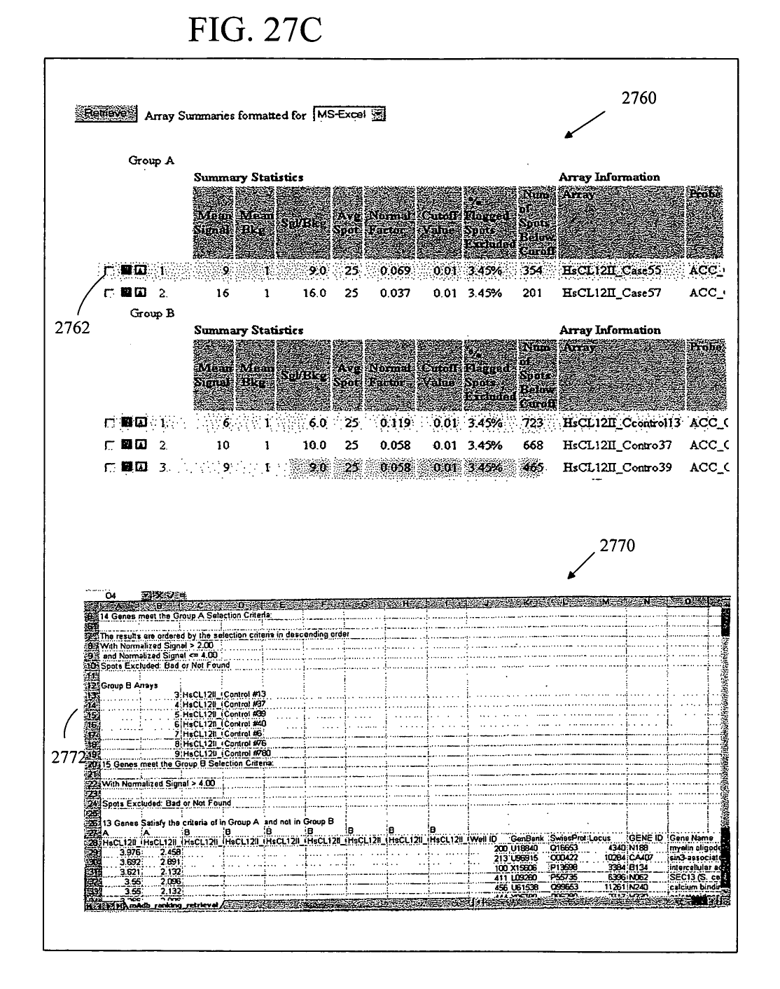

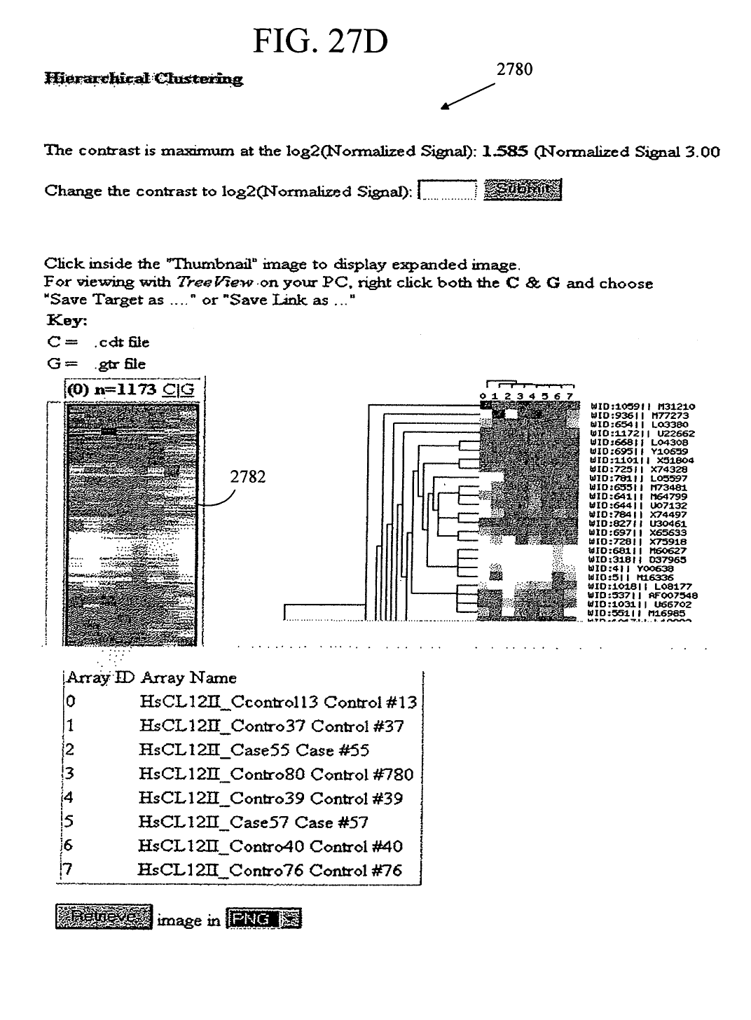

By selecting one or more of the arrays (e.g., via the checkbox 2530) and activating the View Report button 2520, the report 2552 of the screen shot 2550 of FIG. 27B is displayed. The interface also includes a column for the expression level (e.g., normalized signal) and a flag for the genes (e.g., for each selected experiment), not shown. Each line represents a spot of the microarray experiment (e.g., for a gene). In the example, there were over 1,000 spots. The system can support many more spots if desired.

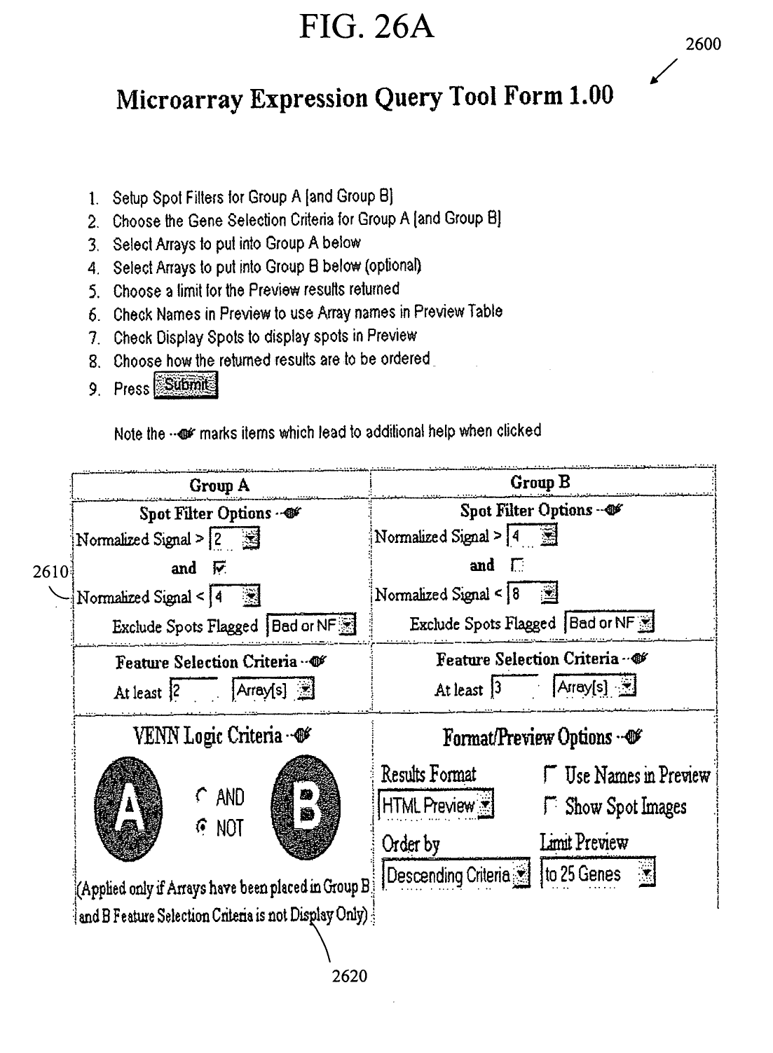

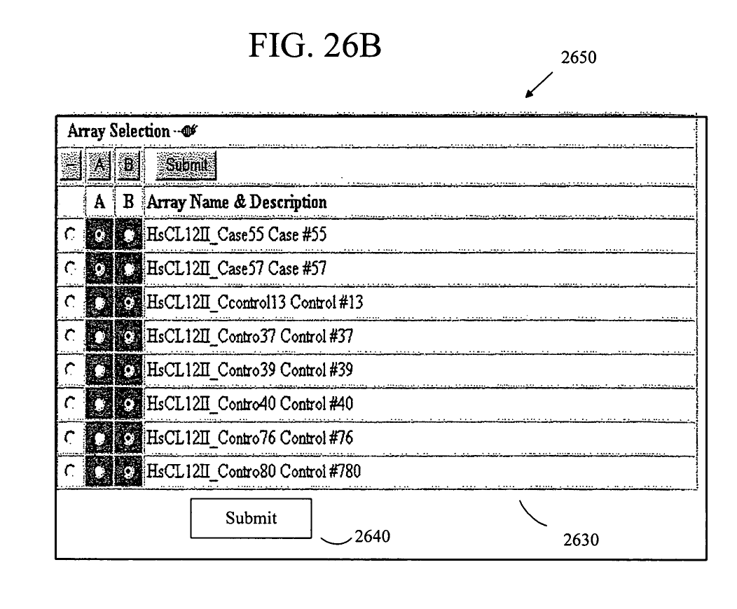

The user can then navigate back to the Epi-Data Search Results window of FIG. 24A and select a different tool from the drop down list box 2460. In the example, the user selects the 1 or 2 Group Logic Retrieval Tool and is presented with the screen shot 2600 of FIGS. 26A and 26B.

FIGS. 26A and 26B show screen shots 2600 and 2650 displaying Microarray Expression Query Tool Forms. A user can specify criteria by which microarray expression is analyzed for the microarrays meeting the earlier-specified criteria (e.g., those specified via the user interface of screen shot 2300).

The user can specify criteria to filter out genes having spots not meeting the criteria (e.g., below a certain level or not found in enough arrays). Genes meeting the criteria are sometimes called “features.” Instead of a number of arrays, a percentage of arrays can be specified in the feature selection criteria.

VENN logic criteria can be specified in the VENN pane 2620. In this way, a user can specify that she is interested in those genes having spots meeting the criteria in group A and group B (or group A but not group B). Arrays can be manually assigned to a different group using the array selection pane 2630. In the example, the cases are in group A, and the controls are in group B.

Upon activation of the submit button 2640, the query is run against the database to produce the results screen shot 2700 of FIGS. 27A, 27B, 27C, and 27D.

FIGS. 27A and 27B show a screen shot 2700 depicting results of the query. The arrays are displayed in their respective groups. The number of genes meeting the criteria are shown for each group, and the VENN logic results are shown (“e.g., 13 Genes Satisfy the criteria of in Group A and not in Group B”). The records 2750 for the genes meeting the criteria are shown. Expression levels and various gene-related data (e.g., gene name) are shown.

Upon activation of the View button 2710, the summary 2762 of screen shot 2760 is shown. Each line represents a microarray experiment. Other columns not appearing in the screen shot include Probe Source, Label Method, Lot Id, Slide Position, Short Description, Long Description, Signal Calibration, and Normalization Method.

Upon activation of the Retrieve button 2714, the summary 2772 shown in the screen shot 2770 of FIG. 27C is shown. In the example, a MICROSOFT EXCEL spreadsheet format has been selected.

Visual analysis of the groups can be performed by selecting clustering options, such as via the Hierarchical button 2720, the Kmeans button 2727, and SOM Clustering button 2740. For example, upon activation of the Hierarchical button 2720, the presentation 2782 in the screen shot 2780 of FIG. 27D is shown. Array IDs are associated with the visualization for the convenience of the viewing user.

When the Kmeans button 2730 is activated, the user can input the following parameters: number of nodes, maximum number of iterations. Also, the following nodes hierarchical clustering options can be specified: genes (e.g., non-centered metric), arrays (e.g., not clustered), and distance metric (e.g., Pearson correlation). Appropriate graphics are then displayed depicting the Kmeans analysis.

Similarly, when the SOM Clustering button 2740 is activated, the user can input the following parameters: X dimension, Y dimension, number of iterations, and whether to initialized with a randomized partition. The same hierarchical clustering options as those for the Kmeans clustering can be specified. Appropriate graphics are then displayed depicting the SOM clustering analysis.

Software to perform the appropriate clustering analysis calculations is widely available (e.g., the Xcluster program developed at Stanford University).

The user can then navigate back to the Epi-Data Search Results window of FIG. 24A and select a different tool from the drop down list box 2460. In the example, the user selects the Scatter Plot Tool and is presented with the screen shot 2800 of FIG. 28.

FIG. 28 shows a screen shot 2800 including a scatter plot 2820 for arrays selected from the boxes 2830 and 2832. The arrays listed in the boxes are those meeting the earlier-specified criteria (e.g., via the screen shot 2300). In the example, the tool supports one array for the x-axis and one array for the y-axis.

When first activated, the information window 2840 displays a summary of the two selected arrays. However, if dots are selected via an elliptically shaped selection area (e.g., via the mouse), information on genes associated with the dots is displayed in the window 2840.

By clicking on the List Visible Points button 2850, a list of the genes associated with the visible dots (e.g., throughout the scatter plot) are shown in the window 2840.

By clicking the Display List button 2850, a list of the genes in the window 2840 are shown in a separate window and can be exported (e.g., to EXCEL spreadsheet format).

By selecting a gene listed in the window 2840, and clicking on the Feature Report button 2880, a report of the gene is shown with information collected from public databases.

The user can then navigate back to the Epi-Data Search Results window of FIG. 24A and select a different tool from the drop down list box 2460. In the example, the user selects the Multi-Array Scatter Plot Tool and is presented with a screen shot similar to that of 2800 of FIG. 28. However, the tool supports one array for the x-axis and one or more arrays for the y-axis. Other functionality is similar to that of the scatter plot tool of FIG. 28.

The user can then navigate back to the Epi-Data Search Results window of FIG. 24A and select a different tool from the drop down list box 2460. In the example, the user selects the Multiple Pair Scatter Plot Tool and is presented with the screen shot 2900 of FIG. 29.

The user can select a pair of arrays via the boxes 2930 and 2932. Upon activation of the button 2940, data for the pair is added to the plot. Other functionality is similar to that of the scatter plot tool of FIG. 28.

The user can then navigate back to the Epi-Data Search Results window of FIG. 24A and select a different tool from the drop down list box 2460. In the example, the user selects “M v A Plot” and is presented with the screen shot 3000 of FIG I.

FIG. 30 shows a screen shot 3000 including an M v. A plot 3020 for arrays selected from the boxes 3030 and 3032. The arrays listed in the boxes are those meeting the earlier-specified criteria (e.g., via the screen shot 2300). In the example, the tool supports one array for the x-axis and one array for the y-axis. Other functionality is similar to that of the scatter plot tool of FIG. 28.

Various other screen shots show additional functionality. For example, the screen shot 3100 of FIG. 31 shows a diagonal selection area 3120 (e.g., at a 45 degree angle), by which a user can easily select outlier dots (e.g., genes).

FIG. 32 shows a screen shot 3200 by which a user can enter criteria for spots (e.g., associated with gene expression levels), including a criterion “PID like” a text string (e.g., “oncogene” or “receptor”) via the pane 3210. Such an interface is useful for scenarios not involving grouped data (e.g., a single group).

Upon activation of the submit button 3280, the results are shown in the screen shot 3300 of FIG. 33.

FIG. 34 shows a screen shot 3400 by which a user can specify subjects by ID. Upon activation of the submit button 3410, the results are shown in the screen shot 3500 of FIG. 35. Each line represents a microarray experiment associated with a specified subject. Analyses can then be run on the selected experiments via selecting a tool from the tools menu 3510 (e.g., listing the analysis tools 2220 shown in FIG. 22).

### EXAMPLE 34

### Exemplary User Manual for Exemplary Implementation of Single Intensity Data

An exemplary user manual for exemplary implementations of the described technologies follows. The user manual describes additional features and characteristics of an exemplary implementation. For example, any of the tools described in the user manual can be used in any of the examples described herein.

### Centers for Disease Control and Prevention Microarray Database (CDC-MADB) System Single Intensity

### User Manual

What's New in CDC-MADB Version 2

This section highlights several key updates to this guide. A more complete description of these enhancements can be found in their respective sections of this user guide.

| Updated Section | Description of update |
|---|---|
| Visualization tools | Java Single and Multi Experiment Array Viewers |
| | and M vs. A Plot. Each tool can be accessed from |
| | the Analysis drop-down list. |
| Create New Project | Added the Array Source and Array Print Set fields. |
| screen | |
| Add New Array | The Array Source and Array Print Set fields are |
| Experiment screen | now automatically populated. |
| | Added two new fields: Signal Calculation and |
| | Normalization Methods. |
| Histogram screen | Screen information has changed. |
| | Added the Retrieve button. |
| | Added Select Bin drop-down list. |
| Project Summary | Screen has been updated with new columns of data, |
| Report | header information and help. |
| Scatter Plot screen | Added new grid lines. |
| | Added new options to the Ratio to Use field. |
| | Added the Lin's Concordance Corr field. |
| | Added Outlier Selection field. |
| | Added List Visible Points button. |
| | The click and drag option on the Scatter Plot grid |
| | has two new columns of data that appear. |
| | The numbers on the X and Y axis change when the |
| | Ratio to Use option is selected. |

Introduction to Centers for Disease Control Microarray Database (CDC-MADB)

Welcome to the Centers for Disease Control and Prevention Microarray Database (CDC-MADB) system, accessible from https://gabs.sra.com/index1.html, and providing the bioinformatics and analysis tools necessary for processing and interpreting gene expression data. The system is designed to fulfill two major roles.

First, CDC-MADB provides a secure data management system for gathering, storing, and managing your experimental information and array data.

Second, CDC-MADB integrates a variety of web accessible tools to support the multiple analytical approaches needed to decipher array data in a more meaningful way.

Getting Started with the CDC-MADB System

Read Chapter 1 “Before Using the CDC-MADB System” to ensure system compatibility. Then turn to Chapter 4 “Upload and Analyze Data” to get an idea of how to interact with the CDC-MADB database. Next, browse through the additional chapters to learn more about the features of the tools provided for analysis of your microarray results.

For questions and additional help, please contact cdcsupport@gabs.sra.com.

Important Points About CDC-MADB

The CDC-MADB has been designed to capture data generated from the software analysis program GenePix, from Axon, Inc (Union City, Calif.).

An interactive web page has been designed to capture three types of information from system users:
1. Project description information
2. Experimental description information
3. Experimental results including the microarray image data and numerical microarray experimental results.
   1. Before Using the CDC-MADB System
   CDC-MADB Compatibility

The CDC-MADB system is designed as a web-based system. The CDC-MADB system is compatible and best performs with:
Internet Browser capability:
   MS Internet Explorer 5 (with Java Virtual Machine Upgrade)
   Netscape 4.0+
Platform capability:
   Windows 95/98/NT (Recommended memory is 256 MB with a minimum of 128 MB)
      About This Manual

This manual assumes that you have basic familiarity with your computer and browser, and therefore does not attempt to explain how to use typical Windows components—dialog boxes, check boxes, list boxes, and drop-down lists. Please refer to your Windows documentation for basic instruction.

For ease of system navigation, this guide uses the following formatting conventions:

| When you see this . . . | It means this . . . |
|---|---|
| [Keystroke] | All keystrokes are denoted |
| | with brackets and in bold type, |
| | (e.g., [Ctrl]). |
| Combination of key | Any string of commands |
| strokes | identifies keystrokes pressed |
| | simultaneously to perform a single operation. |
| [Alt]-[Print Screen] | For example: On a PC, the command |
| | [Alt]-[Print Screen] |
| | means to press and hold the [Alt] key, while |
| | simultaneously pressing the [Print Screen] key. |

Additional help is available online.

2. The CDC-MADB Gateway Homepage

Homepage Access

The CDC-MADB home page is found at https://gabs.sra.com. This home page provides access to a variety of tools (e.g., a gateway link for uploading and analysis tools) and references, which assist in accessing and analyzing gene expression data.

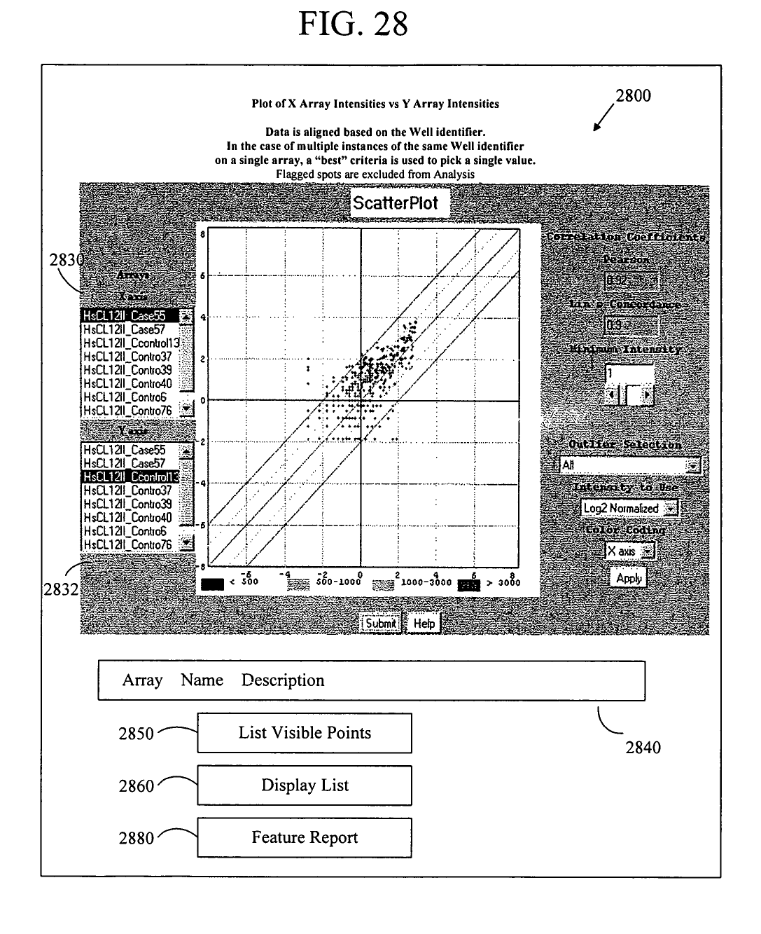

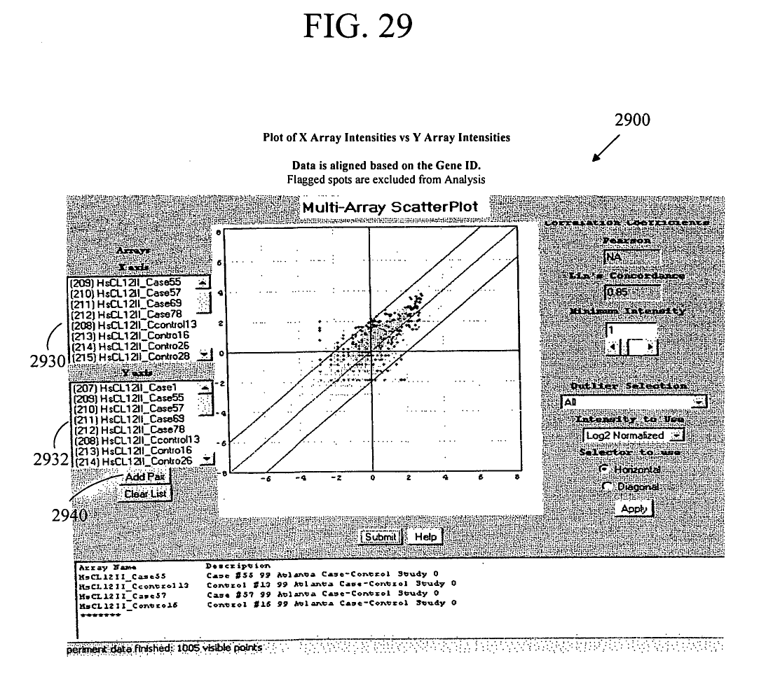

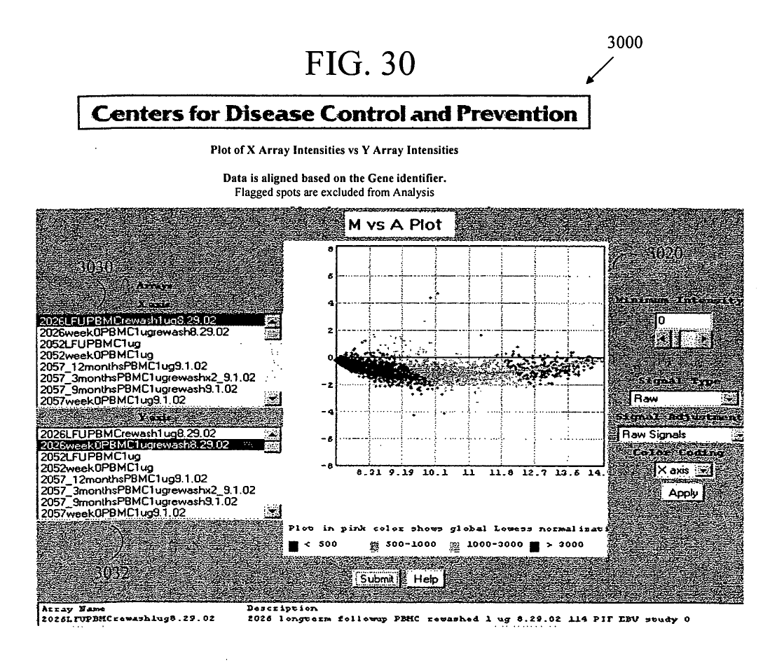

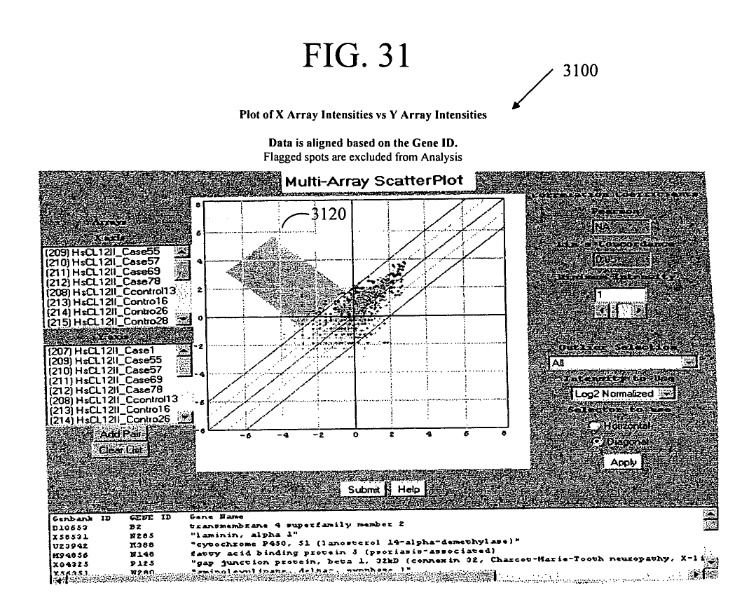

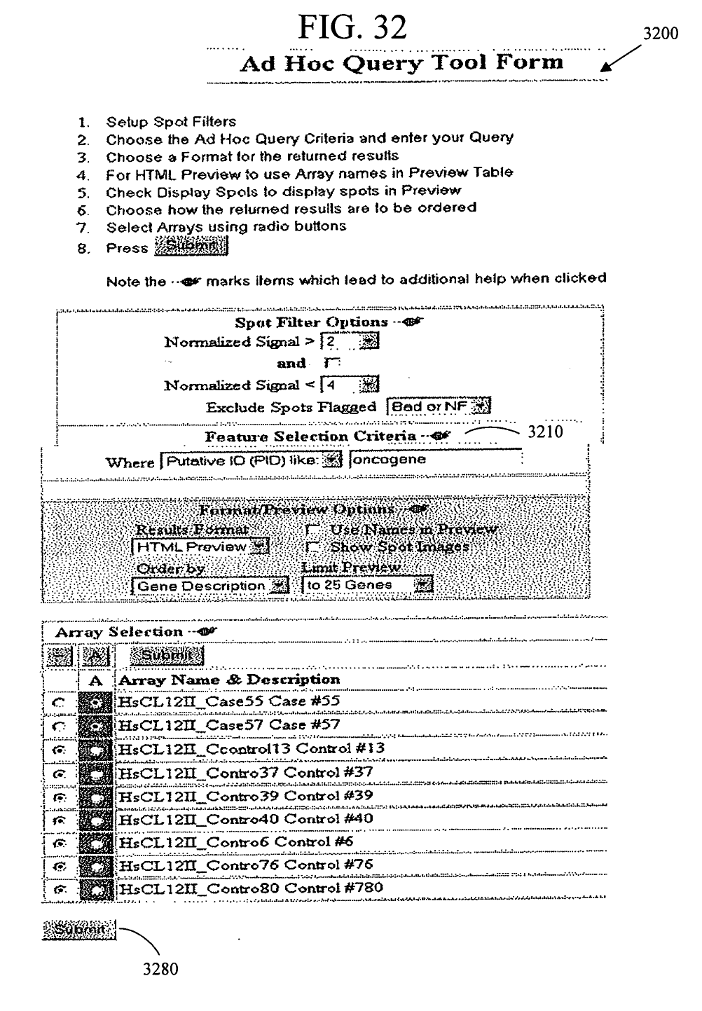

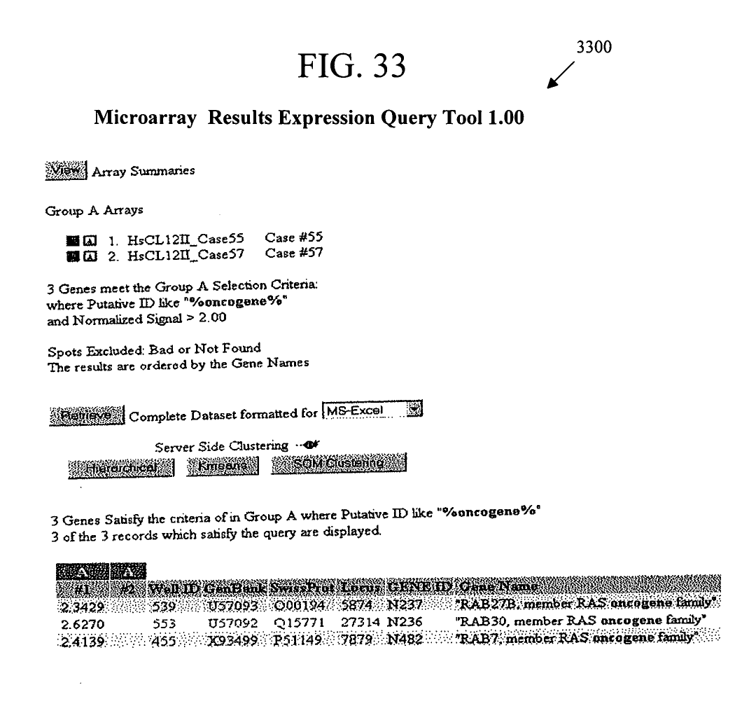

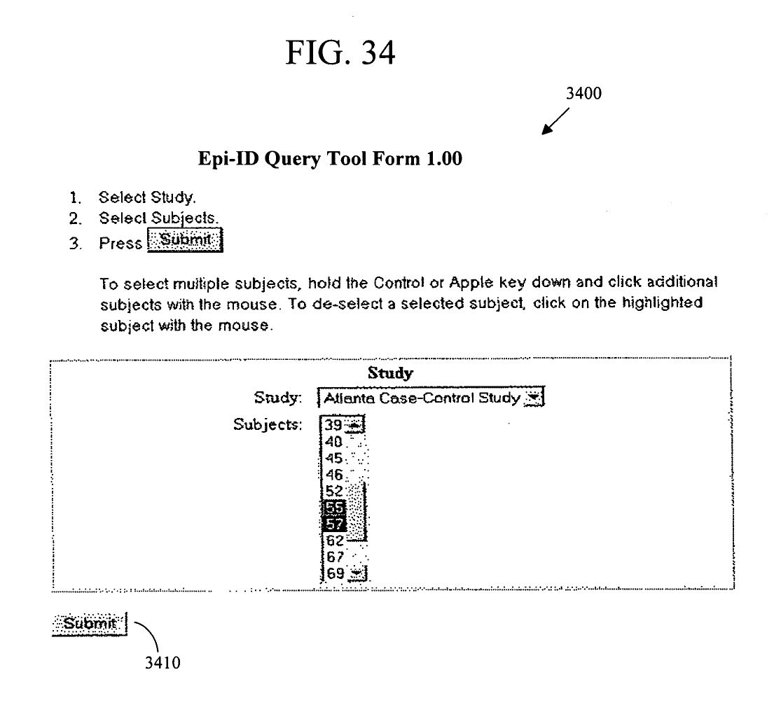

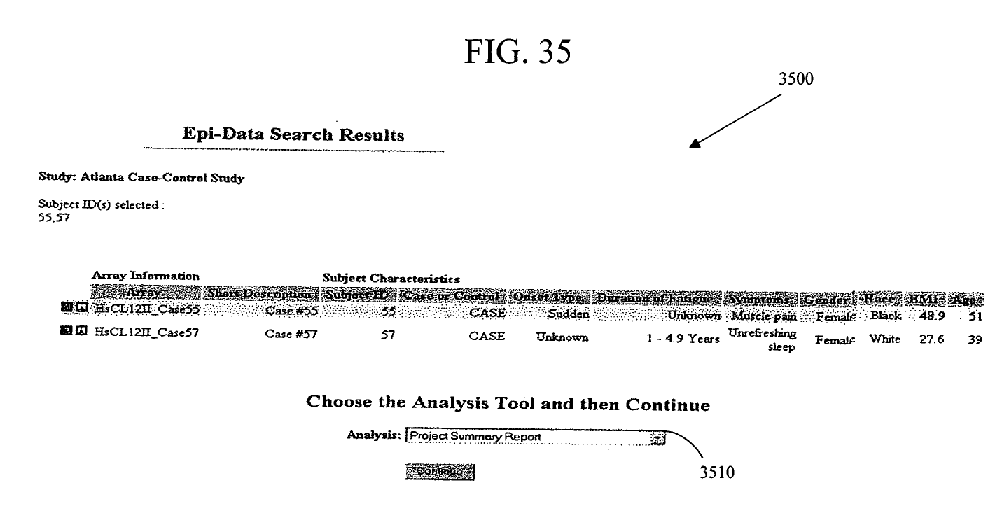

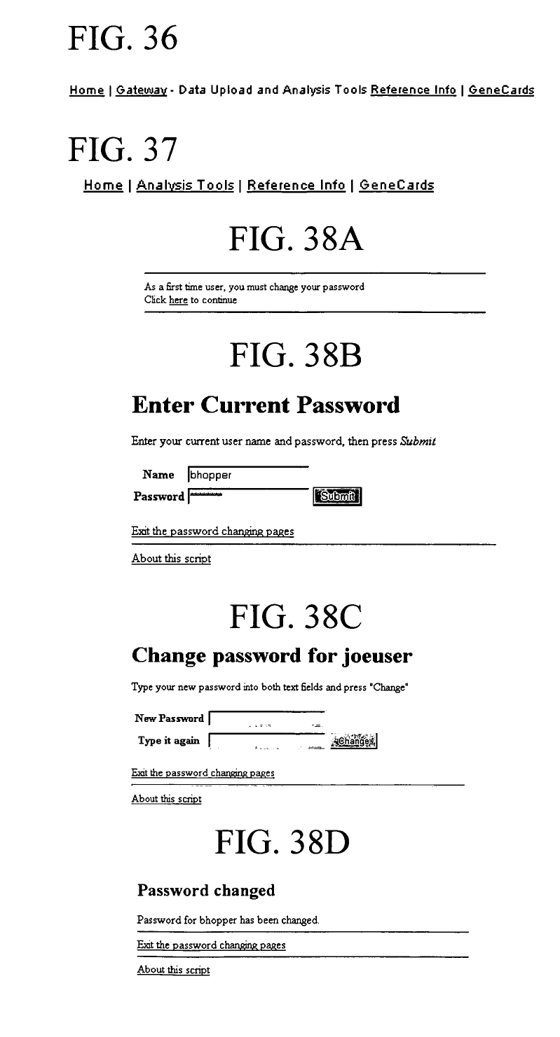

Links at the bottom of the web page can appear as shown in FIG. 36.

When clicked, these links will quickly take you to their respective URLs. Similar links shown in FIG. 37 are found throughout the rest of the system for quick and efficient navigation.

Supporting CDC-MADB Microarray Information

Navigating the CDC-MADB Window

The information found through this web site may be important to your analysis processes. Here is a brief outline of the additional information, resources, and tools available to support the CDC-MADB, which are accessible from the home page.

From the web page, click on the link to retrieve information for further analysis.

Gateway to reach the gateway for Microarray tool analysis.
Note: To access these web pages you must be a registered and have a user login name and password.

Reference Information access to CDC-MADB user manual

Clone Report by Clone, Accession or GID

Tools for mining UniGene Database (local copy of NCBI's UniGene Database)

GeneCards database for Human Genes (CIT mirror of the Weizmann Institute's GeneCards)

MedMiner PubMed mining tool developed by Bioinformatics & Biophysical Pharmacology Group, LMP/NCI

3. User Account Set Up

This chapter instructs you on how to obtain and set up accounts, and provides steps for logging in and changing user privileges for projects.

Obtaining a User Account

Access to CDC-MADB is strictly controlled via the secure socket layer (SSL) protocol and a traditional username and password protocol. SSL security is handled automatically by the CDC-MADB system and it encrypts information traveling between the central server and your workstation. No special software is required to accomplish this high level of security.

An additional level of security is accomplished through controlling access to the system. Each CDC-MADB user is required to have an account on the system. This account allows users to upload experimental data, define projects, view data from other researcher's projects (if permitted), and run the suite of microarray analysis tools.

To obtain a user account, researchers must submit a request, via e-mail, to the CDC-MADB Project Officer, Dr. Suzanne Vernon at sdv2@cdc.gov. Once the request is approved, the CDC-MADB system administrator will create a system account and will forward system login name and password information to the requester via e-mail. Account setup is usually completed within 24 hours of receiving Project Officer approval of the request.

Logging In and Changing Account Information

From the CDC-MADB screen, select the Gateway link.
Note: To access this web site you must be a registered user and have a user login name and password.

1. Enter your login name (your login is case sensitive).

2. Enter your password (your password is case sensitive).

3. If the user information you entered is correct, the Top Level Analysis Selection screen appears.

Changing Your Gateway Password

FIGS. 38A, 38B, 38C, and 38D show screenshots for changing your password.

If this is your first login under this account name, you will be prompted to change your password as shown in FIG. 38A. The request shown in FIG. 38B to re-enter your initial password appears. Type your current password and click Submit. For security purposes, each “*” represents a character of your password.

Next, a screen shown in FIG. 38C to change your password appears. Type your new password into both text fields and click Change.

Unless you made an error typing your new password, an acknowledgement screen shown in 38D appears stating that the change has been made. If your password change was successful, click the Exit the password changing pages link to return to the Top Level Analysis Selection screen.
Note: If an error message appears, enter your password again. Contact your System Administrator if the error persists.

You will be prompted to log in again, using your new password, before the Top Level Analysis Section screen appears.

Logging Out

To ensure that you are logged out of the system, please close your browser window.

Project Access Administration

This option allows you to change the user privileges set for your projects so that others may access them. You are only able to view projects for which you have Administrative Privileges. Granting privileges is divided between single projects and multiple projects.
Note: Be prudent in your privilege granting, especially if you grant Admin privileges to others. Unless you are the project creator, granting Admin privileges to someone else allows him or her to revoke your privileges.
   Changing Privileges for a Single Project

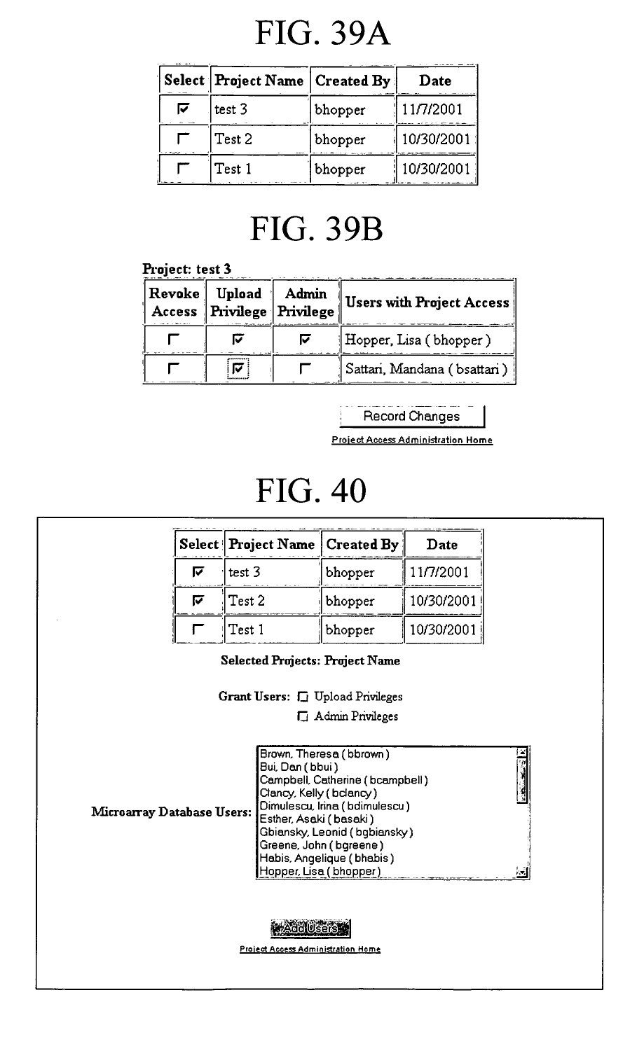

FIGS. 39A and 39B show screenshots for changing privileges for a single project.

1. On the Top Level Analysis Selection screen, select the Project Access Administration link. The Select Project(s) Form web page is displayed in FIG. 39A.

2. Check the box in the Select column that corresponds with the project for which you want to change privileges.

3. To administer user(s) for a single project, click the Single Project button. A Change Privileges Form appears as shown in FIG. 39B.
Note: A message will appear if no project was selected. Click the Back button and try again.

4. The Change Privileges Form allows you to modify the access privileges for users who have already been granted access to the selected project.
Note: If additional users need access, click the Add Users button to select and grant them access as well.

5. Check/uncheck Upload Privilege to grant/revoke rights, respectively, allowing a user to upload arrays to this project.

6. Check/uncheck Admin Privilege to grant/revoke rights, respectively, allowing a user to administer this project.

7. Check Revoke Access to completely revoke a user's access to this project.
Note: A project's creator cannot have access privileges revoked.

8. After making your changes, click Record Changes.

9. A confirmation screen appears stating that the changes are completed.

10. Click Continue on the message screen.

Changing Privileges for Multiple Projects

FIG. 40 shows a screenshot for changing privileges for multiple projects.

1. On the Top Level Analysis Selection screen, select the Project Access Administration link. The Select Project(s) Form web page is displayed in FIG. 40.

2. Check the boxes in the Select column that correspond with the projects for which you want to change privileges.

3. To add user(s) to multiple projects, click Multiple Projects (ADD ONLY).
Note: A message will appear if no project was selected. Click the Back button and try again.

4. Choose which privileges you want to grant (Upload Privileges or Admin Privileges) by checking the box next to it.

5. Scroll through the list and select the CDC-MADB users to whom you want to grant privileges. If you wish to select more than one user, hold down the [Ctrl] key while making your selections.

6. Click Add Users.

7. A confirmation message will appear stating that the changes were made.

8. Click Continue to return to the Project Access Administration page.

Chapter 1. Uploading and Analyzing Data

This chapter describes several activities the user will perform while interacting with the system. These activities include creating and monitoring projects, uploading data to projects, analyzing project data, and obtaining technical support. More detailed information about these analysis tools will be found in later chapters.

Activity: Create a New Project

It is expected that most users of the CDC-MADB system will be performing multiple experiments focused on addressing one or more biological questions. In order to accommodate easy access to experimental information, a logical structure has been adapted to help organize groups of experiments. At this time, it is recommended that a single project should consist of multiple experiments (arrays) that use the same print layout.

At the top level, groups of experiments (arrays) can be referenced as a Project. Multiple experiments will be grouped together within one project. As the number of experiments you submit to the database increases, you will rely on the project groupings to help perform your analysis. Advanced planning is recommended to ensure that logical naming conventions are made regarding organizational information for both your projects and experiments.

The following information will help guide you through creating a new project for your experiments.

Create New Project

On the Top Level Analysis Selection screen, select the Single Intensity Data link under the Links for data uploading header. From the Submit Single Intensity Experiment Data screen, select the Create New Project link. This option allows you to create a new project.

Navigating the Create New Project Window

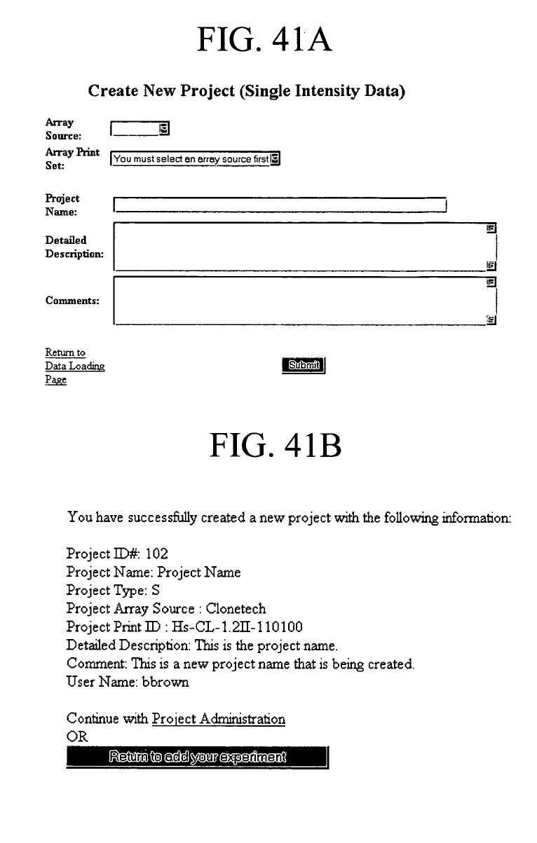

FIGS. 41A and 41B show screenshots creating a new project. The Create New Project window is shown in 41A.

When creating a new project, the user must first select the Array Source and the appropriate Array Print Set from their respective drop-down menus.

Array Source: This drop-down list offers the following sources for selection: Clontech and NCI.

Array Print Set: This is the unique identifier supplied to you from your array manufacturer. This should correspond with an array layout indicating the location and identification of each spot to be analyzed.

Three descriptors are used to identify and distinguish your Project from others. Each is defined below.

1. Project Name: This is a text box, which allows you to create a name for your project. Entry of a project name, with a limit of 128 characters, is required to set up a project.

2. Detailed Description: This text box may be used to describe possible project objectives or provide other clarifying information to others/collaborators who potentially may be sharing your data. This text box is optional.
Note: The maximum field length is 255 characters.

3. Comments: This text box is available to reference or capture any other types of information pertaining to your project. This text box is optional.
Note: The maximum field length is 255 characters.

Once you have completed the fields on this screen, click Submit to proceed.

You will receive a confirmation summarizing your newly created project. The confirmation will appear similar to that of FIG. 41B.

From this page you can proceed to enter your experimental data by clicking on the Return to add your experiment button.

Activity: Upload Experimental Data to the CDC-MADB

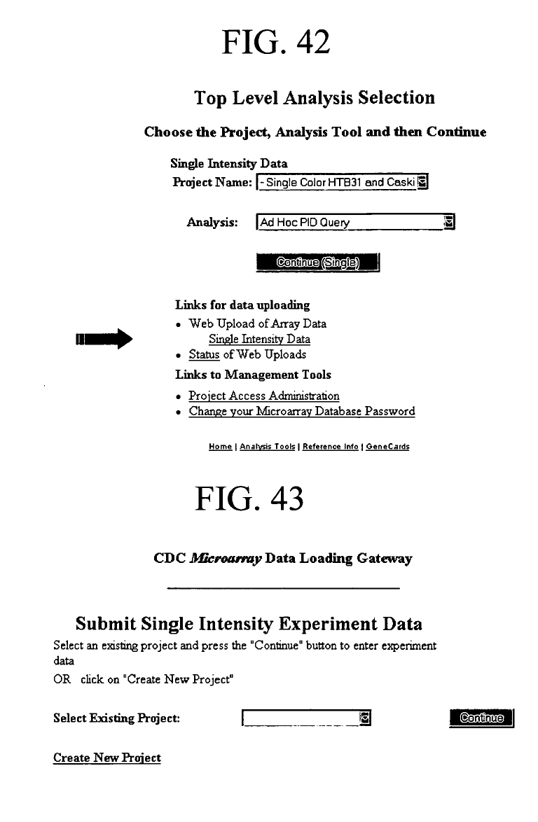

FIG. 42 shows a screenshot for uploading data to the CDC-MADB.

The Upload feature provides the capability to view and analyze a specific data set. At the moment, the link for uploading data is located on the Top Level Analysis Selection tool page.

Under the Links for data uploading heading, click the Single Intensity Data link.

It is possible to be an authorized user on the system and not have been granted upload access, in which case the following message will appear, “You are not authorized to Upload data. Please contact your Systems Administrator.” A hyperlink is provided for convenience.

Submit Experiment Data Window

Navigating the Submit Experiment Data Window

FIG. 43 shows a screenshot for submitting experimental data.

In order to submit experimental data, you must have already created a Project (see the Creating a New Project Activity). Once a Project has been created, one or more experiments with the same print slide layout can be submitted to the project.

To submit experiment data:

1. Select a project from the drop-down list.

2. Click Continue to proceed.

Experiment Information Window

Navigating the Experiment Information Window

When submitting a new experiment to the CDC-MADB database, three types of information will be used to identify and describe your experiment.

1. Experimental description information

2. Image file name

3. Experimental Data file name

Each of these data types will be captured through the web interface. The following are brief descriptions of the fields used to describe your experiment. All fields, except for the Long Description, are required for creating a project.

Array Source: This field will be filled in automatically with information gathered from the Create New Project (Single Intensity Data) screen.

Array Print Set: This field will be filled in automatically with information gathered from the Create New Project (Single Intensity Data) screen.

Array Name: Use this text box to identify an experiment name. It is recommended that you give this some thought if you are expecting to have a number of experiments in your project. A standard naming convention can help you quickly identify your experiments. One such convention is to begin the name of the experiment with part of the Array Print Set Identifier. This text box is limited to 36 characters. An example might be “4 at 6 Hrs.”

Short Description: This text box is limited to 64 characters and is used as a column header to designate your experiment in a multi-experimental analysis tool.

Long Description: Use this text field to describe in more detail experimental information needed for clarification by others/collaborators who potentially may be sharing your data. This text box is limited to 255 characters and is optional.

Probe Source: A name for each labeled probe can be entered in these text boxes. These fields are limited to 64 characters. An example of a probe name might be: “01control” or “ko-3hr.”

Probe Label Method: RT, Double RT, IVT, SMART-PCR, Allyl, or RLS must be selected from the drop-down list to indicate the fluorescent probe label of each probe.

Signal Calculation Method: Select from the following drop-down list options to standardize signal intensities:

Centralized signal: Centralization is the process of moving a distribution so that it is centered over the expected mean. The distribution is centered by shifting the raw signals “(mean foreground−median background)” by minimum such value found in the dataset.
The system computes:
   Raw signal Rsignal=mean foreground−median background
   Shifted signal Ssignal=Rsignal−min(Rsignal)
   Calibrated signal Csignal=100*Ssignal/max(Ssignal)

Note: The above step standardizes the dataset by contracting the statistical distribution so that experimental values can be compared to those with another experiment within the same project.
Normalized signal or Nsignal=Csignal*Normalization Factor Signal that is used everywhere for statistical analysis purpose:
   Signal=Nsignal (if Nsignal>Predetermined Cutoff Value)
   Signal=Cutoff (if Nsignal<=Cutoff Value)
This step ensures that there are no negative or zero values for signal intensity. Since the logarithmic conversion requires the signal value to be greater than zero, the cutoff is usually set to a small positive value (e.g., 0.01).
Signal above background by three SDs: In this approach only the mean signal intensity is used for calibration purpose. However, the cutoff is set differently. The mean and the standard deviation of the “median background” value over an entire array is computed and then any signal intensity below the mean plus three standard deviations of the “median background” is set to zero (0.01).
The system computes:
   Raw signal RSignal=mean foreground Calibrated signal
   Csignal=100*Rsignal/max(Rsignal) Normalized signal
   Nsignal=Csignal*Normalization Factor
Signal that is used everywhere for statistical analysis purpose is determined by setting the cutoff value:
   Cutoff=mean(median background)+3 Standard Deviation
   Signal=Nsignal (if Nsignal>Cutoff)
   Signal=Cutoff (if Nsignal<=Cutoff)
Signal above background by two SDs: This approach is similar to the above one except for the cutoff value. The cutoff is set to the mean plus two standard deviations of the “median background.”
(Signal−Bkg), if Signal above background by three SDs: In this approach the background subtracted mean signal intensity is calibrated. The cutoff is set to the mean plus three standard deviations of the “median background.”
The system computes:
   Raw signal RSignal=mean foreground−median background
   Calibrated signal Csignal=100*Rsignal/max(Rsignal) Normalized signal
   Nsignal=Csignal*Normalization Factor
Signal that is used everywhere for statistical analysis purpose is determined by setting the cutoff value:
   Cutoff=mean(median background)+3 Standard Deviation
   Signal=Nsignal (if mean foreground>Cutoff)
   Signal=Cutoff (if mean foreground<=Cutoff)
(Signal−Bkg), if Signal above background by two SDs: This approach is similar to the above one except for the cutoff value. The cutoff is set to the mean plus two standard deviations of the “median background.”
   Normalization Method: Currently, there are two options available: 50th Percentile (Median) and 75th Percentile. The system sorts the calibrated signals described above, and finds the signal value located at the normalization option selected. The reciprocal of this value is then set as the normalization factor. By doing this we are setting the reference value (median or 75th percentile) of all datasets to one. Be aware that the flagged spots are excluded from all the statistics used in the calibration as well as in the analysis tools.

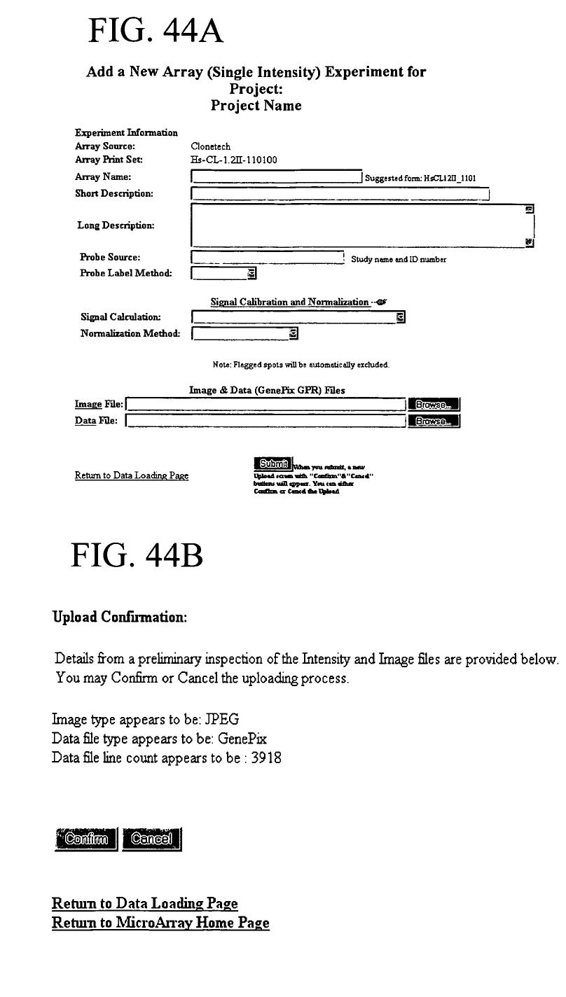

FIG. 44A shows a screenshot for adding a new single intensity array to a project.

Experimental Data Input is captured by interactively uploading file information to the database. To upload your experimental image and data files:

1. Click the Browse button to search for your Experimental Image File on your computer file system.

2. Select the file to upload from the list.

3. Click the Open button. This will automatically indicate the path to your file within the Image File text box.

4. Repeat steps 1-3 to locate your Data File.

5. Click Submit to upload your data.
Note: The Image File and Data File fields must not be empty or you will receive an error message.
Note: The data file is the text file that contains the array data in a tabular format. The image file is the image of the scanned array. The image file must be in JPEG (.jpg) format.

If the system has successfully captured your data, then the screen shown in FIG. 44B will appear.

This confirmation will attempt to:
Evaluate the uploaded files
Determine the image file format (JPEG)
Determine the approximate number of lines in the data file.

To accept this confirmation and continue with the upload process, press the Confirm button. To cancel this upload, press Cancel.

To add an experiment to a different project, click the Return to Data Loading Page link.

To return to the main page, click the Return to MicroArray Home Page link.

Activity: Check the Status of Web Uploads

This page is accessed from the Top Level Analysis Selection screen and provides a status report of successful arrays uploaded by the current user. This page will refresh every ten minutes.

Other Microarray Web Upload reports are available for viewing from this page. These include:
Summary by month of arrays uploaded in the past year
Daily summary of arrays uploaded in the past 90 days
Detailed listing of arrays uploaded within the past 7 days
Detailed listing of all uploaded arrays.
   Activity: View the Project Summary Report

The Project Summary Report is a reporting tool that provides a statistical summary of all experiments in a project, with normalization factor, mean signals, median backgrounds, signal/background ratios, % of features found, and description of the labeled probe.

Selecting a Project Summary Report

A Project to which at least one Experiment has been submitted must be selected before the Project Summary Report tool can be selected.

1. From the CDC-MADB screen, select the Gateway link.
Note: To access this web site you must be a registered user and have a user login name and password.

2. The Top Level Analysis Selection screen is displayed.

3. Select a Project from the Project drop-down list.

4. Select Project Summary Report from the Analysis drop-down list.

5. The Project Summary page is displayed.

Project Summary Report Window

Navigating the Project Summary Report Window

The data results displayed on the Project Summary Report screen can be viewed by three different means. Examples of results are shown below.

1. Array Summaries can be chosen from the drop-down list of array formats and then clicking the Retrieve button. The Project Summary Report captures Array summary formats in MS Excel, PC, Macintosh, and Unix.

2. To view an experiment's image, click the far-left icon on the array summary statistics report.

3. To view the Histogram version, click the Histogram icon on the array summary statistics report.

Results Display

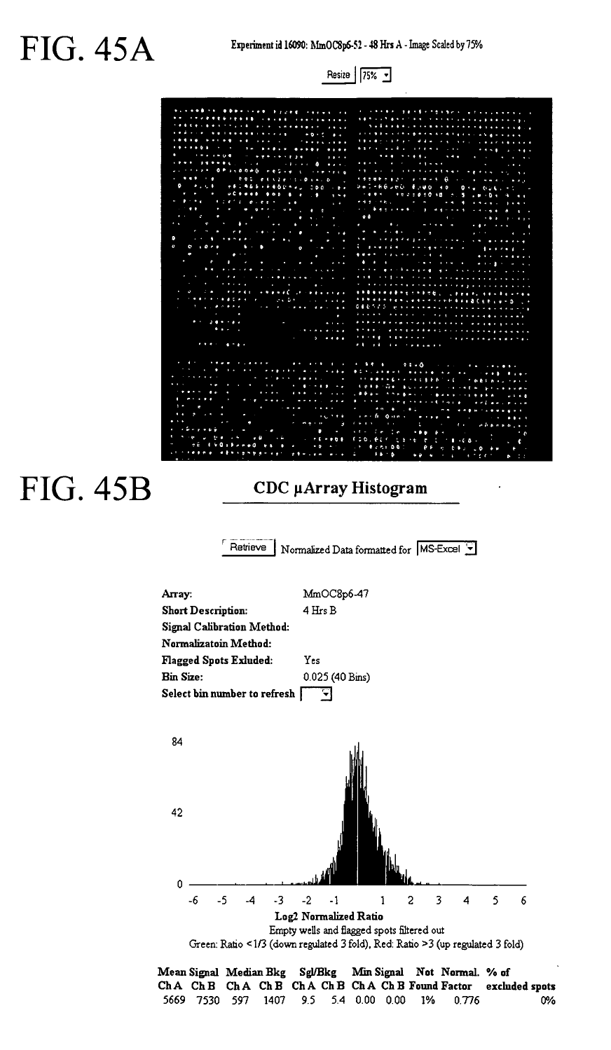

FIGS. 45A and 45B show screen shots of the results.

To change the size of the experiment's image, choose the desired scale from the drop-down list and then press the Resize buttion.

Spot Image

FIG. 45A shows a spot image of the data.

Note: In the system, the spot image can be resized to allow users to view the entire image or zoom into a specific area.

Histogram

If you wish to access this data as a text file, choose the format from the drop-down list, and then press the Retrieve button.

The Histogram shown in FIG. 45B provides a visual chart of the image data.

From the screen you may change the bin size which will refresh the display.

The bin size determines the resolution of the plot. This means that each log unit is divided into a specified number of subunits of intensity values. Once the bin size is determined for each bin location, the number of genes that fit the value is determined and vertical lines are drawn at bin locations depicting the relative count with respect to the max count shown on the Y axis.

Use the drop-down list to select the bin size. The Histogram will be redrawn at the new resolution. The default bin size is 40.

Printing Internet Pages

Many of the File and Edit menu items in Internet Explorer work as they do in other applications.

To print the contents of the current page

1. From the File menu, choose Print, (a dialog box lets you select printing options and begin printing).

2. Or click the Print button in the toolbar (no dialog box will appear—printing will begin automatically).

In Internet Explorer, you can choose Print Preview from the File menu to see a screen display of a printed page.

Activity: Analyze the CDC-MADB Data

Overview of Analysis Tools and Approach

A number of powerful analytical and visualization tools are included in the CDC-MADB system. Detailed descriptions of these tools are provided in the appropriate sections of the manual. A brief summary of these tools is provided here.

1. Scatter Plot Tool: Provides an interactive scatter plot of gene expression intensities for any pair of experiments; allows color-coding of gene intensities and subsetting capabilities.

2. Java Experiment Array Viewer: The Java array viewer is available for both single and multi experiments. These tools were designed to be an intuitive and efficient way to gather significant information from hybridization data.

3. EPI-Data Query: Selects groups of microarray experiments based on demographic and epidemiological information.

4. EPI-ID Query: Selects groups of microarray experiments performed for specific subjects.

5. Ad Hoc PID Query: Provides extensive search and subsetting capabilities. For each array that satisfies a query, the experiment's image and histogram of the gene expression intensities are provided. Genes that satisfy query criteria can be clustered. Hierarchical clustering, Kmeans clustering, or Self-Organizing Maps (SOM) clustering algorithms are available. Results can be either viewed online or retrieved.

6. 1 or 2 Groups Logic Retrieval Tool (VENN Logic): Provides tools to compare two groups of experiments. Query conditions can be set independently for each of the two groups of arrays. Genes selected by the query can be clustered. Hierarchical clustering, Kmeans clustering, and Self-Organizing Maps clustering algorithms are available. Results can be either viewed online or retrieved.
Note: More details about these analysis tools are available in later chapters of the user manual.
   Data Elements for Query

It is assumed that the CDC-MADB system contains data from the microarray experiments (gene expression profiles) and the following (demographic and epidemiological) information for each experiment:
Study
Case/control classification
Age
Gender
BMI (body mass index)
Race
Disease status information (fatigue onset, fatigue duration and observed symptoms)
Date of sample
   Note: The current CDC-MADB system does not provide automated upload capabilities for these data types. This feature is recognized as a part of the systems upgrade requirements.
      Filtering and Retrieving Data Sets

A comparison analysis of the gene expression profiles between healthy subjects and subjects with a disease is the main goal of the CDC-MADB system. To perform this task, subgroups of experiments related to particular groups of subjects are queried from the system. Examples of group definitions are given below:
Subjects from Atlanta Study; 3040 years old; white; males; controls.
Subjects from Atlanta Study; 3040 years old; white; males; with long history of CFS (chronic fatigue syndrome).
Subjects #1, 3, 8 from Atlanta Study

Each query results in a data set that contains gene expression profiles of a particular group of samples. From this sample group, existing CDC-MADB analysis tools can be launched to investigate corresponding microarray results.

Statistical Analysis of Microarray Data

The following approaches to getting started with microarray analysis are suggested. Some of these analytical techniques are currently available in the CDC-MADB system while others may require additional tool sets. Export of data is provided to support these recommendations.

Preprocessing:
Normalization
Imputation of missing values
Subsetting based on percent of missing data or significance of the gene expression difference

Visualization:
Gene expression distributions
Quantile-Quantile plots
Scatter plots

Group Comparison and Discriminant Analysis:
Visual comparisons via scatter plots
Principal component analysis
Multi-Dimensional Scaling
Visual exploratory analysis of correlation matrix
Discriminate analysis
Significance tests (t-test, paired t-test, F-test), validation via permutation tests

Group Discovery and Cluster Analysis:
Hierarchial clustering
Kmeans clustering
SOM clustering

Many of these tools are implemented in the CDC-MADB system. At the later stages, more sophisticated methods can be added. Meanwhile, export capabilities are provided to facilitate data analysis using external software packages.

Chapter 2. Visualization Tools

Introduction

Visualization tools are primarily used to quickly view trends in the data. These trends can be depicted graphically or in more complex images such as dendrogram tree structures or 3-D rotating figures.

Scatter Plot

This applet is a simple visualization and analysis tool for formatting microarray experiment data into a scatter plot. It is designed for analyzing a pair of related experiments. The values used for drawing the plot are the raw (scaled) intensities and the log2 normalized intensities of each clone, assuming that the two experiments have the same number of clones in the same order.

Selecting the Scatter Plot Tool

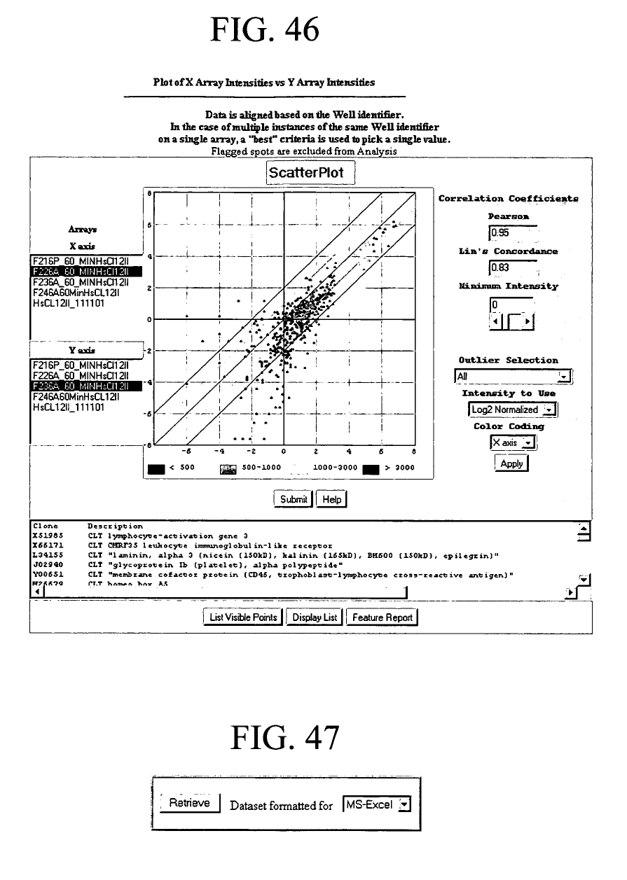

FIG. 46 shows a screenshot of scatter plot tools.

A Project to which at least one Experiment has been submitted must be selected before the Scatter Plot tool can be selected.

1. From the CDC-MADB screen, select the Gateway link.
Note: To access this web site you must be a registered user and have a user login name and password.

2. The Top Level Analysis Selection screen appears.

3. Select a Project from the Project drop-down list.

4. Select Scatter Plot Tool from the Analysis drop-down list.

5. The Scatter Plot Tool screen 4900 is displayed.

Scatter Plot Tool Window

Navigating the Scatter Plot Tool Window

To begin, review and select the Scatter Plot attributes:

1. Experiments: Select experiments from the left of the scatter plot field, labeled “X axis” and “Y axis.” An experiment selected from the “X axis” list will have its data mapped on the horizontal axis, while an experiment selected from the “Y axis” list will be plotted on the vertical axis.

2. Minimum Intensities: These fields are labeled Min Red and Min Green and are found to the right of the scatter plot field and there are two ways to specify the Minimum Intensity: 1) typing the minimum intensity value in the labeled field, or 2) sliding the scroll bar underneath the field to increase or decrease the value. To specify values greater than the maximum values of the scroll bar, type the value directly into the text field. The Minimum Intensity will apply to both experiments. The Mode switch specifies whether the minimum intensities for the red and green channel apply independently or together. “AND” means that a data point has to be above both thresholds in order to be included. “OR” means that a data point will be included if it is above either one of the thresholds. For ordinary use, “AND” should be selected.

3. Intensity To Use: The application can use Log2 Normalized or Raw (Scaled) ratios to draw the scatter plot. The default is Log2 Normalized. The X and Y axis will change depending upon the option selected.

4. Color Coding: To provide a better distinction among the scatter plot data, each data point will be colored based on its intensity values.

5. The Pearson Correlation Coefficient will be calculated each time the Submit button is pressed. Its value is based on the normalized actual data points regardless of whether it is currently being displayed on the scatter plot or not.

6. The Lin's Concordance Correlation will be calculated each time the Submit button is pressed. Its value is based on the normalized actual data points regardless of whether it is currently being displayed on the scatter plot or not.

7. Outlier Selection: These five options: All, Above four fold, Above two fold, Below negative two fold, and Below negative four fold, determine which clones are displayed in the ScatterPlot.

8. The Submit button must be pressed every time you change an experiment so that the data can be updated and redrawn. The first time you click Submit, it may take several minutes to download the experimental data from the database. However, once the experiment data are loaded and you wish to change only the attributes, click the Apply button. This update will be much faster.

Once the data have been plotted, further analysis can be executed with individual or multiple clones. To select clones from the Scatter Plot field, simply click and drag your mouse across the clones in which you are interested. (The screen area will highlight and change color to designate the selected area.) You may select single or multiple clones depending on how many points are within your selection area. Once a clone or a group of clones have been selected:

9. Click the Display List button to view details on the clones within the selection area. (This data will appear in the field below the Scatter Plot as well as in a separate window).

10. Click on a clone in the field below the Scatter Plot and then click on the Feature Report button to retrieve detailed information about that particular clone. When the Feature Report is returned, hyperlinks to related URLs appear in the report. Move your mouse cursor over the report to determine which elements have links. (Usually, these links are noted by colored text.) Click the link for more details.

11. Click the List Visible Points button to view a list of all the clones currently visible on the Scatter Plot. This list appears in the field below the Scatter Plot.

12. The plotted data can also be retrieved in text format. To do this, select the desired format from the drop-down list in the separate window shown in FIG. 47 that was launched when you clicked the Display List button and click the Retrieve button. The data are now displayed as text in the specified format.

Java Single Experiment Array Viewer

The Java Array Viewer is designed to be an intuitive and efficient way to gather significant information from individual hybridization experiments.

Selecting the Java Single Experiment Array Viewer Tool

A project to which at least one experiment has been submitted must be selected before the Java Single Experiment Array Viewer can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select Java Single Experiment Array Viewer, from the Analysis drop-down list.

5. Click Continue.

6. The Single Array Viewer Tool is displayed.

7. Select an Array to view from the drop-down list.

8. Click Continue.

9. The Single Array Viewer Tool histogram is displayed.

Java Single Experiment Array Viewer Window

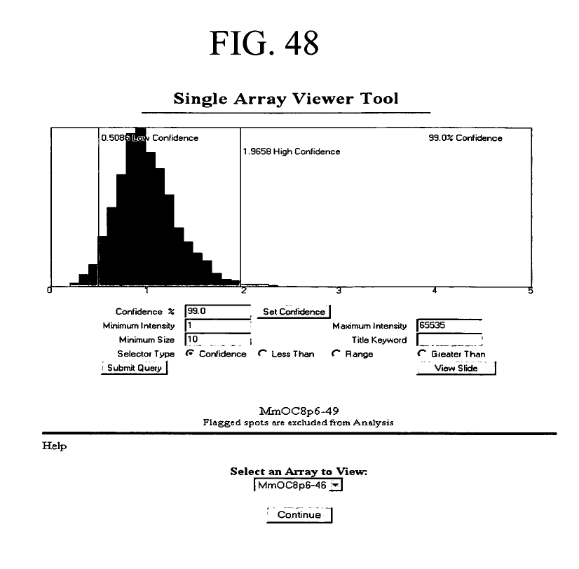

FIG. 48 is a screenshot of the single experiment array viewer tool window.

Navigating the Java Single Experiment Array Viewer Window

The first page of the Array Viewer shows a histogram of the intensity values of the data from one experiment. By default, in the current implementation, flagged spots are excluded. Flagged spots include: Empty, Control, and user flagged problem spots.

To query, review and select the query options:

1. Selector Type: One of four methods can be used to query the data using the histogram: Confidence, Less Than, Range, and Greater Than. Each of these four queries can also be limited by various restrictions. A Minimum Intensity can be set so that only clones that have an intensity above this lower limit are returned. A Maximum Intensity can be set so that the intensity must be below this upper limit. Minimum Size limits clones to those that have a pixel size above a minimum value. Title Keyword restricts the returned clones to only those that have the keyword in their title
Confidence: When this option is chosen, the histogram shows two gray vertical lines that show the upper and lower confidence value for that particular experiment. The initial confidence percentage is set at 99.0%. This value can be edited in the Confidence % field. In order for the new setting to be registered and affect the query, the Set Confidence button must also be clicked.
Range: When this option is chosen, the gray confidence lines are replaced with a pair of blue lines which can be repositioned by clicking the mouse inside the histogram window. The line being repositioned toggles with each mouse click.
Less Than: When this option is chose, the gray confidence lines are replaced with a single blue line, initially positioned at the high confidence mark, which can be repositioned at the high confidence mark, which can be repositioned by clicking the mouse inside the histogram window.
Greater Than: When this option is chosen, the gray confidence lines are replaced with a single blue line, initially positioned at the high confidence mark, which can be repositioned by clicking the mouse inside the histogram window.

2. Submit Query:
Clicking on Submit Query button activates your query. This will automatically return all the clones with an intensity in between those two blue lines positioned on the histogram. When either Greater Than or Less Than is selected, only one line appears for positioning on the histogram. Submit Query returns all the clones Greater Than or Less Than the positioned value. (See below for more information on the Results Window.)

Lastly, on the main page, selecting View Slide will launch the Results Window with no returned clones, but allows you to visually pick a clone on the image and get the hybridization information.

Results

The Results Window is divided into two sections to display the returned clone information. The top window displays a JPEG image of the hybridization. When a clone is returned after a query it is boxed with either a red or green box and a number to reference it to the quantitative data. The lower window shows the quantitative data on each clone. Each row is one particular clone with the following information in each subsequent column. The first column is an index which references the clones to the boxes highlighting the spots in the upper window. The second column shows the internal database clone ID, followed by an Intensity Value, the number of Pixels, and the title.

After a database query, the information is sorted by intensity values from lowest to highest. The lower window is also linked to more information. By clicking on the red counter number, a new window is launched that shows a zoomed in view of the particular clone and repetition of the information. By clicking on the blue clone ID, a comprehensive Feature Report will be displayed in another browser window.

There are several options listed on the bottom of the results window.
Close Frame after new Query: This checkbox is default checked, which means that after a new query on the main page this window will close. If unchecked this window will not close after a new query.
Allow Clone Selection: This checkbox, when selected, will allow you to click on the upper window JPEG and get the hybridization information about particular clones. This is default checked only when you click View Slide; otherwise, it is default unchecked.
Clear List: This button will purge the list of clones returned by a query and/or manually selected.
Display List: This button will result in the list being displayed in a browser window. From there, you can save or print the list. A pathway is not yet fully implemented.
   Java Multi Experiment Array Viewer

The Array Viewer is designed to be an intuitive and efficient way to gather significant information from a series of individual hybridization experiments.

Selecting the Java Multi Experiment Array Viewer Tool

A project to which at least one experiment has been submitted must be selected before the Java Multi Experiment Array Viewer can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select Java Multi Experiment Array Viewer, from the Analysis drop-down list.

5. Click Continue.

6. You will be prompted to log in to the system again.

7. The Multi Array Viewer Tool screen is displayed.

Java Multi Experiment Array Viewer Window

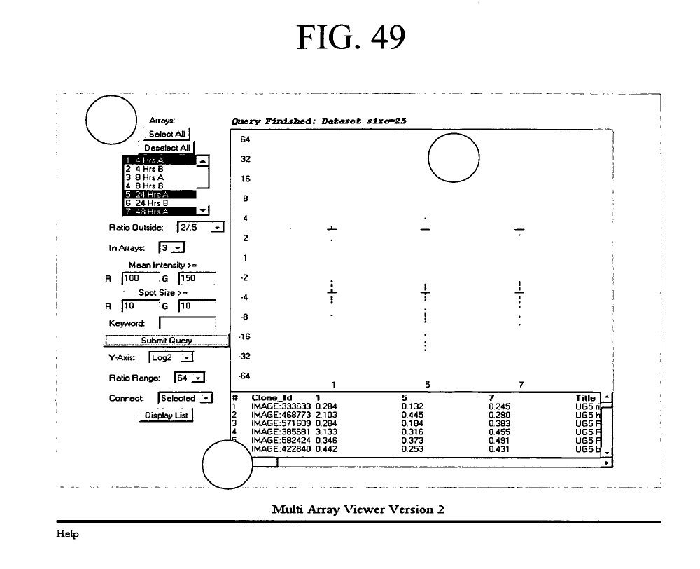

FIG. 49 is a screenshot of the multiple experiment array viewer tool window.

Navigating the Java Multi Experiment Array Viewer Window

The Multi Array Viewer is divided into three sections.

1) The Control panel allows you to select and filter query criteria.

2) The Display panel displays the plot of the experimental data.

3) The Detail panel displays the quantitative information of the clone.

To develop a query, review and select the desired attributes:

1. Select an experiment from the control panel: Intensity Greater Than, In Arrays, Mean Intensity, Spot Size, or Keyword.

2. Once the attributes are set, press the Submit Query button to query the data and determine all the clones that meet the intensity criteria and meet the filter requirements. It will then return the intensities for that clone in all the selected experiments and draw a plot in the Display panel.
Note: Query times average around 10-15 seconds. Please be patient. Also be sure that all selected experiments are from the same print, so that spots across slides correspond.

This display can be displayed in scales. The Y-axis can either be a straight linear progression from 0 to the selected intensity range. (Default is 10). Or the Y-axis can be the log base 2 of the intensities.

In the large display of the clone data, one you can click on a particular spot, and see the intensity of the specified clone across all the selected experiments. An Applet window will be launched that displays additional information about the clone across the selected experiments and also, the quantitative data will be highlighted in the lower display. This can be accomplished also by clicking on the “#” of a clone in the lower display. The Applet window will be launched and the intensity trend will be shown in the large display window.

Lastly, the Clone_id, which appears in the Detail panel, is hyperlinked to the Clone Feature Reports which are linked to other value-added information sources.

Chapter 3. Retrieval and Filtering Tools

Introduction

Retrieval and filtering tools function to bring back specific subsets of data based on the nature of the data. Filtering tools use the characteristics of the data to define a range of interests and retrieval brings back and presents the results. These tools are extremely useful in creating sets of data that contain high value information. Many of these data sets can be saved and imported into supplemental analysis tools.

These are searching tools that query a number of experiments for specific gene information.

Selecting Retrieval or Filtering Tools

A Project to which at least one Experiment has been submitted must be selected before any of the retrieval or filtering tools can be selected.

1. From the CDC-MADB screen, select the Gateway link.
Note: To access this web site you must be a registered user and have a user login name and password.

2. The Top Level Analysis Selection screen is displayed.

3. Select a Project from the Project drop-down list.

4. Choose the desired query tool (EPI-Data Query, EPI-ID Query, Ad Hoc PID Query, or 1 or 2 Groups Logic Retrieval) from the Analysis drop-down list.

5. Click Continue to advance the analysis process.

EPI-Data Query

Overview

EPI-Data is used to select groups of microarray experiments based on demographic and epidemiological information. Data from microarray experiments that satisfy query criteria can be used for analysis with other visualization and query tools.

EPI-Data Query Window

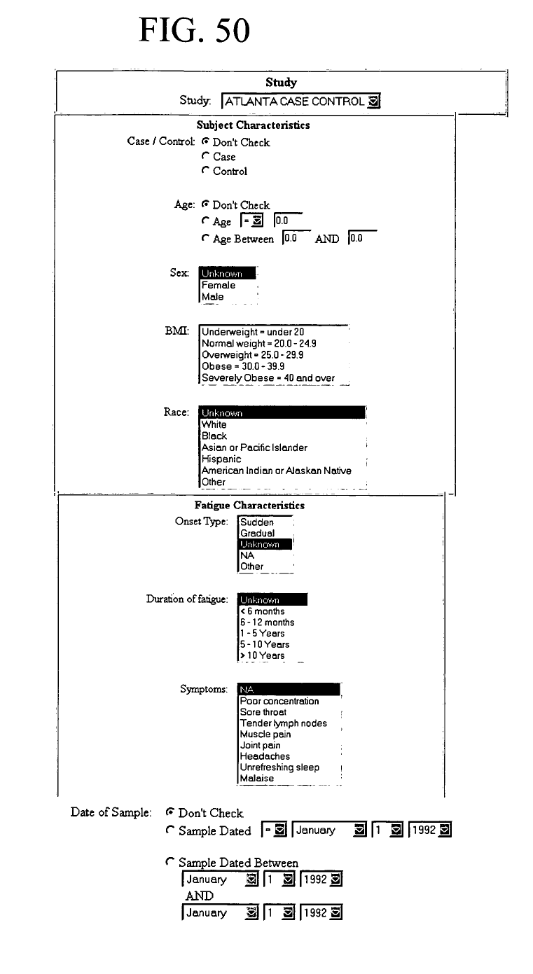

FIG. 50 is a screen shot of the EPI-Data Query Window.

Navigating the EPI-Data Query window

There are four areas on the Epidemiological Data Query Form screen in which data query criteria can be entered. These sections are:
Study
Subject Characteristics
Fatigue Characteristics
Date of Sample

All data fields on the EPI-Data Query Form screen are easy to access through drop-down lists and check boxes.

To begin:

1. Select the Study from the drop-down list.

2. Specify Case/Control. (Optional)

3. Select the criteria for each Subject Characteristic grouping: age, sex, BMI, and race. (Optional)

4. Select the criteria for each Fatigue Characteristic: Onset Type, Duration of fatigue, and Symptoms. (Optional)

5. Select the criteria for the Date of Sample using greater than, less than, or date range values. (Optional)

6. If you prefer not to query on a specific characteristic, then select the Don't Check box.

7. When all options are selected, click Submit to run the query.

Study

Use this drop-down list to choose the study that will filter the Subject and Fatigue Characteristics.

Subject Characteristics

Use these filters to choose subjects that meet specific demographic selection criteria.
Case/Control: Select the Case or Control radio button to set the desired selection criterion. Selecting the Don't Check radio button will deselect this criterion.
Age: These boxes are used to select a specific age or specify minimum and maximum ages for subjects in a group. Selecting the Don't Check radio button will deselect these criteria.
Sex: This pick list is used to select subjects of a specific gender.
BMI: This pick list is used to select subjects with a specific range of Body Mass Index (BMI).
Race: This pick list is used to select subjects with a specific race.
   Fatigue Characteristics

Use these filters to choose subjects that meet specific disease status criteria.
Onset type: This pick list is used to select subjects with specific type of CFS onset.
Duration of fatigue: This pick list is used to select subjects with a specific range of fatigue duration.
Symptoms: This pick list is used to select subjects with specific symptoms. Multiple selections of symptoms are allowed.
   Note: To select multiple items from the Subject or Fatigue Characteristics lists, hold the [Ctrl] key down, while simultaneously clicking on the additional items with the mouse. To de-select an item, click on the highlighted item with the mouse.
      Date of Sample

This group of selections is used to select subjects with a specific sampling date.
Don't check: Selecting the Don't Check radio button will deselect this criterion.
Sample Dated: This series of drop-down lists lets the user select specific dates, using the =, <, or > symbols, corresponding with the month, day, and year drop-down lists.
Sample Dated Between: Selecting this radio button allows the user to specify a date range for his query.
   Submit

When all filters are set to your satisfaction, click the Submit button to activate the tool. For convenience, a Submit button is located at the top and bottom of the Array Selection panel, as well as at the top of the form.

Query Execution

If execution of the query exceeds 20 seconds, an interim page will be displayed indicating that the query is still proceeding. In Internet Explorer, dots will be displayed every few seconds as an indication that the system is still working. When the query is complete, press the Continue button can be pressed to retrieve the results. The results page is held in a temporary file cache and can be bookmarked for later retrieval.

Results

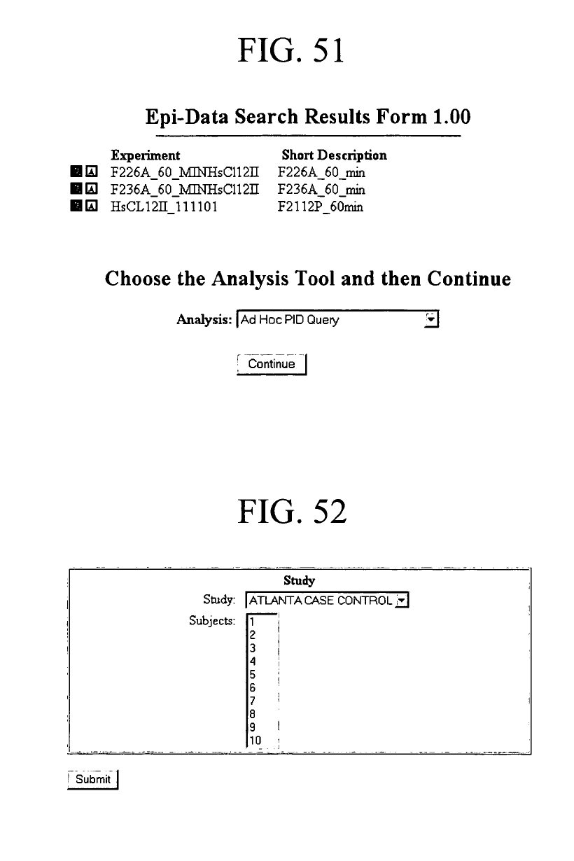

The returned EPI query results are similar to the layout shown in FIG. 51, showing the experiment name and short description. Click on the icons to the left to view either the experiment's image or the histogram version.

If further analysis is warranted, select an analysis tool from the drop-down list to proceed with your examination.

EPI-ID Query

Overview

EPI-ID is a searching tool that queries studies for individual subjects based on demographic and epidemiological information. This tool was designed to help investigators quickly monitor a subject's characteristics and to provide a visual display of the queried information.

EPI-ID Query Window

FIG. 52 shows screen shots for the EPI-ID Query Window 5320.

To review the results of certain subjects, perform the following:

1. Select the Study.

2. Select the Subject(s).

3. Press Submit.
Note: To select multiple Subjects, hold the [Ctrl] key down, while simultaneously clicking on the additional items with the mouse. To de-select an item, click on the highlighted item with the mouse.
   Results

The results of the subjects appear on a new screen shown in FIG. 51. Click on the icons to the left to view either the experiment's image or the Histogram version.

If further analysis is warranted, select an analysis tool from the drop-down list to proceed with your examination.

Ad Hoc PID Query

Overview

The Ad Hoc PID Query is a searching tool that queries a number of experiments for specific gene information. This tool was designed to help investigators quickly monitor genes of interest and to provide a visual display of the queried information.

Ad Hoc PID Query Window

Navigating the Ad Hoc PID Query Window

There are four areas on the Ad Hoc PID Query Tool Form screen in which you can enter data query criteria. An overview of the steps for completing a query appears below, with detailed descriptions of each screen option provided later in this chapter. These are:
Spot Filter Options
Feature Selection Criteria
Format/Preview Options
Array Selection

To begin:
1. Select the desired Signal Intensity/Background.
   Note: Leaving this at the default value of 0.0 will bypass this filter.
2. Select the desired Spot Size and Calibrated Signal.
3. Choose whether to exclude Bad or Bad or NF spots.
4. Choose the Feature Selection Criteria from the drop-down list and enter a relative value in the blank field.
5. Choose the desired Results Format.
6. Check the Use Names in Preview box to display the Array names in the Preview Table.
7. Check the Show Spot Images box to display the spots in the Preview Table.
8. Choose how the returned results are to be ordered with the Order by drop-down menu.
9. Select the desired arrays for query using the radio buttons.
10. When all information is selected, click the Submit button. (The View Array Results section explains how the data are displayed.)
   Spot Filtering

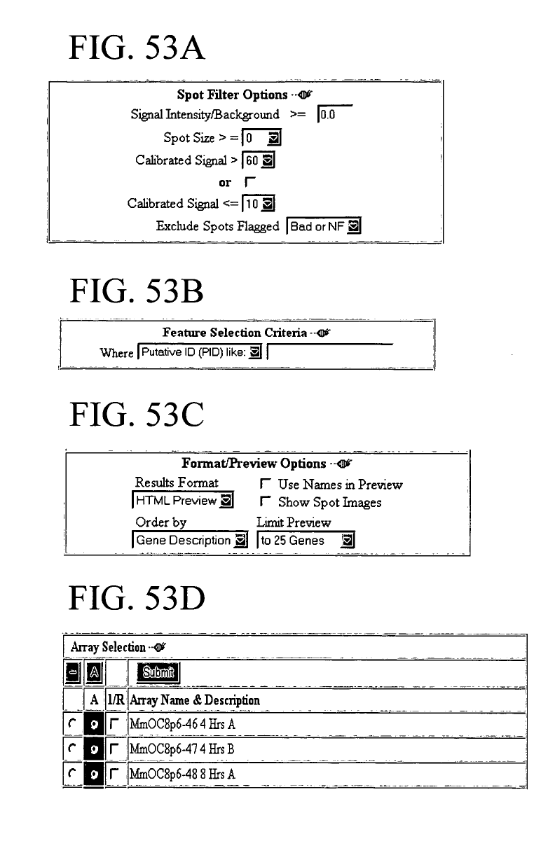

FIG. 53A shows a screenshot of the spot filtering tool of the Ad Hoc PID Query.

Individual array spots can be filtered for spot quality by a number of criteria, to allow those spots greater than or equal to the selected value to pass the filter.
Signal Intensity/Background: This filter simply dictates how strong the signal intensity should be vs. the background intensity for each spot. (Default 0.0)
Spot Size: The percentage of feature pixels with intensities more than one standard deviation above the background pixel intensity at respective wavelength.
Calibrated Signal: This filter sets the minimum absolute intensity of the signal.
Exclude Spots Flagged: A drop-down menu is presented with two options. Bad spots are spots flagged by the user through visual examination of the spot image. NF indicates that the image analysis program does not find the spot.
   Feature Selection Criteria

User can extract array data by searching with one of the following query categories.

FIG. 53B shows a screenshot of the feature selection tool tool of the Ad Hoc PID Query.
Putative ID (PID) like
SwissProt ID is
LocusLink ID is
GenBank ID is
Inventory Well ID is
   Format/Preview Options

These options control the format of the returned results. Use the drop-down lists to view all available options. The data returned is always based on the normalized (calibrated) intensities.

FIG. 53C is a screenshot of the format/preview options tool of the Ad Hoc PID Query.

Results Format: The drop-down menu allows you to choose how you want the results returned and displayed.
HTML Preview: The results are returned in a browser.
Eisen Cluster: The results are returned as a file, formatted for direct input to the Eisen/Stanford Cluster program. It is recommended that you save this as a text or “*.*” file with a “.txt” extension. The data values returned for this format are the Log base 2 of the normalized intensities.
PC, Macintosh and Unix: The results are returned as a TAB delimited text file formatted for the appropriate operating system. The results include a header portion describing the arrays selected and the query.
MS-Excel: The results are returned as MS-Excel content.

Order by: A variety of options can help determine the order in which the data are returned.

Limit Preview: This option limits the number of output rows displayed in the browser, with a default setting of 25 rows. It should be noted that this menu only affects data displayed in the browser; data exported to a tab-delimited file, Eisen Cluster format, or an Excel spreadsheet are always returned in their entirety.

Checkboxes:
Use Names in Preview: Checking the box will display the names of the selected arrays in the browser. If not checked, then the array number keyed in the selected list displayed above the data is used in Preview. It is generally recommended that you leave this box unchecked.
Show Spot Images: Checking the box will display an image of each spot, if available.

CAUTION: This option is highly memory-intensive and is only recommended for checking spot quality when necessary. Checking this box will substantially slow the display of results, particularly on low-bandwidth connections such as those found with a dial-up modem. Each image takes time to be rendered by the web browser.

Array Selection

FIG. 53D is a screenshot of the array selection tool of the Ad Hoc Query.

This section of the Ad Hoc Query tool allows you to select the Arrays to be analyzed.
Selecting Arrays: There are two selection columns to the left of the Array Name & Description list. Initially, the first column (under the “-” button) is selected for all arrays. An Array is de-selected when the radio buttons in this column are marked. To select individual Arrays for analyzing, click the radio button in the “A” column.
Using Button Shortcuts: The “-” and “A” buttons at the top of the column work in the following manner. Clicking on the “-” de-selects all arrays. Clicking on the “A” selects all Arrays. Individual Arrays can still be de-selected by clicking the radio button in the “-” column.
   Note: To function, these buttons require a JavaScript enabled browser.
      Submit

When all filters are set to your satisfaction, click the Submit button to activate the tool. For convenience, a Submit button is located at the top and bottom of the Array Selection panel, as well as at the top of the form.

Query Execution

If execution of the query exceeds 20 seconds, an interim page will be displayed indicating that the query is still proceeding. On Internet Explorer, dots will be displayed every few seconds as an indication that the system is still working. On Internet Explorer, a line will be printed out every two minutes until the query finishes. When the query is complete, press the Continue button can be pressed to retrieve the results. The results page is held in a temporary file cache and can be bookmarked for later retrieval.

Results

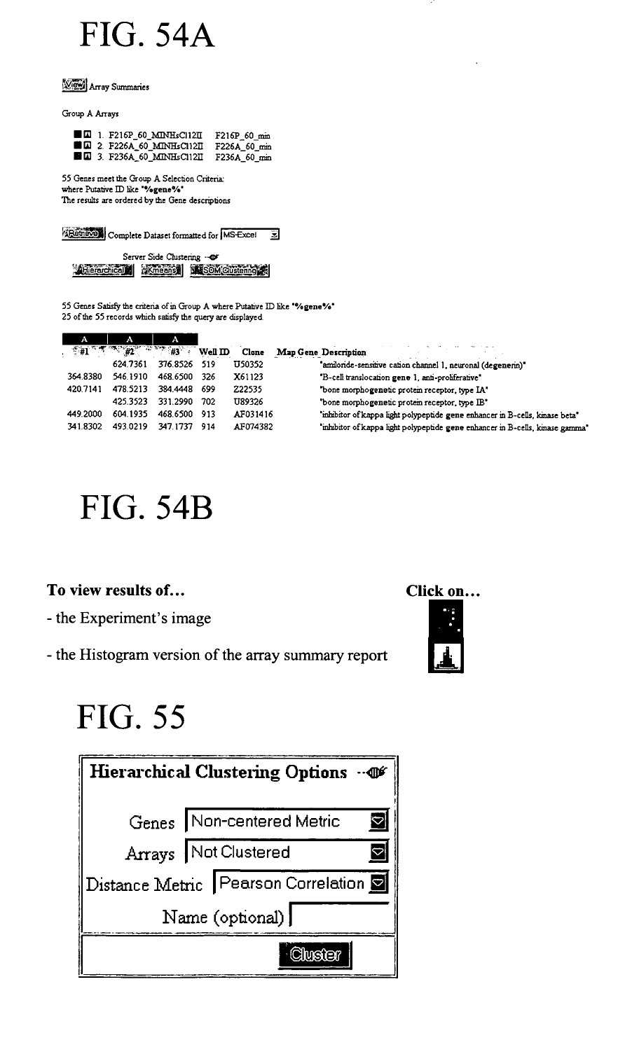

The returned results will be similar to that shown in FIG. 54A, depending on the options you specified on the query selection screen. Place your cursor over any colored text and click to open the link.

Press the View button at the top of the results page to launch the Array Summaries tool in a separate window. Beneath that is a listing of the arrays placed on the form into group A. Below each array listing is a summary of the returned results, indicating how many rows met the specified criteria and repeating the criteria used on the form.

Many URLs related to this query will appear in the returned results. Move your mouse cursor over the screen to determine which elements have links. (Usually, these links are noted by colored text.) Click the link for more details. A Feature Report is displayed.

To the left of each array description are icons to allow viewing the array composite image, or to allow viewing a histogram of the normalized ratios of that array as shown in FIG. 54B. (See the View Project Summary Report activity, for more a more detailed look.)

Server Side Clustering

Clustering is performed using a derivative of the Xcluster program developed at Stanford University by Gavin Sherlock, Head Microarray Informatics.

There are three types of clustering programs available to help you with your analysis: Hierarchical Clustering, Kmeans Clustering, and SOM Clustering. The results displayed will depend on the type of clustering program invoked.

To begin, review and select the clustering steps and options:
1. Select the desired clustering tool.
2. Select the desired options.
3. Click the Cluster button.
4. Your clustered results will be displayed.
   1. Hierarchical Clustering: Specify the Parameters that Control the Hierarchical Clustering.
FIG. 55 is a screenshot of Hierarchical Clustering tool.
Genes & Arrays: The following options can be selected from the associated drop-down lists.
Not Clustered: Choosing this will disable the hierarchical clustering of Genes and/or Arrays.
Non-centered Metric: Uses a non-centered metric.
Median Centered Metric: Uses a centered metric.
Distance Metric: The following options can be selected from the associated drop-down lists.
Pearson Correlation
Euclidean Distance
Name (optional): If you enter a name, it will be used to “tag” your files on the server rather than the server generated tag. This can be handy in managing files you may retrieve with Treeview. The server names will be your MADB login combined with a date/time field.
   2. Kmeans Clustering: Specify Parameters that Control the Partitioning of the Kmeans Clustering.

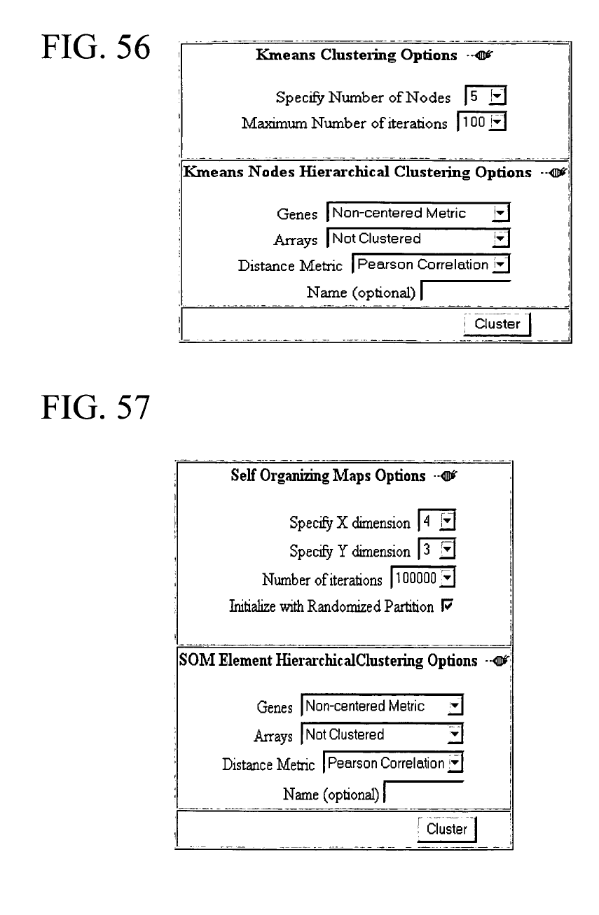

FIG. 56 is a screenshot of the Kmeans Clustering tool.
Specify Number of Nodes: The drop-down list allows you to choose from 2 to 15 Nodes.
Maximum Number of Iterations: The drop-down list allows you to select from a range from 25 to 250 the maximum number iterations. Generally, the Kmeans clustering will converge before the maximum number of iterations is reached.
Kmeans node hierarchical clustering options: The user can specify parameters that control the hierarchical clustering of the individual Kmeans nodes.
Genes & Arrays: The following options can be selected from the associated drop-down lists.
Not Clustered: Choosing this will disable the hierarchical clustering of Genes or Arrays within each Kmeans node.
Non-centered Metric: Uses a non-centered metric.
Median Centered Metric: Uses a centered metric.
Distance Metric: The following options can be selected from the associated drop-down lists.
Pearson Correlation
Euclidean Distance
Name (optional): If you enter a name, it will be used to “tag” your files on the server rather than the server generated tag. This can be handy in managing files you may retrieve with Treeview. The server names will be your MADB login combined with a date/time field.
   3. Self Organizing Maps (SOM) Clustering options: The user can specify parameters which control the partitioning of the 2-dimensional SOM and whether to seed the initial SOM vectors with random numbers. The program currently screens out any Genes whose max(intensity)/min(intensity) across the arrays is <2.

FIG. 57 is a screenshot of the SOM Clustering tool.
X & Y Dimensions: The drop-down lists allow you to choose an X and Y dimension between 1 and 15.
Number of Iterations: The drop-down list allows you to select the number SOM iterations from a range of 50000 to 250000. Each iteration picks a Gene at random and modifies the SOM vector which most closely matches the Gene expression and the neighboring SOM vectors.
Initialize with Randomized Partition: When checked, the initial SOM vectors will be initialized with random numbers.
SOM element hierarchical clustering options: The User can specify parameters that control the hierarchical clustering of the individual SOM elements.
Genes & Arrays: The following options can be selected from the associated drop-down lists.
Not Clustered: Choosing this will disable the hierarchical clustering of Genes or Arrays within each SOM element.
Non-centered Metric: Uses a non-centered metric.
Median Centered Metric: Uses a centered metric.
Distance Metric: The following options can be selected from the associated drop-down lists.
Pearson Correlation
Euclidean Distance
Name (optional): If you enter a name, it will be used to “tag” your files on the server rather than the server generated tag. This can be handy in managing files you may retrieve with Treeview. The server names will be your CDC-MADB login combined with a date/time field.
   Server Side Clustering Results

The data is clustered and the results are returned in a separate window. Click the View Clusters button for a more detailed look at the clustering results. Once the results are displayed, use the features below to guide your interests in seeing the results.

1. To view the text results on your PC, left-click either the C or G character above the image. A separate window appears displaying the data.

2. To save the results on your PC, right-click either the C or G characters above the image, and choose Save Target As from the pop-up menu. Choose the specified path in which to save the file and it will be downloaded.

3. Click on the “Thumbnail” cluster image to display an expanded image view. Once in the expanded view, you may click on the clone line to generate a Clone report, or click on the pattern line to generate a collage of Spot images.

1 or 2 Group Logic Retrieval Tool (VENN Logic)

Overview

The 1 or 2 Group Logic Retrieval Tool is used to compare features on two groups of experiments. It is intended to allow detection of outliers by intensity or average of the intensity across the chosen experiments, as well as finding those rows showing the greatest expression across the arrays. It allows the placing of arrays into one or two groups, and then allowing the feature selection criteria to be set to find arrays that meet those criteria in one group only, or in both groups.

For example, if you had duplicate time points in a project, you could place one replicate into group A and the other into Group B, and ask for those spots that meet the criteria in BOTH of the groups (Boolean AND), or those that met the criteria in Group A only (Boolean NOT). It should be emphasized that this tool can also be used in single group mode by placing all the arrays into Group A.

1 or 2 Group Logic Retrieval Tool Query Window

Navigating the 1 or 2 Group Logic Retrieval Tool Query window

There are five areas on the 1 or 2 Group Logic Retrieval Tool Form in which data query criteria can be entered. An overview of the steps for completing the query appears below with detailed descriptions of each screen option discussed later in this chapter. These sections are:
Spot Filter Options
Feature Selection Criteria
VENN Logic Criteria
Format/Preview Options
   Array Selection

To begin:

1. Select the desired Spot Filters for Group A and B.

2. Choose the Feature Selection Criteria for Group A and B.

3. Select Arrays to put into Group A below.

4. Select Arrays to put into Group B below (optional).

5. Choose a limit for the Preview results that are returned.

6. Check the Use Names in Preview box to display the Array names in the Preview Table.

7. Check the Show Spot Images box to display the spots in Preview 8. Choose how the returned results are to be ordered with the Order by drop-down menu.

9. Click the Submit button.

Spot Filtering

Individual array spots can be filtered for spot quality by a number of criteria, to allow those spots greater than or equal to the selected value to pass the filter.

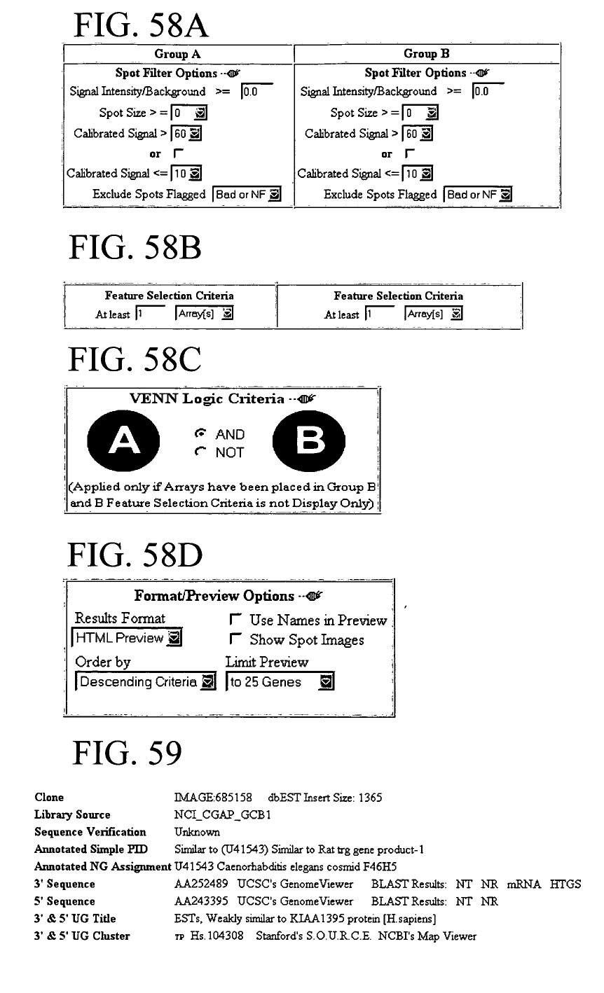

FIG. 58A is a screenshot of the spot filtering tool of the 1 or 2 Group Logic Retrieval Tool Query.
Signal Intensity/Background: This filter simply dictates how strong the signal intensity should be vs. the background intensity for each spot. (Default is 0.0)
Spot Size: The percentage of feature pixels with intensities more than one standard deviation above the background pixel intensity at respective wavelength.
Calibrated Signal: This filter sets the minimum absolute intensity of the signal. If the intensity filter is set for a value of 60, only those array features with a value greater than 60 will pass the filter.
Exclude Spots Flagged: A drop-down menu is presented with two options: Bad spots are spots flagged by the user through visual examination of the spot image. NF indicates that the image analysis program does not find the spot. This filter allows the user to choose to exclude spots flagged as Bad or Not Found (NF) by the image analysis software (the default case), filter only those spots flagged as Bad, or not filter flagged spots at all.
   Feature Selection Criteria

Having filtered the spots for quality, the next panels allow the user to choose outliers exceeding a threshold value in several ways:

FIG. 58B is a screenshot of the feature selection criteria tool of the 1 or 2 Group Logic Retrieval Tool Query.
At Least: The spots on all selected experiments will be evaluated. At Least Spot criteria sets the threshold that in how many experiments (actual number or percentage of the total number of experiments) the gene has to meet the selection criteria.
   VENN Logic Criteria

FIG. 58C is a screenshot of the VENN Logic criteria tool of the 1 or 2 Group Logic Retrieval Tool Query.

This panel allows arrays placed into A and B groups in the Array Selection panel to be compared by Boolean AND or NOT logic. If the AND radio button is selected, only those filtered rows meeting the Feature Selection Criteria in BOTH Groups A and B will be returned. If the NOT radio button is selected, filtered rows meeting the Feature Selection Criteria in Group A but NOT Group B will be returned.

Format/Preview Options

FIG. 58D is a screenshot of the format/preview options tool of the 1 or 2 Group Logic Retrieval Tool Query.

These options allow the user to control the format of the returned results. The data returned are always based on the normalized (calibrated) intensities.

Results Format: This drop-down menu allows you to choose how you want the results returned and displayed.
HTML Preview: The results are returned in a web browser.
Eisen Cluster: The results are returned as a file, formatted for direct input to the Eisen/Stanford Cluster program. It is recommended that you save this as a text or “*.*” file with a “.txt” extension. The data values returned for this format are the LOG base 2 of the normalized intensities.
PC, Macintosh and Unix: The results are returned as a TAB delimited text file formatted for the appropriate operating system. The results include a header portion describing the arrays selected and the query.
MS-Excel: The results are returned as MS-Excel content.

Order by: You may select various options that determine the order in which the data are returned.

Limit Preview: This option limits the number of output rows displayed in the browser, with a default setting of 25 rows. It should be noted that this menu only affects data displayed in the browser; data exported to a tab-delimited file, Eisen Cluster format, or an Excel spreadsheet are always returned in their entirety.

Checkboxes:
Use Names in Preview: Checking this box will display the names of the selected arrays in the web browser. If not checked, then the array number keyed in the selected list displayed above the data is used in Preview. It is generally recommended that you leave this box unchecked.
Show Spot Images: Checking this box will display an image of each spot, if available.

CAUTION: This option is highly memory-intensive and is only recommended for checking spot quality when necessary. Checking this box will substantially slow the display of results, particularly on low-bandwidth connections such as those found with a dial-up modem. Each image takes time to be rendered by the browser.

Array Selection

Arrays can individually be placed into Group A or B by checking the appropriate radio button for each array in the project(s). All arrays can be selected into Group A, or into Group B, by pressing the ‘A’ or ‘B’ button at the top of the A or B columns. All arrays can be deselected by pressing the ‘-’ button in the leftmost column.
Note: To function, these buttons require a JavaScript enabled browser.
   Submit

When all filters are set to your satisfaction, click the Submit button to activate the tool. For convenience, a Submit button is located at the top and bottom of the Array Selection panel, as well as at the top of the form.

Query Execution

If execution of the query exceeds 20 seconds, an interim page will be displayed indicating that the query is still proceeding. On Internet Explorer, dots will be displayed every few seconds as an indication that the system is still working. On Internet Explorer, a line will be printed out every two minutes until the query finishes. When the query is complete, press the Continue button can be pressed to retrieve the results. The results page is held in a temporary file cache and can be bookmarked for later retrieval.

Results

Press the View button at the top of the results page to launch the Array Summaries tool in a separate window. Beneath that is a listing of the arrays placed on the form into group A and into group B (if any). To the left of each array description are icons to allow viewing the array composite image, or to allow viewing a histogram of the normalized ratios of that array. Below each array listing is a summary of the returned results, indicating how many rows met the specified criteria and repeating the criteria used on the form.

Below the individual array listing(s) and individual result summaries is the option to retrieve the complete returned dataset in the format required by the Eisen Cluster program, to retrieve the results as a tab-delimited file for Windows, Macintosh, or UNIX operating systems, or to retrieve the results directly into an Excel spreadsheet.

Next, there is a set of three buttons to choose to cluster this set of rows by hierarchical agglomerative clustering, by Kmeans clustering, or by Self-Organizing Map.

Below the Server-Side Clustering (see the Ad Hoc PID Query section) buttons are the set of results for the Boolean comparison. These indicate how many rows passed the filtering and feature selection criteria for the AND or NOT comparisons of Group A and Group B, if arrays were placed into Group B.
Note: For a more detailed look at the Server-Side Clustering options, see the Ad Hoc Query section of this chapter.

Finally, a table of ratios (and images, if selected) are displayed, with membership in Group A or B denoted at the top of each column. On the right hand side of the table are Well IDs for each feature, which links to a strip image of the row suitable for screen capture for use in a presentation or publication. The clone designation, with links to the feature report; the cytological map location for that gene, if known; the gene symbol, if assigned; and the description of the spot.

Appendix A—Clone Reports

FIG. 59 is a screenshot of a Clone Report. This report has specific clone information that is updated on a regular basis and is linked to a number of peripheral resources such as UniGene and GenBank. In addition, a direct link to the UniGene cluster information is provided, although this information is available in each clone report. The UniGene cluster information is automatically updated weekly to represent the most current information from the UniGene clustering results.

Definitions

Clone—The IMAGE consortium clone used to generate the target spot; hyperlinked to the dbEST record(s) with the IMAGE ID number.
Library Source—library from which the IMAGE clone was derived, taken from the dbEST record.
Sequence Verification—who confirmed the sequence from the IMAGE clone (Stanford, NCI, Unknown).
Annotated Simple PID—short Putative or Probable IDentification of the clone's homology (local annotation).
Annotated NG Assignment—Named Gene assignment which is hyperlinked to the GenBank nucleotide record via the accession number for the Named Gene.
Annotated Categories—Classification of functional role(s) of the Named Gene in the cell.
3′ Sequence—hyperlink to the GenBank record for the 3′ sequence from the IMAGE clone, as well as hyperlinks to the BLASTN and BLASTX output using the 3′ sequence as input.
3′ UG Title—title of the gene (if known) matching the 3′ sequence in the UniGene cluster database.
3′ UG Cluster—link to the UniGene database for the UniGene cluster matching the 3′ sequence.
3′ UG Gene—NCBI LocusLink name for the gene with best homology to the matching UniGene cluster sequence, with links to that gene in the GeneCards database and via Med Miner to the literature on that gene, if available.
3′ UG Cytoband—cytogenetic position of the matching UniGene cluster derived from the UniGene record.
   Appendix B—Data Capture Shortcuts
   PC Shortcuts

[Alt]-[Print Screen] to print a snap shot of a window, place cursor in the window and hold down the [Alt] key and press the [Print Screen] key.

[Ctrl]-[v] to paste the PC window shot into another document, hold down the [Ctrl] key and press the letter [v].

Appendix C—The following references are hereby incorporated by reference herein:

1. Ermolaeva, O., Rastogi, M., Pruitt, K. D., Schuler, G. D., Bittner, M. L., Chen, Y., Simon, R., Meltzer, P., Trent, J. M., and Boguski, M. S. (1998) Nat Genet, 20(1), 19-23.
2. Chen, Y., Dougherty, E. R., and Bittner, M. L. (1997) Journal of Biomedical Optics, 2(4), 364-374.
3. Eisen, M. B., Spellman, P. T., Brown, P. O., and Botstein, D. (1998) Proc Natl Acad Sci USA, 95(25), 14863-8.

### EXAMPLE 35

### Exemplary User Manual for Exemplary Implementation of Dual Probe Data

An exemplary user manual for exemplary implementations of the described technologies follows. The user manual describes additional features and characteristics of an exemplary implementation. For example, any of the tools described in the user manual can be used in any of the examples described herein.

### Centers for Disease Control and Prevention Microarray Database (CDC-MADB) System Dual Probe

### User Manual

What's New in CDC-MADB Version 2

This section highlights several key updates to this guide. A more complete description of these enhancements can be found in their respective sections of this user guide.

| Updated Section | Description of update |
|---|---|
| Visualization tools | Java Single and Multi Experiment Array Viewers |
| | and M vs. A Plot. Each tool |
| | can be accessed from the |
| | Analysis drop-down list. |
| Create New Project | Added the Array Source and Array Print Set fields. |
| screen | |
| Add New Array | The Array Source and Array Print Set fields are now |
| Experiment screen | automatically populated. |
| | Added two new fields: Signal Calculation and |
| | Normalization Methods. |
| Histogram screen | Screen information has changed. |
| | Added the Retrieve button. |
| | Added Select Bin drop-down list. |
| Project Summary | Screen has been updated with new columns of data, |
| Report | header information and help. |
| Scatter Plot screen | Added new grid lines. |
| | Added new options to the Ratio to Use field. |
| | Added the Lin's Concordance Corr field. |
| | Added Outlier Selection field. |
| | Added List Visible Points button. |
| | The click and drag option |
| | on the Scatter Plot grid has |
| | two new columns of data that appear. |
| | The numbers on the X and Y axis change when the |
| | Ratio to Use option is selected. |

Introduction to Centers for Disease Control Microarray Database (CDC-MADB)

Welcome to the Centers for Disease Control and Prevention Microarray Database (CDC-MADB) system, accessible from https://gabs.sra.com/index2.html, and providing the bioinformatics and analysis tools necessary for processing and interpreting gene expression data. The system is designed to fulfill two major roles.

First, CDC-MADB provides a secure data management system for gathering, storing, and managing your experimental information and array data.

Second, CDC-MADB integrates a variety of web accessible tools to support the multiple analytical approaches needed to decipher array data in a more meaningful way.

Getting Started with the CDC-MADB System

Read Chapter 1 “Before Using the CDC-MADB System” to ensure system compatibility. Then turn to Chapter 4 “Upload and Analyze Data” to get an idea of how to interact with the CDC-MADB database. Next, browse through the additional chapters to learn more about the features of the tools provided for analysis of your microarray results.

For questions and additional help, please contact cdcsupport@gabs.sra.com.

Important Points About CDC-MADB

The CDC-MADB has been designed to capture data generated primarily from two different software analysis programs. The first is DeArray (part of Arraysuite) developed by Yidong Chen, NHGRI and the second is GenePix from Axon, Inc (Union City, Calif.).

An interactive web page has been designed to capture three types of information from system users:

1. Project description information

2. Experimental description information

3. Experimental results including the microarray image data and numerical microarray experimental results.

Chapter 1. Before Using the CDC-MADB System

CDC-MADB Compatibility

The CDC-MADB system is designed as a web-based system. The system is compatible and best performed with:
Internet browser capability:
   MS Internet Explorer 5.0+(with Java Virtual Machine Upgrade)
Platform capability:
   Windows 95/98/NT (Recommended memory is 256 MB with a minimum of 128 MB)
      About This Manual

This manual assumes that you have basic familiarity with your computer and browser, and therefore does not attempt to explain how to use typical Windows components-dialog boxes, check boxes, list boxes and drop-down lists. Please refer to your Windows documentation for basic instruction.

For ease of system navigation, this guide uses the following formatting conventions:

| When you see this . . . | It means this . . . |
|---|---|
| [Keystroke] | All keystrokes are denoted with |
| | brackets, (e.g., [Ctrl]). |
| Combination of key | Any string of commands |
| strokes | identifies keystrokes pressed |
| | simultaneously to perform a single operation. |
| [Alt]-[Print Screen] | For example: On a PC, the command [Alt]-[Print |
| | Screen] means to press and |
| | hold the [Alt] key, while |
| | simultaneously pressing the [Print Screen] key. |

Additional help is available online by clicking on the bee icon.

Chapter 2 The CDC-MADB Gateway Homepage

Homepage Access

The CDC-MADB home page, https://gabs.sra.com/index2.html, can be accessed through this link. This home page provides access to a variety of tools (e.g., a gateway link for uploading and analysis tools) and references, which assist in accessing and analyzing gene expression data.

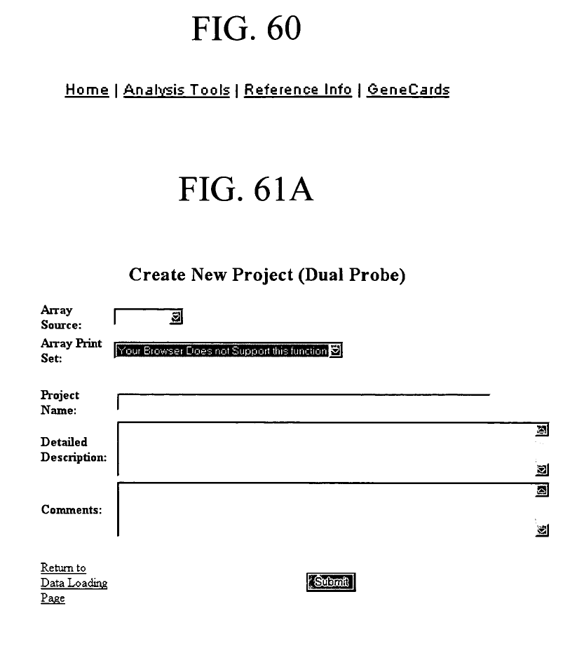

Links can appear at the bottom of the web page as shown in FIG. 60.

When clicked, these links will quickly take you to their respective URLs.

These are found throughout the system for quick and efficient navigation.

Supporting CDC-MADB Microarray Information

Navigating the CDC-MADB Window

The information found through this web site may be important to your analysis processes. Here is a brief outline of the additional information, resources, and tools available to support the CDC-MADB, which are accessible from the home page.

From the web page, click on the link to retrieve relative information for further analysis.

Gateway to reach the gateway for Microarray tool analysis.
Note: To access these web pages you must be a registered user and have a user login and password.

Reference Information access to CDC-MADB user manual

Clone Report by Clone, Accession, or GID

ChipSearch Text based search of Hs Oncochip Set using GeneCard Search Engine

Tools for mining UniGene Database (local copy of NCBI's UniGene Database)

GeneCards database for Human Genes (CIT mirror of the Weizmann Institute's GeneCards)

MedMiner: PubMed mining tool developed by Bioinformatics & Biophysical Pharmacology Group, LMP/NCI

Chapter 3. User Account Set Up

This chapter instructs you on how to obtain and set up user accounts, and provides steps for logging in and changing user privileges for projects.

Step 1. Obtaining a User Account

Access to CDC-MADB is strictly controlled via the secure socket layer (SSL) protocol and a traditional username and password protocol. SSL security is handled automatically by the CDC-MADB system and it encrypts information traveling between the central server and your workstation. No special software is required to accomplish this high level of security.

An additional level of security is accomplished through controlling access to the system. Each CDC-MADB user is required to have an account on the system. This account allows you to upload experimental data, define projects, view data from other researcher's projects (if permitted), and run the suite of microarray analysis tools.

To obtain a user account, researchers must submit a request, via e-mail, to the CDC-MADB Project Officer, Dr. Suzanne Vernon at sdv2@cdc.gov. Once the request is approved, the CDC-MADB system administrator will create a system account and will forward system login name and password information to the requester via e-mail. Account setup is usually completed within 24 hours of receiving Project Officer approval of the request.

Logging In and Changing Account Information

From the CDC-MADB screen, select Gateway.
Note: To access this web site you must be a registered user and have a user login name and password.

4. Enter your login name (your login name is case sensitive)

5. Enter your password (your password is case sensitive).

6. If the user information you entered is correct, the Top Level Analysis Selection screen appears.

Changing Your Gateway Password

If this is your first login with this account name, you will be prompted to change your password as shown in the screenshot in FIG. 38A.

A request to re-enter your initial password appears in FIG. 38B. Type your current password and click Submit. For security purposes, each “*” represents a character of your password.

Next, a screen to change your password appears as shown in FIG. 38C. Type your new password into both text fields and click Change.

Unless you made an error typing your new password, an acknowledgement screen as shown in FIG. 38D appears stating that the change has been made. If your password change was successful, click the Exit the password changing pages link to return to the main page.
Note: If an error message appears, enter your password again. Contact your System Administrator if the error persists.

You will be prompted to log in again, using your new password, before the Top Level Analysis Section screen appears.

Logging Out

Please close your browser window to log out of the CDC-MADB system.

Project Access Administration

This option allows the privileges for your projects to be changed. Changes include granting permission so that others may access your projects. You are only able to view projects for which you have Administrative Privileges. Granting privileges is divided between single projects and multiple projects.
Note: Be prudent in your privilege granting, especially if you grant Admin privileges to others. Unless you are the project creator, granting Admin privileges to someone else allows him or her to revoke your privileges.
   Changing Privileges for a Single Project

1. From the Top Level Analysis Selection screen, click the Project Access Administration link. The Select Project(s) Form web page is displayed in FIG. 39A.

2. Check the box in the Select column that corresponds with the project for which you want to change privileges.

3. To administer user(s) for a single project, click the Single Project button. A Change Privileges Form appears as shown in FIG. 39B.
Note: A message will appear if no project was selected. Click the Back button and try again.

4. The Change Privileges Form allows you to modify the access privileges for users who have already been granted access to the selected project.
Note: If additional users need access, click Add Users to grant them access to this project.

5. Check/uncheck Upload Privilege to grant/revoke rights allowing a user to upload arrays to this project.

6. Check/uncheck Admin Privilege to grant/revoke rights allowing a user to administer this project.

7. Check Revoke Access to completely revoke a user's access to this project.
Note: A project's creator cannot have his/her access privileges revoked.

8. After making your changes, click Record Changes.

9. A confirmation screen will appear stating that the changes were completed.

10. Click Continue to return to the Project Access Administration page.

Changing Privileges for Multiple Projects

FIG. 40 shows a screenshot for changing privileges for multiple projects.

1. From the Top Level Analysis Selection screen, click the Project Access Administration link. The Select Project(s) Form screen is displayed.

2. Check the boxes in the Select column that correspond with the projects for which you want to change privileges.

3. To add user(s) to multiple projects, click the Multiple Projects (ADD ONLY) button.
Note: A message will appear if no project was selected. Click the browser's Back button and try again.

4. Choose which privileges you want to grant (Upload Privileges or Admin Privileges) by checking the box next to it.

5. Scroll through the list and select the MADB users to whom you want to grant privileges. If you wish to select more than one user, hold down the [Ctrl] key while making your selections.

6. Click Add Users.

7. A confirmation message will appear stating that the changes were made.

8. Click Continue to return to the Project Access Administration page.

Chapter 4. Uploading and Analyzing Data

This chapter describes several activities the user will perform while interacting with the system. Some of the topics discussed are creating and monitoring projects, uploading data to projects, analyzing project data, and obtaining user support. More detailed information about these analysis tools will be found in later chapters.

Activity: Creating a New Project

It is expected that most users of the CDC-MADB system will be performing multiple experiments focused on addressing one or more biological questions. In order to accommodate easy access to experimental information, a logical structure has been adapted to help organize groups of experiments. At this time, it is recommended that a single project should consist of multiple experiments (arrays) that use the same print layout.

At the top level, groups of experiments (arrays) can be referenced as a Project. Multiple experiments will be grouped together within one project. As the number of experiments you submit to the database increases, you will rely on the project groupings to help perform your analysis. Advanced planning is recommended to ensure that logical naming conventions are made regarding organizational information for both your projects and experiments.

The following information will help guide you through creating a new project for your experiments.

Create New Project

From the Top Level Analysis Selection screen, click the Upload link under the Links for data uploading header. From the Submit Experiment Data screen, click Create New Project. This option allows you to create a new project.

Navigating the Create New Project Window

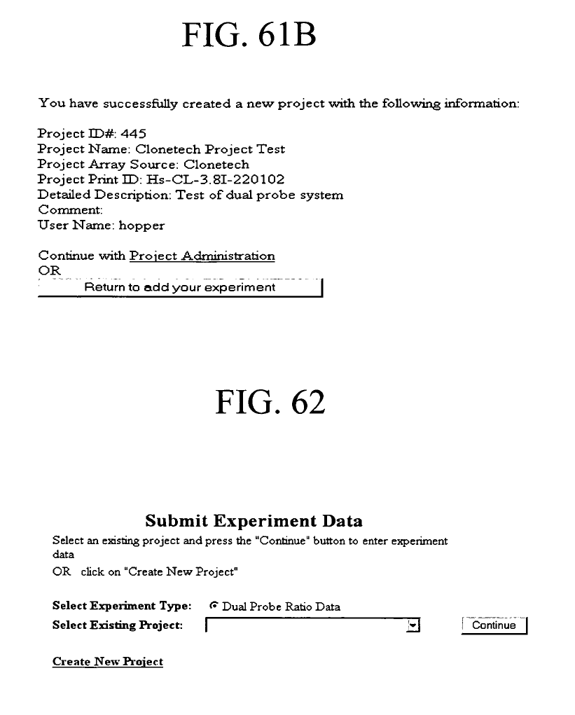

FIG. 61A is a screenshot of the create new project tool for dual probe data.

When creating a new project, the user must first select the Array Source and the appropriate Array Print Set from their respective drop-down menus.

Array Source: Select either Clontech or NCI as the desired source from the drop-down list.

Array Print Set: Select the identifier from the drop-down list. The relative

Array Print Set options will be contingent upon on your Array Source selection.

Three descriptors are used to identify and distinguish your Project from others. Each is defined below.

1. Project Name: This is a text box, which allows you to create a name for your project. Entry of a project name, with a limit of 128 characters, is required to set up a project.

2. Detailed Description: This text box may be used to describe possible project objectives or provide other clarifying information to others/collaborators who potentially may be sharing your data. This field is optional.
Note: The maximum field length is 255 characters.

3. Comments: This text box is available to reference or capture any other types of information pertaining to your project. This field is optional.

Once the fields on this screen have been completed, click Submit to proceed.

You will receive a confirmation summarizing your newly created project as shown in FIG. 61B.

From this page you can proceed to enter your experimental data by clicking on the Return to add your experiment button.

Activity: Upload Experimental Data to the CDC-MADB

The Upload feature provides the capability to view and analyze a specific data set. The link for uploading data is located on the Top Level Analysis Selection screen. Under the Links for data uploading heading, click the Upload link.

It is possible to be an authorized user on the system and not have been granted upload access, in which case the following message will appear, “You are not authorized to Upload data. Please contact your Systems Administrator.” A link is provided for convenience.

Submit Experiment Data Window

Navigating the Submit Experiment Data Window

FIG. 62 is a screenshot of the submit experiment data tool.

In order to submit experimental data you must have already created a Project (see the Creating a New Project Activity). Once a Project has been created, one or more experiments with the same print slide layout can be submitted to the project.

To submit experiment data:

1. Ensure that the radio button Dual Probe Ratio Data is selected.

2. Select an existing project from the drop-down list.

3. Click Continue to proceed.

Experiment Information Window

Navigating the Experiment Information Window

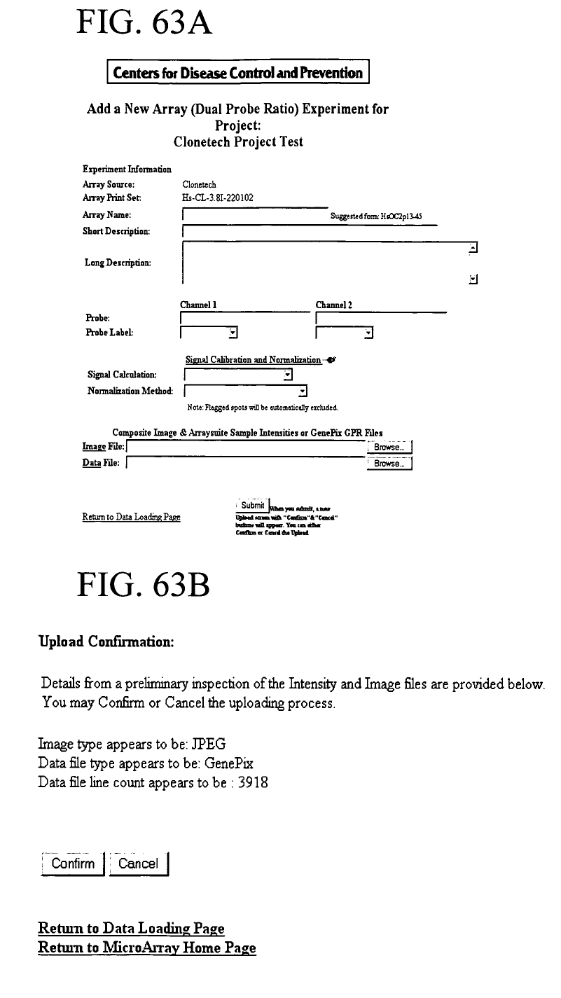

FIG. 63A is a screenshot of the Add a New Array Experiment Information window.

When submitting a new experiment to the CDC-MADB database, three types of information will be used to identify and describe your experiment.

1. Experimental description information

2. Image file name

3. Experimental data file name

Each of these data types will be captured through the web interface. The following are brief descriptions of the fields used to describe your experiment. All fields, except for the Long Description, are required for creating a project.

Array Source: This is the name of the array manufacturer. This information is automatically entered based on the values chosen from the Create New Project screen.

Array Print Set: This is the unique identifier supplied to you from your array manufacturer. This information is automatically entered based on the values chosen from the Create New Project screen.

Array Name: Use this text box to identify an experiment name. It is recommended that you give this some thought if you are expecting to have a number of experiments in your project. A standard naming convention can help you quickly identify your experiments. One such convention is to begin the name of the experiment with part of the Array Print Set Identifier. This text box is limited to 36 characters. An example might be “4 at 6 Hrs”.

Short Description: This text box is limited to 64 characters and is used as a column header to designate your experiment in a multi-experiment analysis tool.

Long Description: Use this text field to describe in more detail experimental information needed for clarification by others/collaborators who potentially may be sharing your data. This text box is limited to 255 characters, and is optional.

Probe: A name for each labeled probe can be entered in these text boxes. These fields are limited to 64 characters. An example of a probe name might be: “01control” or “ko-3hr.”

Probe Label: Select the dye label from the drop-down list.
Note: Your submission will be rejected if these values are the same for each channel.

Signal Calculations: Select one of the options to calibrate (or standardize) signal intensities. The options are:
Mean Int−Med Bkg
Above background by 3 SDs
Above background by 2 SDs

Normalization Method: Select one of the options to normalize the data. The options are:
Median (Ratio of Medians)
75^{th} percentile (Ratio of Medians)
Median (Ch Mean)
75^{th} percentile (Ch Mean)
Lowess (Ch Mean)
Lowess Sub-Grid (Ch Mean)

Values are automatically entered based on the values chosen from the Create New Project screen.

Experimental Data Input is captured by interactively uploading file information to the database. To upload your experimental image and data files:

1. Click the Browse button to search for your Experimental Image File on your computer file system.

2. Select the file to upload from the list.

3. Click the Open button. This will automatically indicate the path to your file within the Image File text box.

4. Repeat steps 1-3 to locate your Data File.

5. Click Submit to upload your data.
Note: The Image File and Data File fields must not be empty or you will receive an error message.
Note: The data file is the text file that contains the array data in a tabular format. The image file is the image of the scanned array. The image file must be in the format JPEG (.jpg).

If the system has successfully captured your data, then a screen similar to that shown in FIG. 63B will appear.

This confirmation will attempt to:
Evaluate the uploaded files
Determine the image file format (JPG)
Determine the approximate number of lines in the data file.

To accept this confirmation and continue with the upload process, press the Confirm button. To cancel this upload, press Cancel.

To add an experiment to a different project, click the Return to Data Loading Page link.

To return to the main page, click the Return to MicroArray Home Page link.

Activity: Check the Status of Web Uploads

This page is accessed from the Top Level Analysis Selection web page and provides a status report of successful arrays uploaded by the current user. This page will refresh every ten minutes.

Other Microarray Web Upload reports are available for viewing from this page. These include:
Summary by month of arrays uploaded in the past year
Daily summary of arrays uploaded in the past 90 days
Detailed listing of arrays uploaded within the past 7 days
Detailed listing of all uploaded arrays.
   Activity: Project Summary Report

The Project Summary Report is a reporting tool that provides a statistical summary of all experiments in a project, with normalization factor, mean signals, median backgrounds, signal/background ratios, % of features found, and description of the labeled probe.

A project to which at least one experiment has been submitted must be selected before the Project Summary Report tool can be selected.

6. From the CDC-MADB screen, select the Gateway link.
Note: To access this web site you must be a registered user and have a user login name and password.

7. The Top Level Analysis Selection screen is displayed.

8. Select a Project from the Project drop-down list.

9. Select Project Summary Report from the Analysis drop-down list.

10. Click Continue.

11. The Project Summary page is displayed.

Project Summary Report Window

Navigating the Project Summary Report Window

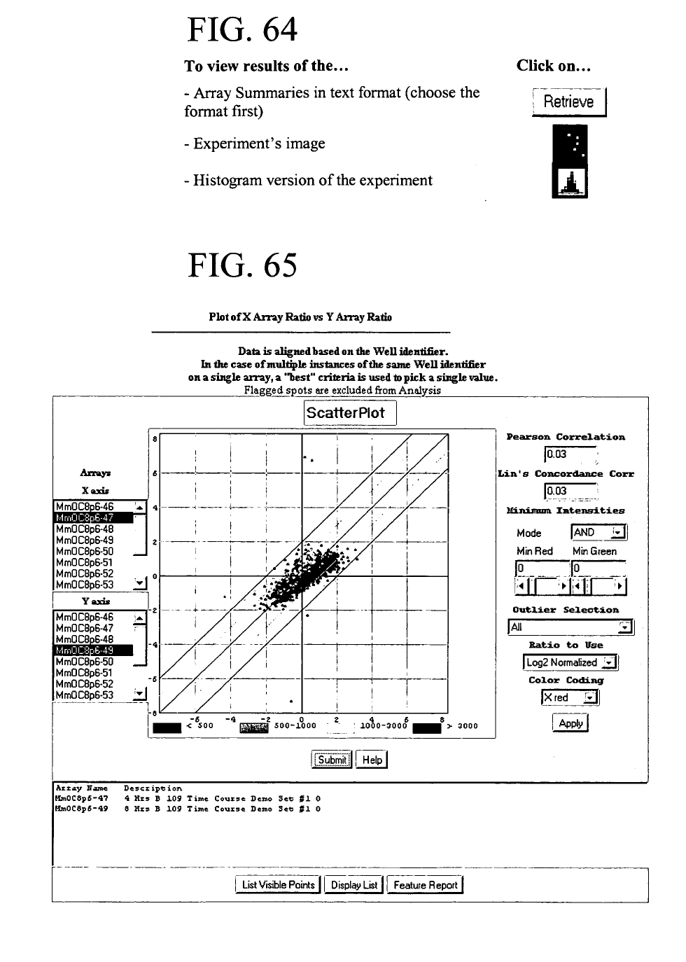

The data results displayed on the Project Summary web page can be viewed by three different means: text, spot images, and histograms. Examples of the results are shown in FIG. 64.

Results Display

To change the size of the experiment's image, choose the desired scale from the drop-down list and then press the Resize button.

Spot Image

FIG. 45A is a screenshot of the spot image.
Note: In the system, this image can be resized to allow users to view the entire image or zoom into a specific area.

Histogram

FIG. 45B is a screenshot of a histogram of the image data.

The Histogram provides a visual chart of the image data.

If you wish to acces this data as a text file, choose the format from the drop-down list, and then press the Retrieve button.

From this screen you may change the bin size which will refresh the display. The bin size determines the resolution of the plot. This means that each log unit is divided into a specified number of subunits of intensity values. Once the bin size is determined for each bin location, the number of genes that fit the value is determined and vertical lines are drawn at bin locations depicting the relative count with respect to the max count shown on the Y axis.

Use the drop-down list to select the bin size. The Histogram will be redrawn at the new resolution. The default bin size is 40.

Printing Internet Pages

Many of the File and Edit menu items in Internet Explorer work as they do in other applications.

To print the contents of the current page, do one of the following:

3. From the File menu, choose Print.

4. Click the Print button in the toolbar.

Depending on your browser's options, a dialog box may appear allowing you to select different printing options.

In Internet Explorer, you can choose Print Preview from the File menu to see a screen display of a printed page.

Activity: Analyze the CDC-MADB Data

Overview of Analysis Tools and Approach

A number of powerful analytical and visualization tools are included in the CDC-MADB system. Detailed descriptions for these tools are provided in the appropriate sections of the manual. A brief summary of these tools is provided here.

7. Scatter Plot Tool: Provides an interactive scatter plot of gene expression intensities for any pair of experiments; allows color-coding of gene intensities and subsetting capabilities.

8. Java Experiment Array Viewer: The Java array viewer is available for both single and multi experiments. These tools were designed to be an intuitive and efficient way to gather significant information from hybridization data.

9. Ad Hoc PID Query: Provides extensive search and subsetting capabilities. For each array that satisfies a query, the experiment's image and histogram of the gene expression intensities are provided. Genes that satisfy query criteria can be clustered. Hierarchical clustering, Kmeans clustering, or Self-Organizing Maps (SOM) clustering algorithms are available. Results can be either viewed online or retrieved.

10. Ranking Display Tools: Ranking display tools for both single and multi experiments designate baselines for against which other experiments will be ranked. These tools were designed to help investigators quickly rank and sort various experimental data.
Note: More details about these analysis tools are available in later chapters of this user manual.
   Filtering and Retrieving Data Sets

A comparison analysis of the gene expression profiles between healthy subjects and subjects with a disease is the main goal of the CDC-MADB system. To perform this task, subgroups of experiments related to particular groups of subjects are queried from the system. Examples of group definitions are given below:
Subjects from Atlanta Study; 30-40 years old; white; males; controls.
Subjects from Atlanta Study; 30-40 years old; white; males; with long history of CFS (chronic fatigue syndrome).
Subjects #1, 3, 8 from Atlanta Study.

Each query results in a data set that contains gene expression profiles for a particular group of samples. From this sample group, existing CDC-MADB analysis tools can be launched to investigate corresponding microarray results.

Statistical Analysis of Microarray Data

The following approaches to getting started with microarray analysis are suggested. Some of these analytical techniques are currently available in the CDC-MADB system while others may require additional tool sets. Export of data is provided to support these recommendations.

Preprocessing:
Normalization
Imputation of missing values
Subsetting based on percent of missing data or significance of the gene expression difference

Visualization:
Gene expression distributions
Quantile-Quantile plots
Scatter plots

Group Comparison and Discriminant Analysis:
Visual comparisons via scatter plots
Principal component analysis
Multi-Dimensional Scaling
Visual exploratory analysis of correlation matrix
Discriminate analysis
Significance tests (t-test, paired t-test, F-test), validation via permutation tests

Group Discovery and Cluster Analysis:
Hierarchial clustering
Kmeans clustering
SOM clustering

Many of these tools are implemented in the CDC-MADB system. At the later stages, more sophisticated methods can be added. Meanwhile, export capabilities are provided to facilitate data analysis using external software packages.

Chapter 5. Visualization Tools

Introduction

Visualization tools are primarily used to quickly view trends in the data.

These trends can be depicted graphically or in more complex images such as dendrogram tree structures or 3-D rotating figures. There are four different visualization tools from which you may choose to graphically plot the findings:
Scatter Plot
Java Single Experiment Array Viewer
Java Multi Experiment Array Viewer
M vs. A Plot
   Scatter Plot

This applet is a simple visualization and analysis tool for formatting microarray experiment data into a scatter plot. It is designed for analyzing a pair of related experiments. The actual values used for drawing the plot are the raw (scaled) intensities and the log2 normalization of each clone, assuming that the two experiments have the same number of clones in the same order.
Note: On the scatter plot, the intensity instead of the log intensity is labeled on the marked ticks.
   Selecting the Scatter Plot Tool

FIG. 65 is a screenshot of the Scatter Plot tool of the Dual Probe system.

A project to which at least one experiment has been submitted must be selected before the Scatter Plot tool can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select Scatter Plot Tool, from the Analysis drop-down list.

5. Click Continue.

6. The Scatter Plot Tool screen is displayed.

Scatter Plot Tool Window

Navigating the Scatter Plot Tool Window

To begin, review and select the Scatter Plot attributes:

1. Experiments: Select experiments from the left of the scatter plot field, labeled “X axis” and “Y axis.” An experiment selected from the “X axis” list will have its data mapped on the horizontal axis, while an experiment selected from the “Y axis” list will be plotted on the vertical axis.

2. Minimum Intensities: These fields are labeled Min Red and Min Green and are found to the right of the scatter plot field and there are two ways to specify the Minimum Intensity: 1) typing the minimum intensity value in the labeled field, or 2) sliding the scroll bar underneath the field to increase or decrease the value. To specify values greater than the maximum values of the scroll bar, type the value directly into the text field. The Minimum Intensity will apply to both experiments. The Mode switch specifies whether the minimum intensities for the red and green channel apply independently or together. “AND” means that a data point has to be above both thresholds in order to be included. “OR” means that a data point will be included if it is above either one of the thresholds. For ordinary use, “AND” should be selected.

3. Ratio To Use: The application can use Log2 Normalized or Raw (Scaled) ratios to draw the scatter plot. The default is Log2 Normalized. The X and Y axis will change depending upon the option selected.

4. Color Coding: To provide a better distinction among the scatter plot data, each data point will be colored based on its intensity values.

5. The Pearson Correlation Coefficient will be calculated each time the Submit button is pressed. Its value is based on the actual normalized data points regardless of whether it is currently being displayed on the scatter plot or not.

6. Lin's Concordance Correlation will be calculated each time the Submit button is pressed. Its value is based on the actual normalized data points regardless of whether it is currently being displayed on the scatter plot or not.

7. Outlier Selection: These five options: All, Above four fold, Above two fold, Below negative two fold, and Below negative four fold, determine which clones are displayed in the ScatterPlot.

8. The Submit button must be pressed every time you change an experiment so that the data can be updated and redrawn. The first time you click Submit, it may take several minutes to download the experimental data from the database. However, once the experiment data are loaded and you wish to change only the attributes, click the Apply button. This update will be much faster.

Once the data have been plotted, further analysis can be executed with individual or multiple clones. To select clones from the Scatter Plot field, simply click and drag your mouse across the clones in which you are interested. (The screen area will highlight and change color to designate the selected area.) You may select single or multiple clones depending on how many points are within your selection area. Once a clone or a group of clones have been selected:

9. Click the Display List button to view details on the clones within the selection area. (This data will appear in the field below the Scatter Plot as well as in a separate window).

10. Click on a clone in the field below the Scatter Plot and then click on the Feature Report button to retrieve detailed information about that particular clone. When the Feature Report is returned, hyperlinks to related URLs appear in the report. Move your mouse cursor over the report to determine which elements have links. (Usually, these links are noted by colored text.) Click the link for more details.

11. Click the List Visible Points button to view a list of all the clones currently visible on the Scatter Plot. This list appears in the field below the Scatter Plot.

12. The plotted data can also be retrieved in text format. To do this, select the desired format from the drop-down list in the separate window that was launched when you clicked the Display List button and click the Retrieve button. The data are now displayed as text in the specified format.

Java Single Experiment Array Viewer

The Java Array Viewer is designed to be an intuitive and efficient way to gather significant information from an individual hybridization experiment.

Selecting the Java Single Experiment Array Viewer Tool

A project to which at least one experiment has been submitted must be selected before the Java Single Experiment Array Viewer can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select Java Single Experiment Array Viewer, from the Analysis drop-down list.

5. Click Continue.

6. The Single Array Viewer Tool is displayed.

7. Select an Array to view from the drop-down list.

8. Click Continue.

9. The Single Array Viewer Tool histogram is displayed.

Java Single Experiment Array Viewer Window

Navigating the Java Single Experiment Array Viewer Window

The first page of the Array Viewer shows a histogram of the red/green ratios of the data from one experiment as shown in FIG. 48. By default, in the current implementation, flagged spots are excluded. Flagged spots include: Empty, Control, either no Red or Green Target detected and user flagged problem spots.

To query, review and select the query options:

1. Selector Type: One of four methods can be used to query the data using the histogram: Confidence, Less Than, Range, and Greater Than. Each of these four queries can also be limited by various restrictions. A Minimum Intensity can be set so that only clones that have a red AND a green intensity above this lower limit are returned. A Maximum Intensity can be set so that both the red AND green intensity must be below this upper limit. Minimum Size limits clones to those that have both a red AND a green pixel size above a minimum value. Title Keyword restricts the returned clones to only those that have the keyword in their title
Confidence: When this option is chosen, the histogram shows two gray vertical lines that show the upper and lower confidence value for that particular experiment. The initial confidence percentage is set at 99.0%. This value can be edited in the Confidence % field. In order for the new setting to be registered and affect the query, the Set Confidence button must also be clicked.
Range: When this option is chosen, the gray confidence lines are replaced with a pair of blue lines which can be repositioned by clicking the mouse inside the histogram window. The line being repositioned toggles with each mouse click.
Less Than: When this option is chose, the gray confidence lines are replaced with a single blue line, initially positioned at the high confidence mark, which can be repositioned at the high confidence mark, which can be repositioned by clicking the mouse inside the histogram window.
Greater Than: When this option is chosen, the gray confidence lines are replaced with a single blue line, initially positioned at the high confidence mark, which can be repositioned by clicking the mouse inside the histogram window.

2. Submit Query:
Clicking on Submit Query button activates your query. This will automatically return all the clones with a ratio in between those two blue lines positioned on the histogram. When either Greater Than or Less Than is selected, only one line appears for positioning on the histogram. Submit Query returns all the clones Greater Than or Less Than the positioned value. (See below for more information on the Results Window.) Lastly, on the main page, selecting View Slide will launch the Results Window with no returned clones, but allows you to visually pick a clone on the image and get the hybridization information.
   Results

The Results Window is divided into two sections to display the returned clone information. The top window displays a JPEG image of the hybridization. When a clone is returned after a query it is boxed with either a red or green box and a number to reference it to the quantitative data. The lower window shows the quantitative data on each clone. Each row is one particular clone with the following information in each subsequent column. The first column is an index which references the clones to the boxes highlighting the spots in the upper window. The second column shows the internal database clone ID, followed by Ratio Value, Red Intensity, Green Intensity, the number of Red Pixels, the number of Green Pixels, and the title.

After a database query, the information is sorted by ratio values from lowest to highest. The lower window is also linked to more information. By clicking on the red counter number, a new window is launched that shows a zoomed in view of the particular clone and repetition of the information. By clicking on the blue clone ID, a comprehensive Feature Report will be displayed in another browser window.

There are several options listed on the bottom of the results window.
Close Frame after new Query: This checkbox is default checked, which means that after a new query on the main page this window will close. If unchecked this window will not close after a new query.
Allow Clone Selection: This checkbox, when selected, will allow you to click on the upper window JPEG and get the hybridization information about particular clones. This is default checked only when you click View Slide; otherwise, it is default unchecked.
Clear List: This button will purge the list of clones returned by a query and/or manually selected.
Display List: This button will result in the list being displayed in a browser window. From there, you can save or print the list. A pathway is not yet fully implemented.
   Java Multi Experiment Array Viewer

The Array Viewer is designed to be an intuitive and efficient way to gather significant information from hybridization information.

Selecting the Java Multi Experiment Array Viewer Tool

A project to which at least one experiment has been submitted must be selected before the Java Multi Experiment Array Viewer can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select Java Multi Experiment Array Viewer, from the Analysis drop-down list.

5. Click Continue.

6. You will be prompted to log in to the system again.

7. The Multi Array Viewer Tool screen is displayed.

Java Multi Experiment Array Viewer Window

Navigating the Java Multi Experiment Array Viewer Window

FIG. 49 is a screenshot of the Multi Experiment Array viewer.

The Multi Array Viewer is divided into three sections.

1. The Control panel allows you to select and filter query criteria.

2. The Display panel displays the plot of the experimental data.

3. The Detail panel displays the quantitative information of the clone.

To develop a query, review and select the desired attributes:

1. Select an experiment from the control panel: Ratio Outside, In Arrays, Mean Intensity, Spot Size or Keyword.

2. Once the attributes are set, press the Submit Query button to query the data and determine all the clones that meet the ratio criteria and meet the filter requirements. It will then return the ratios for that clone in all the selected experiments and draw a plot in the Display panel.
Note: Query times average around 10-15 seconds. Please be patient.

Also be sure that all selected experiments are from the same print, so that spots across slides correspond.

This display can be displayed in scales. The Y-axis can either be a straight linear progression from 0 to the selected ratio range. (Default is 10). Or the Y-axis can be the log base 2 of the ratios.

In the large display of the clone data, you can click on a particular spot, and see the ratio of the specified clone across all the selected experiments. An Applet window will be launched that displays additional information about the clone across the selected experiments and also, the quantitative data will be highlighted in the lower display. This can be accomplished also by clicking on the “#” of a clone in the lower display. The Applet window will be launched and the ratio trend will be shown in the large display window.

Lastly, the Clone_Id, which appears in the Detail panel, is hyperlinked to the Clone Feature Reports which are linked to other value-added information sources.

M vs. A Plot

The data on an M vs. A Plot are aligned based on the Well Identifier. In the case of multiple instances of the same Well Identifier on a single array, a “best” criterion is used to pick a single value.

Selecting the M vs. A Plot Tool

A project to which at least one experiment has been submitted must be selected before the M vs. A Plot Tool can be selected.

1. From the CDC-MADB screen, select the Gateway link.
Note: To access this web site you must be a registered user and have a user login name and password.

2. The Top Level Analysis Selection screen appears.

3. Select a Project from the drop-down list.

4. Select M vs. A Plot, from the Analysis drop-down list.

5. Click Continue.

6. The M vs A Plot Tool screen is displayed.

M vs. A Plot Tool Window

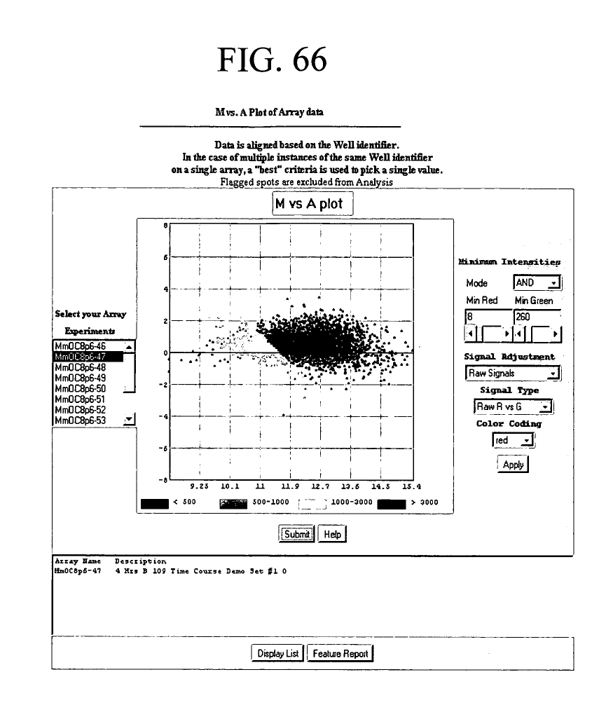

FIG. 66 is a screenshot of the M vs. A plot tool.

Navigating the M vs. A Plot Tool window

To begin, review and select the plot attributes:

1. Experiments: Select an experiment from the Experiments list to the left of the M vs A Plot field.

2. Minimum Intensities: There are two ways to specify the Minimum Intensity for the red or green channel: 1) typing the minimum intensity value in the labeled field, or 2) sliding the scroll bar underneath the field to increase or decrease the value. To specify values greater than the maximum values of the scroll bar, type the value directly into the text field. The Mode switch specifies whether the minimum intensities for the red and green channels apply independently or together. “AND” means that a data point has to be above both thresholds in order to be included. “OR” means that a data point will be included if it is above either one of the thresholds. For ordinary use, “AND” should be selected.

3. Signal Adjustment: Raw Signals or Signal−Background.

4. Signal Type: Raw R vs. G, Normalized 50%, or Normalized 75% may be selected.

5. Color Coding: To provide a better distinction among the scatter plot data, each data point will be colored based on its intensity values. Because each data point contains four different intensity values, you can determine which channel to use for color-coding.

6. The Submit button must be pressed every time you change an experiment so that the data can be updated and redrawn. The first time you click Submit, it may take several minutes to download the experimental data from the database. However, once the experiment data are loaded and you wish to change only the attributes, click the Apply button. This update will be much faster.

Once the data have been plotted, further analysis can be executed with individual or multiple clones.

7. To select clones from the M vs A Plot field, simply click and drag your mouse across the clones in which you are interested. (The screen area will change color to designate the selected area.) You may select single or multiple clones depending on how many points are within your selected area. Once a clone or a group of clones have been selected, click the Display List button to view details on the cloned area. (This data will appear in the display area below the M vs A Plot field, as well as in a separate window.)

8. To view the Feature Report, select the clone from the list in the display area below the M vs A Plot field and click the Feature Report button. When the Feature Report is returned, hyperlinks to related URLs appear in the report. Move your mouse cursor over the report to determine which elements have links. (Usually, these links are noted by colored text.) Click the link for more details.

Chapter 6 Retrieval and Filtering Tools

Introduction

Retrieval and filtering tools function to bring back specific subsets of data based on the nature of the data. Filtering tools use the characteristics of the data to define a range of interests and retrieval brings back and presents the results. These tools are extremely useful in creating sets of data that contain high value information. Many of these data sets can be saved and imported into supplemental analysis tools.

These are searching tools that query a number of experiments for specific gene information.

Selecting Retrieval or Filtering Tools

A project to which at least one experiment has been submitted must be selected before either the Ad Hoc PID Query or the 1 or 2 Group Logic Retrieval Tool can be selected.

1. From the CDC-MADB screen, select the Gateway link.
Note: To access this web site you must be a registered user and have a user login name and password.

2. The Top Level Analysis Selection screen is displayed.

3. Select a Project from the Project drop-down list.

4. Choose the desired query tool (Ad Hoc PID Query or 1 or 2 Group Logic Retrieval) from the Analysis drop-down list.

5. Click Continue.

6. The Ad Hoc PID Query or 1 or 2 Group Logic Tool screen is displayed.

Ad Hoc PID Query

Overview

The Ad Hoc PID Query is a searching tool that queries a number of experiments for specific gene information. This tool was designed to help investigators quickly monitor genes of interest and to provide a visual display of the queried information.

Ad Hoc PID Query Window

Navigating the Ad Hoc PID Query Window

There are four areas on the Ad Hoc Query Tool Form screen in which you can enter data query criteria. An overview of the steps for completing a query appears below, with detailed descriptions of each screen option provided later in this chapter. These sections are:
Spot Filter Options
Gene Selection Criteria
Format/Preview Options
Array Selection

To begin, review and select the query options:

4. Select the desired Signal Intensity/Background.

5. Select the desired Spot Size and Signal.

6. Choose whether to exclude Bad or Bad or NF spots.

7. Choose the Gene Selection Criteria from the drop-down list and enter a relative value in the blank field.

8. Choose the desired format for the returned results.

9. Check the Use Names in Preview box to display the array names in the Preview Table.

10. Check the Show Spot Images box to display the spots in the Preview Table.

11. Choose how the returned results are to be ordered with the Order by drop-down list.

12. Select the desired arrays for query using the radio buttons.

13. When all information is selected, click the Submit button. (The View Array Results section explains how the data is displayed.)

Spot Filtering

Individual array spots can be filtered for spot quality by a number of criteria, to allow those spots greater than or equal to the selected value to pass the filter.

FIG. 53A shows a screenshot of the spot filtering tool of the Ad Hoc PID Query.
Signal Intensity/Background: This filter simply dictates how strong the signal intensity should be vs. the background intensity for each spot. (Default 0.0)
Spot Size: The percentage of feature pixels with intensities more than one standard deviation above the background pixel intensity at respective wavelength.
Signal: This filter sets the minimum absolute intensity of the signal.
Exclude Spots Flagged: This drop-down list presents two options. Bad spots are spots flagged through visual examination of the spot image. NF indicates that the image analysis program does not find the spot.
   Gene Selection Criteria

Extract array data by searching with one of the Query categories.

FIG. 53B shows a screenshot of the gene slection tool of the Ad Hoc PID Query.
Putative ID (PID) like: The PID is a single derived description in order of preference (1) local annotation, (2) 5′ UniGene title, (3) 3′ UniGene title and (4) Unknown. This search expects a character string. The search uses wild cards to find any PID that contains the query string in it. Use a leading space to force the match to the beginning of words only or a trailing space to force the match to the end of words only. Using both a leading and trailing space will match only full words. The search is case insensitive.
   Examples:
   “APO” would match Apoptosis or hepapoietien
   “APO” would match Apoptosis but would not match hepapoietien
SwissProt ID: This option is an annotated protein sequence database. This search expects a character string. The search uses wild cards to find any Unigene Title with the query string in it. Use a leading space to force the match to the beginning of words only or a trailing space to force the match to the end of words only. Using both a leading & trailing space will match only full words. The search is case insensitive.
LotusLink ID: This option provides a single query interface to curate sequence and descriptive information about genetic loci. It presents information on official nomenclature, aliases, sequence accessions, phenotypes, EC number, MIM numbers, UniGene clusters, homology, map locations, and related web sites. This search expects a character string. The search uses wild cards to find any Unigene Title with the query string in it. Use a leading space to force the match to the beginning of words only or a trailing space to force the match to the end of words only. Using both a leading and trailing space will match only full words. The search is case insensitive.
GenBank ID: This is an NIH genetic sequence database, an annotated collection of all publicly available DNA sequences. This search expects a character string. The search uses wild cards to find any Unigene Title with the query string in it. Use a leading space to force the match to the beginning of words only or a trailing space to force the match to the end of words only. Using both a leading and trailing space will match only full words. The search is case insensitive.
Inventory Well ID is: This searches the list of Well identifiers. This search requires a number. The search performs an exact match.
   Examples:
   455
      Format/Preview Options

These options control the format of the returned results. Use the drop-down lists to view all available options. The data returned are always based on the normalized (calibrated) ratios.

FIG. 53C shows a screenshot of the Format/Preview Options screen of the Ad Hoc PID Query.

Results Format: This drop-down menu allows you to choose how you want the results returned and displayed.
HTML Preview: The results are returned in a web browser.
Eisen Cluster: The results are returned as a file, formatted for direct input to the Eisen/Stanford Cluster program. It is recommended that you save this as a text or “*.*” file with a “.txt” extension. The data values returned for this format are the LOG base 2 of the normalized intensities.
PC, Macintosh and Unix: The results are returned as a TAB delimited text file formatted for the appropriate operating system. The results include a header portion describing the arrays selected and the query.
MS-Excel: The results are returned as MS-Excel content.

Order by: A variety of options can help determine the order in which the data are returned.

Limit Preview: This option limits the number of output rows displayed in the browser, with a default setting of 25 rows. It should be noted that this menu only affects data displayed in the browser; data exported to a tab-delimited file, Eisen Cluster format, or an Excel spreadsheet is always returned in their entirety.

Checkboxes:
Use Names in Preview: Checking the box will display the names of the selected arrays in the web browser. If not checked, then only the selected array number is displayed above the data in the Preview. It is generally recommended that you leave this box unchecked.
Show Spot Images: Checking the box will display an image of each spot, if available.

CAUTION: This option is highly memory-intensive and is only recommended for checking spot quality when necessary. Checking this box will substantially slow the display of results, particularly on low-bandwidth connections such as those found with a dial-up modem. Each image takes time to be rendered by the web browser.

Array Selection

This section of the Ad Hoc Query tool allows you to select the Arrays to be analyzed.

FIG. 53D shows a screenshot of the array selection tool of the Ad Hoc Query.
Selecting Arrays: There are three selection columns to the left of the Array Name & Description list. Initially, the first column (under the “-” button) is selected for all arrays. An array is de-selected when the radio buttons in this column are selected. To select individual arrays for analyzing, click the radio button in the “A” column.
Using Button Shortcuts: The “-” and “A” buttons at the top of the column work in the following manner. Clicking on the “-” de-selects all arrays. Clicking on the “A” selects all arrays. Individual arrays can still be de-selected by clicking the radio button in the “-” column.
   Note: To function, these buttons require a JavaScript enabled browser.
Reciprocal Ratios: If the “I/R” column is checked for a selected array, then the reciprocal ratio for that array is used in the analysis.
   Submit

When all filters are set to your satisfaction, click the Submit button to activate the tool. For convenience, a Submit button is located at the top and bottom of the Array Selection panel, as well as at the top of the form.

Query Execution

If execution of the query exceeds 20 seconds, an interim page will be displayed indicating that the query is still proceeding. In Internet Explorer, dots will be displayed every few seconds as an indication that the system is still working. When the query is complete, press the Continue button to retrieve the results. The results page is held in a temporary file cache and can be bookmarked for later retrieval.

Results

The returned results will be similar to the example shown in FIG. 69, depending on the options specified on the previous screen.

Press the View button at the top of the results page to launch the Array Summaries tool in a separate window. Beneath that is a listing of the arrays placed on the form into group A Below each array listing is a summary of the returned results, indicating how many rows met the specified criteria and repeating the criteria used on the form.

Many URLs related to this query will appear in the returned results. Move your mouse cursor over the screen to determine which elements have links. (Usually, these links are noted by colored text.) Click the link for more details.

To the left of each array description are icons to allow viewing the array composite image, or to allow viewing a histogram of the normalized ratios of that array. These icons are shown in FIG. 64. These results can be displayed graphically, by clicking on the button to the left of the array. (See Chapter 3, Project Summary Report, for a more detailed look.)

Server Side Clustering

Clustering and visualization of the clusters has been implemented using modified versions of Gavin Sherlock's Xcluster program and SOMviewer and makeCluster viewer programs developed at Stanford University.

There are three types of clustering options available to you to help with your analysis: Hierarchial Clustering, Kmeans Clustering, and SOM Clustering. The results displayed will depend on the type of clustering program invoked

To begin, review and select the clustering steps and options:
1. Select the desired clustering tool.
2. Select the desired options.
3. Click the Cluster button.
4. Your clustered results will be displayed.

1. Hierarchical Clustering: Specify the parameters that control the hierarchical clustering. The Hierarchical Clustering Options Tool is shown in FIG. 55.
Genes & Arrays: The following options can be selected from the associated drop-down lists.
   Not Clustered: Choosing this will disable the hierarchical clustering of Genes and/or Arrays.
   Non-centered Metric: Uses a non-centered metric.
   Median Centered Metric: Use a centered metric.
Distance Metric: The following options can be selected from the associated drop-down lists.
   Pearson Correlation
   Euclidean Distance
Name (optional): If you enter a name, it will be used to “tag” your files on the server rather than the server generated tag. This can be handy in managing files you may retrieve with Treeview. The server names will be your MADB login combined with a date/time field.

2. Kmeans Clustering: Specify parameters that control the partitioning of the Kmeans Clustering. The Kmeans Clustering Tool is shown in FIG. 56.
Number of Nodes: The drop-down list allows you to choose from 2 to 15 Nodes.
Maximum Number of Iterations: The drop-down list allows you to select from a range from 25 to 250 the maximum number iterations. Generally, the Kmeans clustering will converge before the maximum number of iterations is reached.

Kmeans node clustering options: User can specify parameters that control the hierarchical clustering of the individual Kmeans nodes.
Genes & Arrays: The following options can be selected from the associated drop-down lists.
   Not Clustered: Choosing this will disable the hierarchical clustering of Genes or Arrays within each Kmeans node.
   Non-centered Metric: Uses a non-centered metric.
   Median Centered Metric: Uses a centered metric.
Distance Metric: The following options can be selected from the associated drop-down lists.
   Pearson Correlation
   Euclidean Distance
Name (optional): If you enter a name, it will be used to “tag” your files on the server rather than the server generated tag. This can be handy in managing files you may retrieve viewing with Treeview. The server names will be your MADB login combined with a date/time field.

3. Self Organizing Maps (SOM) Clustering: You can specify parameters which control the partitioning of the 2-dimensional SOM and whether to seed the initial SOM vectors with random numbers. The program currently screens out any Genes whose max(intensity)/min(intensity) across the arrays is <2.

The SOM Clustering Tool is shown in FIG. 57.
X & Y Dimensions: The drop-down list lists allow you to choose an X and Y dimension between 1 and 15.
Number of Iterations: Select the number SOM iterations from a range of 50000 to 250000 from the drop-down list. An iteration is picking a Gene at random and modifying the SOM vector which most closely matches the Gene expression and the neighboring SOM vectors.
Initialize with Randomized Partitions: When checked, the initial SOM vectors will be initialized with random numbers.

SOM element clustering options: User can specify parameters that control the hierarchical clustering of the individual SOM elements.
Genes & Arrays: The following options can be selected from the associated drop-down lists.
   Not Clustered: Choosing this will disable the hierarchical clustering of Genes or Arrays within each SOM element.
   Non-centered Metric: Uses a non-centered metric.
   Median Centered Metric: Uses a centered metric.
Distance Metric: The following options can be selected from the associated drop-down lists.
   Pearson Correlation
   Euclidean Distance
Name (optional): If you enter a name, it will be used to “tag” your files on the server rather than the server generated tag. This can be handy in managing files you may retrieve with Treeview. The server names will be your CDC-MADB login combined with a date/time field.
   Server Side Clustering Results

The data are clustered and the results are returned in a separate window. Click the View Clusters button for a more detailed look at the clustering results. Once the results are displayed, use the features below to guide your interests in seeing the results.

1. To view the text results on your PC, left-click either the C or G character above the image. A separate window appears displaying the data.

2. To save the results on your PC, right-click either the C or G characters above the image, and choose Save As. Choose the specified path in which to save the file and it will be downloaded.

3. Click on the Thumbnail cluster image to display an expanded image view. Once in the expanded view, you may click on the clone line to generate a Clone report, or click on the pattern line to generate a collage of Spot images.

Chapter 7 Ranking Tools

Single Rank/Multi Display

The Single Rank/Multi Display is a ranking tool that designates one experiment as a baseline upon which all other selected experiments will be ranked. This tool was designed to help investigators quickly rank multiple experiments based on a single experimental datum and to provide visual information for publications.

Prior to Running Single Rank/Multi Display

A project to which at least one experiment has been submitted must be selected before the Single Rank/Multi Display tools can be selected.

1. To launch, enter through the CDC-MADB Gateway link.

2. Choose a Project from the Projects drop-down list.

3. Choose Single Rank/Multi Display from the Analysis drop-down list.

4. Click Continue.

5. The Single Rank/Multi Display screen is displayed.

Navigating the Single Rank/Multi Display Window

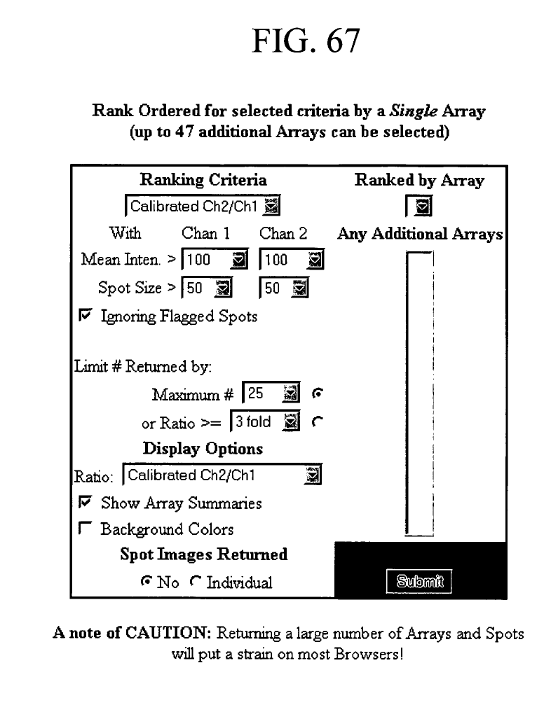

A screenshot of the Ranking tool is shown in FIG. 67.

The Single Rank/Multi Display query form captures three types of information:
Ranking Criteria
Experiments to be ranked
Display options

To begin, review and select the ranking tool options:

1. Ranking Criteria can be chosen from the drop-down list. The options are Calibrated Ch1/Ch2 and Calibrated Ch2/Ch1.

2. Mean Intensities for Channel 1 and Channel 2 can be chosen from the drop-down lists. These values indicate intensities greater than the values in the entry box, and reflect values above background. These values are usually set between 100 and 500 for each channel.

3. Spot Size can also be selected from the drop-down lists. Only spots with a size greater than indicated will be used in the ranking information. The number of undetected spots can affect this, because spot sizes of zero will lower the average. The average size of a spot is approximately 130 pixels using the ArraySuite (Yidong) software, and the minimum spot size is therefore usually set to 10-50 pixels.

4. Flagged Spots can be either included or excluded from the ranking. Checking this box will remove Flagged Spots from the ranking.

5. Limit # Returned by Maximum # or Ratio>=can be designated in the entry boxes to assign the number of rankings returned in the drop-down list.

6. Ranked by Array allows for the designation of the experiment to which all other arrays will be compared and ranked.

7. Multiple array experiments can be individually selected from the list box of Any Additional Arrays. Multiple array selections can be made while pressing and holding the [Ctrl] key while simultaneously selecting each array.

8. Click the Submit button to initiate the query.

Display options can be used to tailor your query outputs. The following list explains each option.

Ratio: The source of each ratio can be designated from the drop-down list provided.

Show Array Summaries: Check this box to display additional experimental summary information. See Results Display for an example of an Array Summary.

Background Colors: Check this box to display a false color scale designation for each ratio in the query results.

Spot Image Returned: Select these radio buttons to choose the type of spot displayed in the results table.
No: No image will be returned if this radio button is marked.
Individual: If by chance suspect artifacts need to be confirmed, individual spots which are cut out to show 50% of the neighboring spots will be returned. This will provide a better image of the surrounding area.
   Results Display

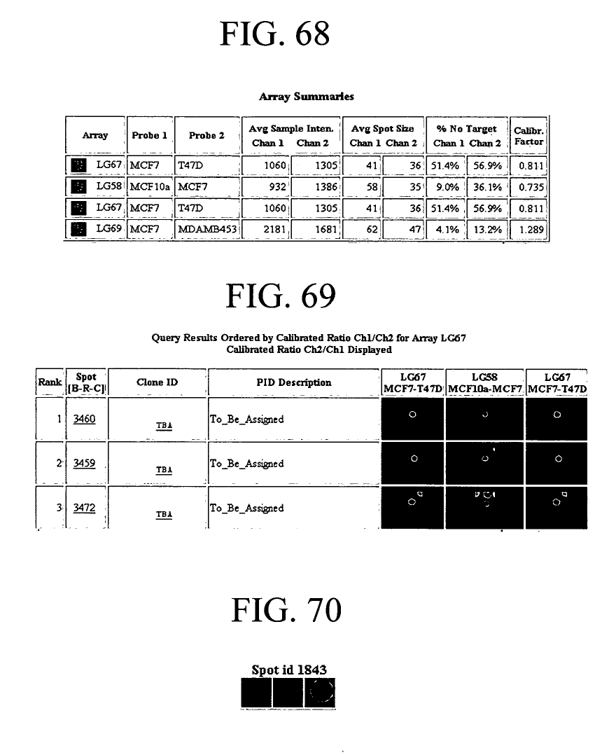

The Array Summaries table shown in FIG. 68 provides a quick glance of summary information about an entire experiment. This table shows information about:
Array: Information in this column is linked to an image of each experiment. If selected, a new browser page is launched and the experimental image data are returned. This image can be resized for viewing and capturing.
Probe 1: Shows the naming convention entered designated for probe 1.
Probe 2: Shows the naming convention entered designated for probe 2.
Average Sample Intensities for Channel 1 and 2: Shows the average mean intensity based upon values set on the Single Array Query form.
Average Spot Sizes for Channel 1 and 2: Shows the average mean intensity based upon values set on the Single Array Query form.
% No Targets for Channel 1 and 2: This value represents the percentage of spots not detected by the array software program. This value provides an estimate of quality for the experiment. A good experiment might have a normal of 1-10%. If this value is high, it may indicate that either the signal intensities for one channel or the other is low/weak or that a large area of the whole slide may have a problem. Visually inspecting the array image would be recommended to determine the meaning of these values.
Calibration Factor: This is a numerical value used to adjust the ratio of the experiment so that the median ratio is equal to one. In the normal distribution of ratios within an experiment, the median will not precisely equal one because of experimental error. This is used in all the tools, and factors greater than 4-5 are unacceptable. More often, the calibration factor ranges between 0.5-2.
   Note: You can disable the Array Summaries report from the Single Array Query form.

The Rank Order Query Results table shown in FIG. 69 ranks across experiments based on the ratio information showing the greatest change in ratio across the experiments

This ranking results table shows information about:
Rank: Information in this column is linked to an image of each experiment. If selected, a new browser page is launched and the experimental image data are returned. This image can be resized for viewing and capturing.
Spot [B-R-C]: The spot link will launch a new web browser page and display the spot images for the selected experiments that correspond to the Clone ID. For example, clicking on spot number 1843 from this column would return an image such as that shown in FIG. 70.
B-R-C stands for the Block-Row-Column location of the spot on the slide.
Clone ID: The Clone ID link will launch a new web browser page and display a Clone Report (see Appendix A). This report has specific clone information that is updated on a regular basis and is linked to a number of peripheral resources such as UniGene and GenBank. In addition, a direct link to the UniGene cluster information is provided (u), although this information is available in each clone report. For private clones the designation TBA (to be assigned) is used to indicate an incomplete clone report.
PID Description: This description is a simple annotation of the clone information and represents a putative ID of the clone. Typically, gene names or title information is provided. This information is currently captured from a variety of sources (see Appendix B).
Selected Experiments: Each of the queried experiments will be designated as a single column. The experiments are returned in the ranked order as compared with the designated single experiment from the query form. Options for viewing the spot image and background color can be selected from the query form. Ratio information is displayed below the spot image.
   Multi Rank/Multi Display

The Multi Rank/Multi Display is a ranking tool that uses criteria across an entire set of experiments for ranking. This tool was designed to help investigators quickly sort various experimental data by specific criteria such as intensity, spot size or fold difference in expression. The outputs provide visual information for initial evaluation and publication.

A project to which at least one experiment has been submitted must be selected before the Multi Rank/Multi Display tool can be selected.

1. You must enter through the CDC-MADB Gateway link.

2. Select a Project from the Project drop-down list.

3. Select Multi Rank/Multi Display from the Analysis drop-down list.

4. Click Continue.

5. The Multi Rank/Multi Display screen is displayed.

Navigating the Multi Rank/Multi Display window

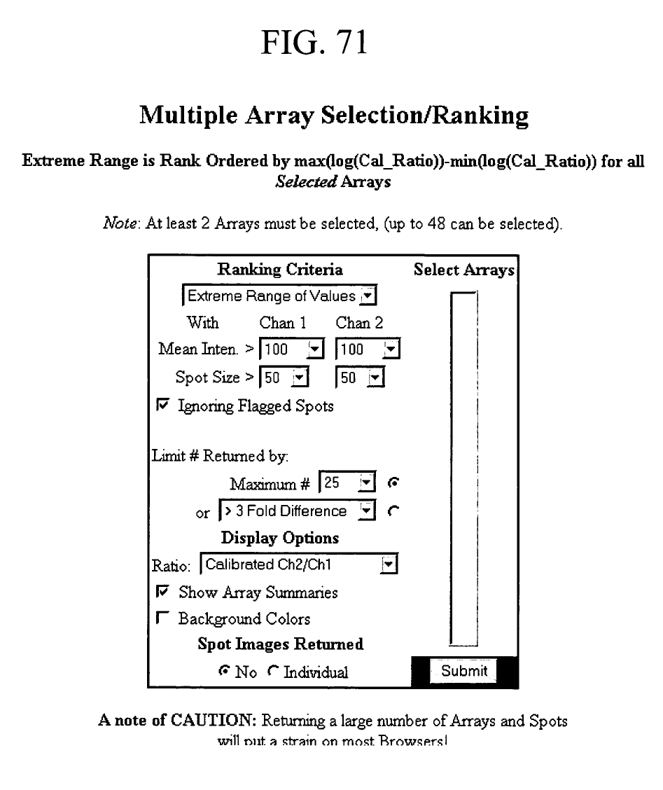

The Multi Rank/Multi Display query form shown in FIG. 71 captures three types of information.
Ranking Criteria
Experiments to be ranked
Display options

To begin, review and select the Ranking tool options:

1. Ranking Criteria can be chosen from the drop-down list. The choices are Extreme Range of Values or Maximum of Values. Extreme Range of Values uses the formula shown in the figure above [max(log(Cal_Radio))-min(log(Cal_Ratio))], ranking the results by the greatest differences among the chosen arrays. Maximum of Values ranks the results by the greatest (or least) ratio value among the chosen arrays [max(log(Cal_Ratio))].

2. Mean Intensities for Channel 1 and Channel 2 can be chosen from the drop-down lists. These values indicate intensities greater than the values in the entry box, and are usually set to values between 100 and 500.

3. Spot Size can also be selected from the drop-down lists. Only spots with size greater than indicated will be used in the ranking information. The average size of a spot is approximately 130 pixels using the ArraySuite (Yidong) software, and the minimum spot size is therefore usually set to 10-50 pixels.

4. Flagged Spots can be either included or excluded from the ranking. Checking this box will remove Flagged Spots from the ranking.

5. Limit # Returned by can be used to designate the number of rankings returned in the drop-down list. In addition, dramatically different expression patterns can also be returned even if they fall below the filtering criteria designated by intensity or spot size.

6. Multiple array experiments can be individually selected from the list box of Select Arrays. Holding down the Ctrl (for PC) or Shift key (for Mac) while selecting each array experiment allows multiple selections to be made. At least two arrays must be selected.

7. Click the Submit button to initiate the query.

Display options can be used to tailor your query outputs. The following list explains each option.

Ratio: The source of each ratio can be designated from the drop-down list provided.

Show Array Summaries checkbox can be used to display additional experimental summary information. See Results Display for an example of an Array Summary.

Background Colors checkbox can be used to display a false color scale designation for each ratio in the query results.

Spot Image Returned radio buttons can be used to choose the type of spot displayed in the results table.
No: No image will be returned if this radio button is marked.
Individual: If by chance suspect artifacts need to be confirmed, individual spots which are cut out to show 50% of the neighboring spots will be returned. This will provide a better image of the surrounding area.
   Results Display

The Array Summaries table shown in FIG. 68 provides a quick look up of summary information about an entire experiment. This table shows information about:
Array designation: Information in this column is linked to an image of each experiment. If selected, a new browser page is launched and the experimental image data are returned. This image can be resized for viewing and capturing.
Probe 1: Shows the naming convention entered designated for probe 1.
Probe 2: Shows the naming convention entered designated for probe 2.
Average Sample Intensities for Channel 1 and 2: Shows the average mean intensity based upon values set on the Multiple Array Query form.
Average Spot Sizes for Channel 1 and 2: Shows the average mean intensity based upon values set on the Multiple Array Query form.
% No Targets for Channel 1 and 2: This value represents the percentage of spots not detected by the array software program. This value provides an estimate of quality for the experiment. A good experiment might have a normal range of 1-10%. If this value is high, it may indicate that the signal intensities for one channel or the other is low/weak or that a large area of the whole slide may have a problem. Visually inspecting the array image would be recommended to determine the meaning of these values.
Calibration Factor: This is a numerical value used to adjust the ratio of the experiment so that the median ratio is equal to one. In the normal distribution of ratios within an experiment, the median will not precisely equal one because of experimental error. This is used in all the tools, and factors greater than 4-5 are unacceptable. More often, the calibration factor ranges between 0.5-2.
   Note: You can disable the Array Summaries report from the Multiple Array Query form.

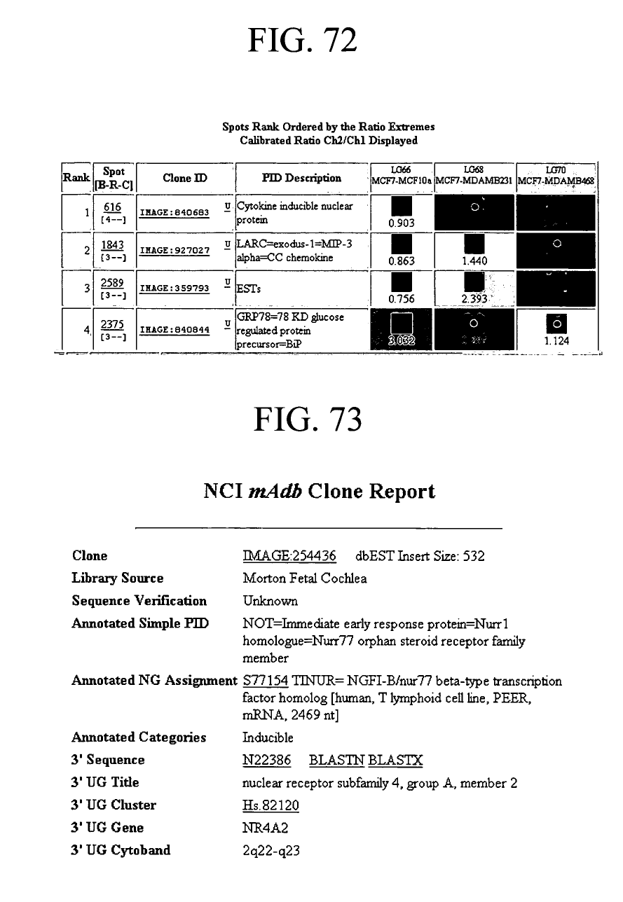

The Rank Order Query Results table shown in FIG. 72 ranks across experiments based on the ratio information showing the greatest change in ratio across the experiments

This ranking results table shows information about:
Rank: Information in this column is linked to an image of each experiment. If selected, a new browser page is launched and the experimental image data are returned. This image will be able to be resized for viewing and capturing in a future implementation.
Spot [B-R-C]: The spot link will launch a new web browser page and display the spot images for the selected experiments that correspond to the Clone ID. For example, clicking on spot number 1843 from this column would return an image such as that shown in FIG. 70
B-R-C stands for the Block-Row-Column location of the spot on the slide.
Clone ID: The Clone ID link will launch a new web browser page and display a Clone Report (see Appendix B). This report has specific clone information that is updated on a regular basis and is linked to a number of peripheral resources such as UniGene and GenBank. In addition, a direct link to the UniGene cluster information is provided (u), although this information is available in each clone report. For private clones, the designation TBA (to be assigned) is used to indicate an incomplete clone report.
PID Description: This description is a simple annotation of the clone information and represents a putative ID of the clone. Typically, gene names or title information is provided. This information is currently captured from a variety of sources (see Appendix B).
Selected Experiments: Each of the queried experiments will be designated as a single column. The experiments are returned in the ranked order as compared with the designated multiple experiments from the query form. Options for viewing the spot image and background color can be selected from the query form. Ratio information is displayed below the spot image.
   Note: The spot image will be displayed only if the Individual Spot Image Returned radio button is selected.
      Appendix A—Clone Reports

Clone Report is shown in FIG. 73. This report has specific clone information that is updated on a regular basis and is linked to a number of peripheral resources such as UniGene and GenBank. In addition, a direct link to the UniGene cluster information is provided, although this information is available in each clone report. The UniGene cluster information is automatically updated weekly to represent the most current information from the UniGene clustering results.

Definitions

Clone—The IMAGE consortium clone used to generate the target spot; hyperlinked to the dbEST record(s) with the IMAGE ID number.
Library Source—library from which the IMAGE clone was derived, taken from the dbEST record.
Sequence Verification—who confirmed the sequence from the IMAGE clone (Stanford, NCI, Unknown).
Annotated Simple PID—short Putative or Probable IDentification of the clone's homology (local annotation).
Annotated NG Assignment—Named Gene assignment which is hyperlinked to the GenBank nucleotide record via the accession number for the Named Gene.
Annotated Categories—Classification of functional role(s) of the Named Gene in the cell.
3′ Sequence—hyperlink to the GenBank record for the 3′ sequence from the IMAGE clone, as well as hyperlinks to the BLASTN and BLASTX output using the 3′ sequence as input.
3′ UG Title—title of the gene (if known) matching the 3′ sequence in the UniGene cluster database.
3′ UG Cluster—link to the UniGene database for the UniGene cluster matching the 3′ sequence.
3′ UG Gene—NCBI LocusLink name for the gene with best homology to the matching UniGene cluster sequence, with links to that gene in the GeneCards database and via Med Miner to the literature on that gene, if available.
3′ UG Cytoband—cytogenetic position of the matching UniGene cluster derived from the UniGene record.
   Appendix B—Data Capture Shortcuts
   PC shortcuts

[Alt]-[Print Screen] to print a snap shot of a window, place cursor in the window and hold down the [Alt] key and press the [Print Screen] key.

[Ctrl]-[v] to paste the PC window shot into another document, hold down the [Ctrl] key and press the letter [v].

Appendix C—The following references are hereby incorporated by reference herein:

1. Ermolaeva, O., Rastogi, M., Pruitt, K. D., Schuler, G. D., Bittner, M. L., Chen, Y., Simon, R., Meltzer, P., Trent, J. M., and Boguski, M. S. (1998) Nat Genet, 20(1), 19-23.
2. Chen, Y., Dougherty, E. R., and Bittner, M. L. (1997) Journal of Biomedical Optics, 2(4), 364-374.
3. Eisen, M. B., Spellman, P. T., Brown, P. O., and Botstein, D. (1998) Proc Natl Acad Sci USA, 95(25), 14863-8.

### EXAMPLE 35

### Exemplary Definitions

When used in any of the examples described herein, the following terms can be defined as described below.

Gene expression is conversion of genetic information encoded in a gene into RNA and protein, by transcription of a gene into RNA and (in the case of protein-encoding genes) the subsequent translation of mRNA to produce a protein. Hence, expression involves one or both of transcription or translation. Gene expression is often measured by quantitating the presence of mRNA.

Gene expression level is any indication of gene expression, such as the level of mRNA transcript observed in biological material. A gene expression level can be indicated comparatively (e.g., up by an amount or down by an amount) and, further, may be indicated by a set of discrete values (e.g., up-regulated, unchanged, or down-regulated).

A probe comprises an isolated nucleic acid which, for example, may be attached to a detectable label or reporter molecule, or which may hybridize with a labeled molecule. For purposes of the present disclosure, the term “probe” includes labeled RNA from a tissue sample, which specifically hybridizes with DNA molecules on a cDNA microarray. However, some of the literature describes microarrays in a different way, instead calling the DNA molecules on the array “probes.” Typical labels include radioactive isotopes, ligands, chemiluminescent agents, and enzymes. Methods for labeling and guidance in the choice of labels appropriate for various purposes are discussed, e.g., in Sambrook et al., Molecular Cloning: A Laboratory Manual, Cold Spring (1989) and Ausubel et al., Current Protocols in Molecular Biology, Greene Publishing Associates and Wiley-Intersciences (1987).

Hybridization: Oligonucleotides hybridize by hydrogen bonding, which includes Watson-Crick, Hoogsteen or reversed Hoogsteen hydrogen bonding between complementary nucleotide units. For example, adenine and thymine are complementary nucleobases which pair through formation of hydrogen bonds. “Complementary” refers to sequence complementarity between two nucleotide units. For example, if a nucleotide unit at a certain position of an oligonucleotide is capable of hydrogen bonding with a nucleotide unit at the same position of a DNA or RNA molecule, then the oligonucleotides are complementary to each other at that position. The oligonucleotide and the DNA or RNA are complementary to each other when a sufficient number of corresponding positions in a molecule are occupied by nucleotide units which can hydrogen bond with each other.

### EXAMPLE 36

### Exemplary Alternate Applications of Technology

As described in the examples, the technologies can be applied to a wide range of applications. In addition, the technologies can be applied to pharmacologic response studies (e.g., matching tumors with chemotherapy or persons with toxic responses to specific drugs). Other applications include research applications on animal models (e.g., mouse models of cancers or immune disease participating in studies to link gene expression with response). Still other applications include research on bacteria (e.g., used to screen response to new antibiotics).

### EXAMPLE 37

### Exemplary Alternatives

Although, for simplicity, the present document often makes reference to “genes” (e.g., as can be represented by gene expression profiles, transcriptional rate, transcript levels, etc.), the technologies described herein can be applied to the analysis of any biological response profile. In particular, the methods of the disclosed system are equally applicable to biological profiles which comprise measurements of other cellular constituents such as, but not limited to, measurements of any nucleic acid and measurements of protein abundance or protein activity levels.

Further, any test result, such as DNA sequencing, Restriction Fragment Length Polymorphism (“RFLP”) analysis, and the like, can be added to the databases. Still other data that can be added includes Single nucleotide polymorphism (“SNP”) analyses, profiling genome for polymorphisms and results from antibody arrays (used to interrogate samples for the presence of proteins or other antigens) or protein chips, including via the Surface-Enhanced Laser Desorption/Ionization “SELDI” or Matrix Assisted Laser Desorption/Ionization-Time of Flight Mass Spectrometry (“MALDI-TOF”) processes.

Although any of the examples can be directed to human subjects, the technology can alternatively be applied to other subjects (e.g., any other biological organism, including plant, animal, and bacterium subjects).

For those actions specified as computer-executable, such actions can be performed fully-automatically (e.g., without human intervention) or semi-automatically (e.g., with assistance from a human operator). One or more computer-readable media can comprise the instructions described as computer-executable.

In view of the many possible embodiments to which the principles of the invention may be applied, it should be recognized that the illustrated embodiments are examples of the invention, and should not be taken as a limitation on the scope of the invention. Rather, the scope of the invention includes what is covered by the following claims. We therefore claim as our invention all that comes within the scope and spirit of these claims.

1. A computer-implemented method comprising:
receiving a query specifying one or more non-gene criteria for subjects for which non-gene data and gene expression data is stored; and
providing data indicating gene expression data for a subset of the subjects meeting the non-gene criteria.

2. The method of claim 1 wherein the non-gene criteria comprise epidemiological criteria for the subjects.

3. The method of claim 2 wherein the non-gene criteria further comprise demographic criteria for the subjects.

4. The method of claim 2 wherein the non-gene criteria comprise disease status for the subjects.

5. The method of claim 2 wherein the non-gene criteria comprise disease symptoms for the subjects.

6. The method of claim 2 wherein the non-gene criteria comprise clinical test results for the subjects.

7. The method of claim 2 wherein the non-gene criteria comprise body mass index for the subjects.

8. The method of claim 1 wherein the non-gene criteria comprise demographic criteria for the subjects.

9. The method of claim 8 wherein the non-gene criteria comprise age for the subjects.

10. The method of claim 1 wherein the non-gene criteria are received via an HTML form.

11. The method of claim 1 further comprising:
after displaying data indicating gene expression data for a subset of the subjects meeting the non-gene criteria, accepting additional non-gene criteria; and
performing a query on the subset with the additional non-gene criteria.

12. The method of claim 1 further comprising:
after displaying data indicating gene expression data for a subset of the subjects meeting the non-gene criteria, accepting gene expression criteria; and
performing a query on the subset with the gene expression criteria.

13. The method of claim 12 wherein the gene expression criteria comprise a threshold value for use in determining whether a gene is expressed in an individual.

14. The method of claim 12 wherein the gene expression criteria comprise a number of subjects threshold value for use in determining whether a gene is expressed within a group.

15. The method of claim 1 further comprising:
receiving a manual selection of selected one or more subjects; and
applying an analysis tool analyzing gene expression data of the selected subjects against gene expression data for other subjects.

16. The method of claim 1 further comprising:
receiving one or more other non-gene criteria, the other non-gene criteria comprising grouping criteria; and
grouping the gene expression data into a plurality of groups based on the grouping criteria.

17. The method of claim 16 further comprising:
presenting an analysis of the gene expression data for at least one of the groups vis-à-vis at least one other of the groups.

18. The method of claim 17 wherein the analysis comprises determining which genes are expressed in one group but not another.

19. The method of claim 18 further comprising:
displaying how many genes are expressed in one group but not another.

20. The method of claim 18 further comprising:
displaying the names of genes expressed in one group but not another.

21. The method of claim 20 further comprising:
responsive to a user selection of one of the names, accessing a public database entry for a gene associated with the name; and
displaying the public database entry.

22. The method of claim 17 wherein the analysis comprises determining which genes are expressed in both of two groups.

23. The method of claim 17 wherein the analysis comprises a visual depiction of hierarchical clustering.

24. The method of claim 1 further comprising:
presenting a list of microarray experiments associated with the subjects meeting the non-gene criteria;
accepting a selection of at least two of the microarray experiments as selected microarray experiments; and
depicting a visual comparison of the selected microarray experiments.

25. The method of claim 24 wherein the visual comparison comprises a scatter plot of gene expression information associated with the selected microarray experiments.

26. The method of claim 25 wherein gene expression information for one of the selected microarrays is compared to gene expression information for a plurality of other of the selected microarray experiments.

27. The method of claim 25 wherein gene expression information for one of the selected microarrays is compared to gene expression information for an other of the selected microarray experiments for a plurality of pairs of selected microarray experiments.

28. The method of claim 24 wherein the visual comparison comprises an M v. A plot associated with the selected microarray experiments.

29. The method of claim 28 further comprising:
in a graphical user interface, presenting a minimum-intensity slider by which the minimum intensity for displayed data is manipulated.

30. The method of claim 1, wherein the method is employed to profile a disease.

31. The method of claim 1, wherein the method is employed to discover disease biomarkers.

32. The method of claim 1, wherein the method is employed to analyze data from a clinical trial.

33. A computer-readable medium comprising computer-readable instructions for performing the method of claim 1.

34. A data processing system comprising:
a gene expression data store comprising one or more gene expression fields for a plurality of subjects;
a non-gene data store comprising one or more non-gene fields for the plurality of subjects;
wherein the data processing system comprises at least one data structure in one or more computer-readable storage media for linking the gene expression data store and the non-gene data store whereby a query comprising non-gene criteria is operable to return associated data from the gene expression data store.

35. The data processing system of claim 34 wherein the data structure comprises a database field.

36. The data processing system of claim 34 wherein the data structure comprises a database table.

37. The data processing system of claim 34 further comprising a query engine operable to process the query.

38. The data processing system of claim 34 wherein the query comprises an epidemiological criterion.

39. The data processing system of claim 38 wherein the query comprises a body mass index for the subjects.

40. The data processing system of claim 34 wherein the query comprises a demographic criterion.

41. The data processing system of claim 34 further comprising an HTML user interface generator for acquiring values specified for the non-gene criteria.

42. The data processing system of claim 34 further comprising a data structure into which microarray experiment data can be uploaded via specifying an file name.

43. The data processing system of claim 34 wherein
the criteria comprise epidemiological and demographic criteria; and
the query operates to retrieve gene expression profiles for subjects meeting the criteria.

44. The data processing system of claim 43 wherein the query further operates to group subjects into two or more groups based on grouping criteria.

45. The data processing system of claim 44 wherein the grouping criteria comprise whether a subject is a control subject.

46. The data processing system of claim 44 wherein the grouping criteria comprise at least one selected from the group consisting of the following:
age;
gender;
body mass index;
race; and
disease status information.

47. The data processing system of claim 34, further comprising tools for performing at least one selected from the group consisting of the following:
preprocessing;
visualization;
group comparisons;
discriminant analysis;
group discovery; and
cluster analysis.

48. The data processing system of claim 34 wherein the tools comprise one or more selected from the group consisting of the following:
normalization;
estimation of missing values;
subsetting based on percent of missing data or significance of gene expression difference;
gene expression distributions;
quantile-quantile plots;
scatter plots;
visual comparisons via scatter plots;
principal component analysis;
multi-dimensional scaling;
visual exploratory analysis of correlation matrix;
discriminate analysis;
significance tests;
validation via permutation tests;
hierarchical clustering;
Kmeans clustering; and
SOM clustering.

49. The data processing system of 34 further comprising a data structure comprising a link to a public external database.

50. The data processing system of claim 34 wherein the gene expression data comprises gene expression level observations generated by subjecting sample biological material to an experimental condition and observing regulation of mRNA transcription levels for a plurality of genes in the biological material as a result of being subjected to the experiment.

51-82. (canceled)

83. One or more computer-readable storage media comprising computer-executable instructions for performing a method comprising:
receiving a query specifying one or more non-gene criteria for subjects for which non-gene data and gene expression data is stored; and
providing data indicating gene expression data for a subset of the subjects meeting the non-gene criteria.
